SEQUENCE LISTING (18 sequences)

SEQ ID NO: 1 - protein, 6 aa, Synthetic construct
VVVGEE

SEQ ID NO: 2 - protein, 6 aa, Synthetic construct
VVVGKK

SEQ ID NO: 3 - skipped

SEQ ID NO: 4 - protein, 20 aa, Synthetic construct
VVVGKKGGOEGOEGOEGOEG

SEQ ID NO: 5 - protein, 26 aa, Synthetic construct
GDYSHCSPLRYYPWWKCTYPDPEGGG

SEQ ID NO: 6 - protein, 46 aa, Synthetic construct
VVVGKKGGOEGOEGOEGOEGGDYSHCSPLRYYPWWKCTYPDPEGGG

SEQ ID NO: 7 - protein, 46 aa, Synthetic construct
VVVGKKGGOEGOEGOEGOEGYPKYPCTGSGSDGDELCGWYPHWPYR

SEQ ID NO: 8 - protein, 26 aa, Synthetic construct
YPKYPCTGSGSDGDELCGWYPHWPYR

SEQ ID NO: 9 - protein, 21 aa, Synthetic construct
GSRIGCRDSRCNWWAPGEGGG

SEQ ID NO: 10 - protein, 21 aa, Synthetic construct
GSRGFCRDSSCSFPAPGEGGG

SEQ ID NO: 11 - protein, 22 aa, Synthetic construct
AGWEVCHWAPMMCKHGGTEGGG

SEQ ID NO: 12 - protein, 22 aa, Synthetic construct
AGSDWCGTWNNPCFHQGTEGGG

SEQ ID NO: 13 - protein, 26 aa, Synthetic construct
GDRLHCKPQRQSPWMKCQHLDPEGGG

SEQ ID NO: 14 - protein, 26 aa, Synthetic construct
GDLHACRPVRGDPWWACTLGDPEGGG

SEQ ID NO: 15 - protein, 26 aa, Synthetic construct
GDRYLCLPQRDKPWKFCNWFDPEGGG

SEQ ID NO: 16 - protein, 26 aa, Synthetic construct
GDYSHCSPLRYYPWWKCTYPDPEGGG

SEQ ID NO: 17 - protein, 26 aa, Synthetic construct
GDGFTCSPIRMFPWFRCDLGDPEGGG

SEQ ID NO: 18 - protein, 26 aa, Synthetic construct
GDFSPCKALRHSPWWVCPSGDPEGGG

(19) United States
(12) Patent Application Publication
(10) Pub. No.: US 2025/0009896 A1
(43) Pub. Date: Jan. 9, 2025

(54) SUPRAMOLECULAR FILAMENTS BASED VIRAL PARTICLE CAPTURE FOR PREVENTION AND THERAPY OF VIRAL INFECTION

(71) Applicant: The Johns Hopkins University, Baltimore, MD (US)

(72) Inventors: Honggang CUI, Lutherville, MD (US); Hongpeng Jia, Pikesville, MD (US); Caleb French Anderson, Baltimore, MD (US)

(21) Appl. No.: 18/587,056

(22) Filed: Feb. 26, 2024

Related U.S. Application Data
(60) Provisional application No. 63/486,881, filed on Feb. 24, 2023.

(51) Int. Cl. A61K 47/64; A61K 9/00; A61K 38/00; A61P 31/14

(52) U.S. Cl. CPC A61K 47/64; A61K 9/0043; A61K 9/0078; A61K 38/005; A61P 31/14

(57) ABSTRACT

A supramolecular filament comprising an ACE2-binding ligand peptide amphiphile, a self-assembling filler peptide amphiphile, and a soluble ACE; and related compositions, respirable aerosols or nasal sprays, and methods for treating or preventing a viral infection are disclosed.

### CROSS-REFERENCE TO RELATED APPLICATIONS

This application claims benefit of U.S. Provisional Patent Application No. 63/486,881 filed Feb. 24, 2023, which is incorporated herein by reference in its entirety.

### FEDERALLY SPONSORED RESEARCH OR DEVELOPMENT

This invention was made with government support under grants 1R21AI14932101 and 3R21AI149321-01S1 awarded by the National Institutes of Health. The government has certain rights in the invention.

### SEQUENCE LISTING

The instant application contains a Sequence Listing which has been submitted electronically in XML file format and is hereby incorporated by reference in its entirety. The XML copy, created on Feb. 26, 2024, is named “JHU_41721_202_SequenceListing.xml” and is 16,259 bytes in size.

### TECHNICAL FIELD

The present disclosures relates to a supramolecular filament and related compositions, pharmaceutical formulations, respirable aerosol or nasal sprays, kits, methods for treating or preventing a viral infection, methods for silencing ACE2 enzymatic activity, and methods for inhibiting viral entry in a subject.

### BACKGROUND

Coronaviruses have recently precipitated a global pandemic of severe acute respiratory syndrome (SARS) into devastating public health crises. Despite the virus's rapid rate of mutation, all SARS-CoV-2 variants are known to gain entry into the host cells primarily through complexation with angiotensin converting enzyme-2 (ACE2). Although ACE2 has potential as a druggable decoy to block viral entry, its clinical use is complicated by its essential biological role as a carboxypeptidase and further hindered by its structural and chemical instability.

### SUMMARY

In some aspects, the presently disclosed subject matter provides a supramolecular filament comprising: an ACE2-binding ligand peptide amphiphile (PA) comprising, in order from N-terminus to C-terminus: i) an aliphatic segment, ii) an intermolecular interaction-regulating peptide segment, iii) a hydrophilic spacer, and iv) an ACE2 inhibitor, wherein the intermolecular interaction-regulating peptide segment of the ACE2-binding ligand peptide comprises, in order from N-terminus to C-terminus: a hydrogen bond-contributing sequence and one or more charged amino acid residues; a self-assembling filler peptide amphiphile (PA) comprising, in order from N-terminus to C-terminus: i) an aliphatic segment, and ii) an intermolecular interaction-regulating peptide segment, wherein the intermolecular interaction-regulating peptide segment of the self-assembling filler PA comprises, in order from N-terminus to C-terminus: a hydrogen bond-contributing sequence and one or more charged amino acid residues that have an opposite charge to the charged amino acid residues of the ACE2-binding ligand PA; and a soluble ACE2 comprising an S-protein binding site and a carboxypeptidase active site, wherein the soluble ACE2 is bound at the carboxypeptidase active site to the ACE2 inhibitor.

In some aspects, the aliphatic segment of the ACE2-binding ligand PA or the self-assembling filler PA comprises a dodecyl chain. In some aspects, the charged amino acid residues of the self-assembling filler PA comprises: (i) one or more negatively charged glutamic acid residues; or (ii) one or more positively charged lysine residues. In some aspects, the charged amino acid residues of the ACE2-binding ligand PA comprises one or more positively charged lysine residues and the charged amino acid residues of the self-assembling filler PA comprises one or more negatively charged glutamic acid residues. In some aspects, the self-assembling filler PA and the ACE2-binding ligand PA form a filamentous structure comprising electrostatic complexation between the charged amino acid residues of the ACE2-binding ligand PA and the charged amino acid residues of the self-assembling filler PA.

In some aspects, the hydrogen bond-contributing sequence of the self-assembly filler PA comprises a sequence of VVV (SEQ ID NO: 3). In some aspects, the hydrogen bond-contributing sequence of the ACE2-binding ligand PA comprises a sequence of VVV (SEQ ID NO. 3). In some aspects, the self-assembling filler PA and the ACE2-binding ligand PA form a filamentous structure comprising a hydrogen bonding network.

In some aspects, the intermolecular interaction-regulating peptide segment of the ACE2-binding ligand PA comprises a sequence of VVVGKK (SEQ ID NO: 2) and the intermolecular interaction-regulating peptide segment of the self-assembling filler PA comprises a sequence of VVVGEE (SEQ ID NO: 1).

In some aspects, the hydrophilic spacer comprises a hydrophilic oligoethylene glycol (OEG) chain having between about 4 to 16 OEG units. In some aspects, the OEG chain has a length selected from 4, 8, 10, 12, 14, and 16 units. In some aspects, the OEG chain comprises 4 units. In some aspects, the hydrophilic spacer further comprises a double glycine (GG) sequence.

In some aspects, the ACE2 inhibitor comprises a chemical structure of:

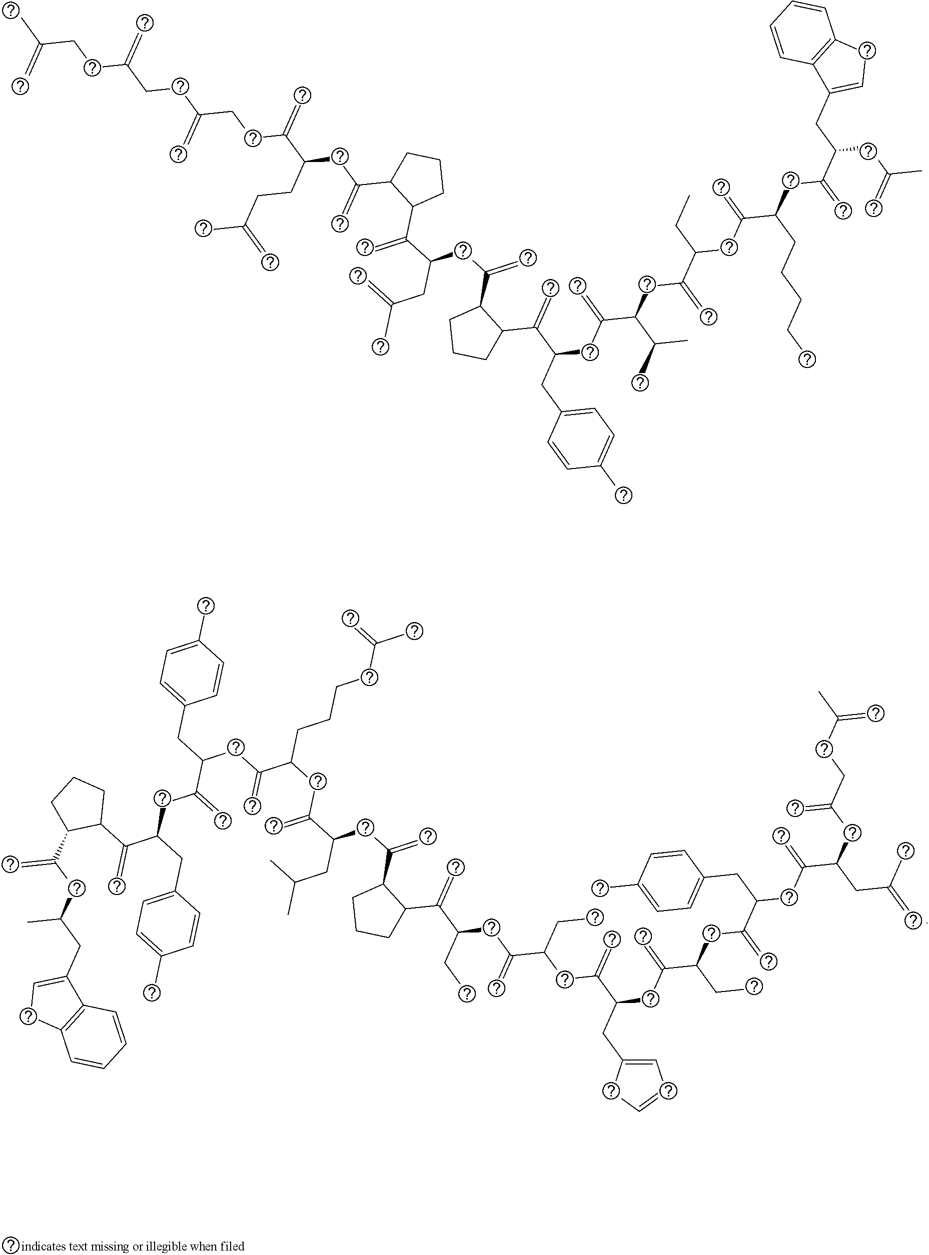

In some aspects, the ACE2-binding ligand PA comprises amino acid sequence GDYSHCSPLRYYPWWKCTYPDPEGGG (SEQ ID NO:5). In some aspects, the ACE2-binding ligand PA comprises amino acid VVVGKKGGOEGOEGOEGOEGGDYSHCSPLRYYPWWKCTYPDPEGGG (SEQ ID NO: 6). In some aspects, the ACE2-binding ligand PA comprises the following structure:

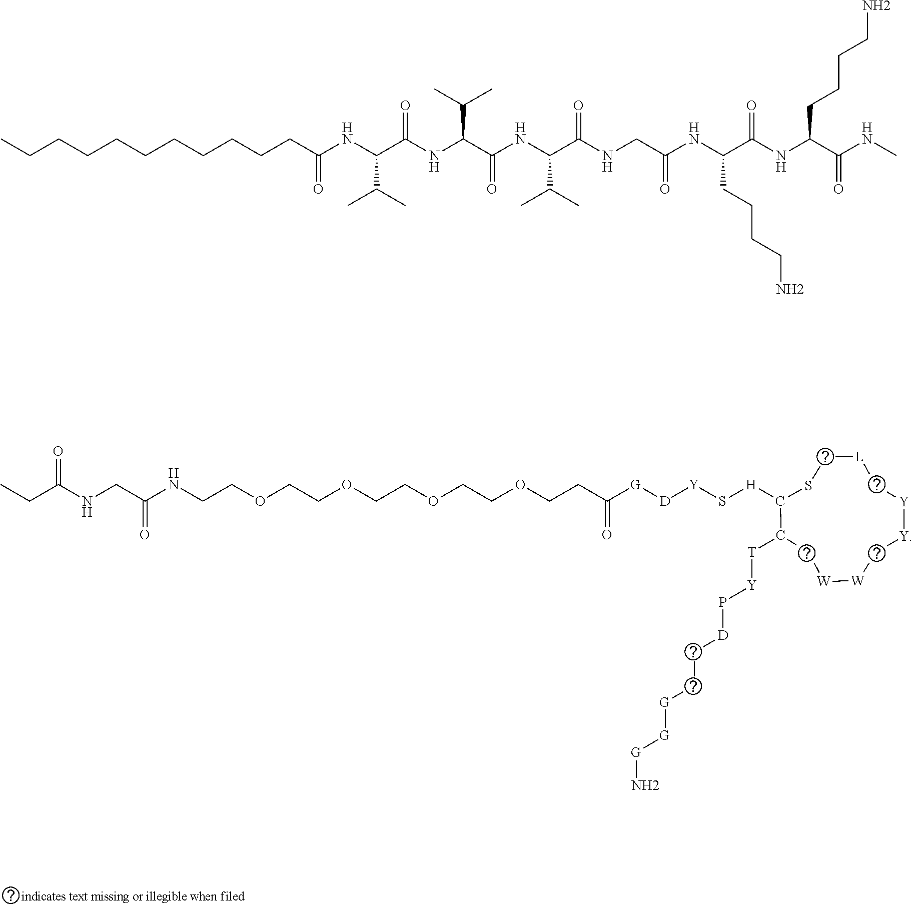

In some aspects, the ACE2-binding ligand PA has the following chemical structure:

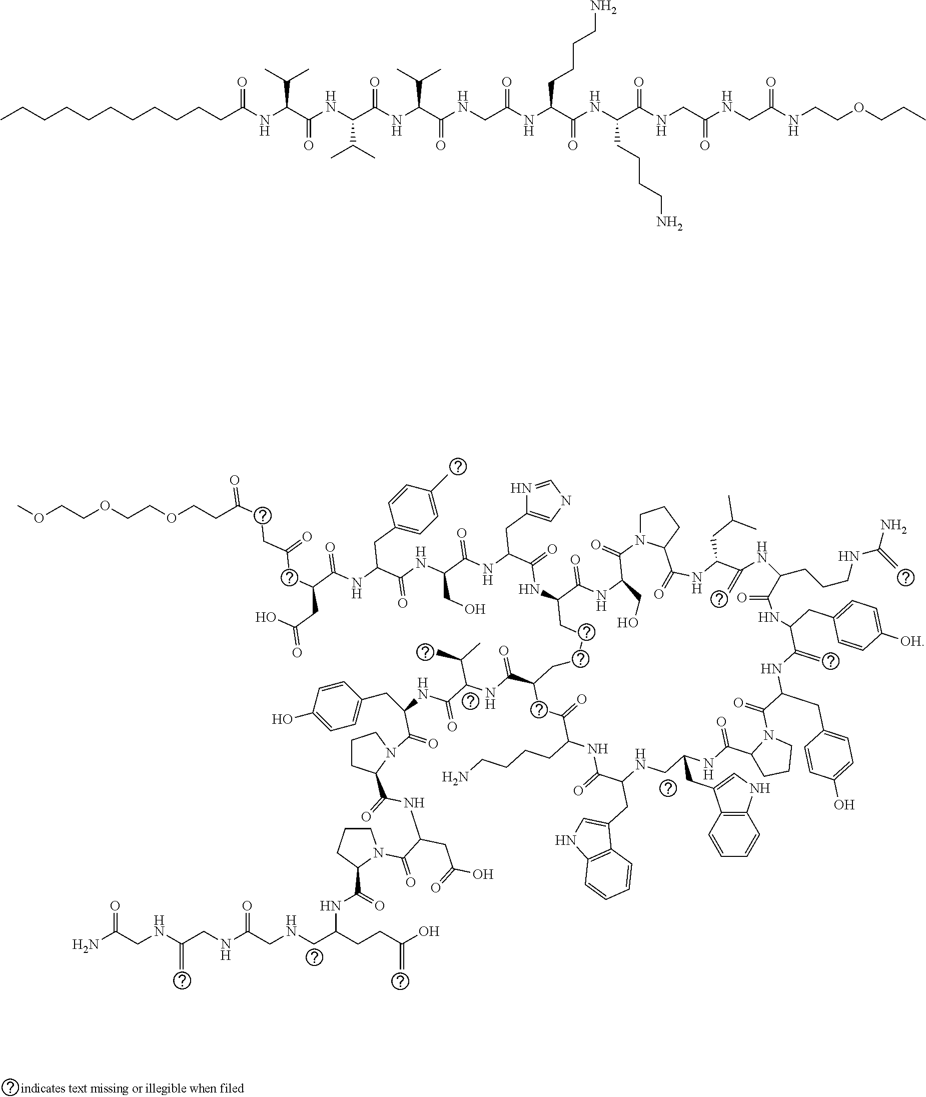

In some aspects, the intermolecular interaction-regulating peptide segment of the self-assembling filler PA comprises a sequence of VVVGEE (SEQ ID NO: 1). In some aspects, the self-assembling filler PA comprises the following chemical structure:

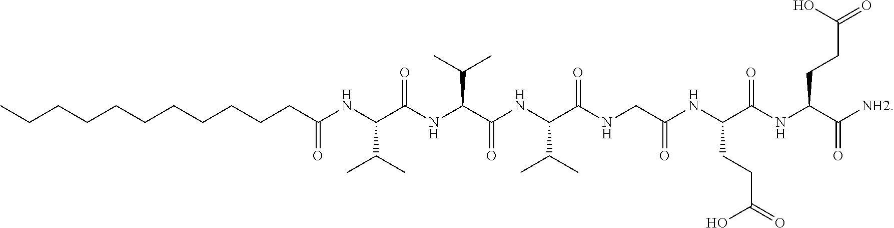 [SMILES: CCCCCCCCCCCC(=O)N[C@H](C(=O)N[C@H](C(=O)N[C@H](C(=O)NCC(=O)N[C@@H](CCC(=O)O)C(=O)N[C@@H](CCC(=O)O)C(N)=O)C(C)C)C(C)C)C(C)C]

In some aspects, the supramolecular filament comprises a molar ratio of the self-assembling filler PA to the ACE2-binding ligand PA in the range of about 10:1 to about 50:1. In some aspects, the molar ratio of filler to ligand is about 20:1.

In some aspects, the supramolecular filament is biodegradable. In some aspects, the supramolecular filament has a shape adapted to enhance deposition and retention of the filament within a lung of a subject.

In some aspects, the presently disclosed subject matter provides a composition comprising the supramolecular filament described herein. In other aspects, the presently disclosed subject matter provides a pharmaceutical formulation comprising the presently disclosed supramolecular filament and a pharmaceutically acceptable carrier. In other aspects, the presently disclosed subject matter provides a kit comprising the presently disclosed supramolecular filament, pharmaceutical formulation, and/or respirable aerosol or nasal spray and instructions for use.

In some aspects, the presently disclosed subject matter provides a respirable aerosol or nasal spray comprising the supramolecular filament described herein.

In some aspects, the presently disclosed subject matter provides a method for treating or preventing a viral infection, the method comprising administering to a subject an amount of the supramolecular filament described herein effective to inhibit viral entry into host cells. In some aspects, the method treats or prevents a lung injury or respiratory illness. In particular aspects, the respiratory illness comprises coronavirus disease of 2019 (COVID-19). In some aspects, the viral infection comprises a coronavirus infection. In some aspects, the coronavirus infection comprises a severe acute respiratory syndrome coronavirus (SARS-CoV) or SARS-CoV-2 infection. In some aspects, the coronavirus comprises coronavirus disease of 2019 (COVID-19). In some aspects, the supramolecular filament is administered in a respirable aerosol or a nasal spray.

In some aspects, the supramolecular filament described herein is delivered directly to the subject's nasal cavity or lungs. In some aspects, the supramolecular filament is deposited within a mucus layer (through charge interactions) of the subject's nasal cavity or lungs. In some aspects, the supramolecular filament described herein is presented over a lining of an epithelia of the subject's nasal cavity or lungs. In some aspects, the supramolecular filament binds to the coronavirus. In some aspects, the coronavirus bound to the supramolecular filament is cleared or degraded by one or more clearance mechanisms. In some aspects, the one or more clearance mechanisms is associated with a macrophage or a mucociliary escalator.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying drawings, which are not necessarily drawn to scale, and wherein:

FIG. 1A, FIG. 1B, FIG. 1C, FIG. 1D, FIG. 1E, FIG. 1F, FIG. 1G, FIG. 1H, FIG. 1I, and FIG. 1J show the design of the presently disclosed ACE2-docking and silencing supramolecular filaments and their assembly in aqueous solution. (FIG. 1A) Cartoon representation of soluble ACE2 alongside its protein structure with the SARS-CoV-2 S-protein RBD-binding site on ACE2 highlighted in yellow (RCSB Protein Data Bank 6M17) and the cleft of the carboxypeptidase active site of ACE2 highlighted in cyan (RCSB Protein Data Bank 1R42), where protein structure was generated using UCSF ChimeraX software. (FIG. 1B) Chemical structure of ACE2-binding Ligand (top) and Filler (bottom) PA molecules investigated, where both contain the same aliphatic region (C₁₂) in black and the same intermolecular hydrogen bond-contributing sequence (VVV) in blue. The Filler PA contains negatively charged glutamic acid residues (EE) (cyan) to pair with the positively charged lysine residues (KK) (red) of the Ligand PA. The flexible, hydrophilic spacer (OEG₄) (green) of the Ligand PA distances the ACE2-binding peptide, DX600 (pink), from the surface of assembled supramolecular structures. (FIG. 1C) The two PAs can be dissolved together in aqueous solutions to spontaneously associate and co-assemble into supramolecular filaments that display the ACE2-binding ligand on their surfaces. Subsequently, soluble ACE2 can be added to filament solutions to allow for binding via inhibition of the ACE2 proteolytic active site to yield decoy ACE2-docking filaments, called fACE2. (FIG. 1D) fACE2 can be delivered to nasal passageways and lung tissues where it can bind to and capture SARS coronaviruses to block viral entry into host cells. (FIG. 1E) Representative low magnification transmission electron microscopy (TEM) images of ACE2-docking supramolecular filaments (20:1 molar ratio of Filler to Ligand) after dissolving at 1 mM in PBS at pH=7.4 and aging for 24 h, revealing ribbon-like filaments over several microns in length. (FIG. 1F) High magnification TEM image of boxed area marked in (FIG. 1E). Filament diameter represented as mean±SD (n=35). (FIG. 1G-FIG. 1J) Representative cryogenic-TEM micrographs of ACE2-docking supramolecular filaments formed from co-assembly of varying molar ratios of Filler to Ligand (fixed 50 μM Ligand concentration: (FIG. 1G) 1:0, (FIG. 1H) 10:1, (FIG. 1I) 20:1, and (FIG. 1J) 50:1) after dissolving in PBS at pH=7.4 and aging for 24 h, confirming ribbon-like morphology with slight twisting;

FIG. 2A, FIG. 2B, FIG. 2C, FIG. 2D, FIG. 2E, FIG. 2F, FIG. 2G, FIG. 2H, and FIG. 2I show docking ACE2 to supramolecular filament surfaces via enzyme-substrate complexation. Biolayer interferometry-based analysis of the interaction kinetics of Ligand and sLigand with immobilized ACE2 by 3-fold dilution (33.3 to 0.137 μM). (FIG. 2A) Equilibrium response signal of Ligand and sLigand evaluated at moment before dissociation step (299 s). Response signals are normalized to their respective maximum values. BLI kinetic traces of (FIG. 2B) Ligand and (FIG. 2C) sLigand association to immobilized ACE2 with dissociation step occurring at 300 s. (FIG. 2D) Kinetic measurement of evolved fluorescence of activity probe by ACE2 cleavage in the presence of various ACE2-docking filament components (ACE2=free rhACE2; 20:1 Fil=20:1 molar ratio of Filler to Ligand). Data presented as mean±SD (n=3). (FIG. 2E) Initial velocity calculated from kinetic measurements of ACE2 activity in the presence of ACE2-docking filament components. Data presented as mean±SD (*p<0.05; **p<0.01; ns p>0.05; ****p<0.0001 for ACE2, Filler, sLigand vs. every other group; one-way ANOVA with Tukey's post hoc test, n=3). (FIG. 2F) Zeta potential measurements of Ligand and sLigand before and after incubation with ACE2. Data presented as mean±SD (***p<0.001, nsp>0.05 otherwise, one-way ANOVA with Tukey's post hoc test, n=3). Optimization of co-assembly and loading parameters to maximize ACE2 docking to supramolecular filaments, determined by extent of enzymatic inhibition relative to free ACE2 control. (FIG. 2G) Impact of Filler:Ligand molar ratio on docking efficiency, holding Ligand (50 μM) and ACE2 (50 nM) concentration fixed while varying Filler concentration. Data presented as mean±SD (**p<0.01, ns p>0.05, ****p<0.0001 otherwise, one-way ANOVA with Tukey's post hoc test, n=3). (FIG. 2H) Impact of Ligand:ACE2 molar ratio on docking efficiency, holding Ligand (50 μM) concentration and Filler:Ligand molar ratio (20:1) fixed while varying Ligand:enzyme ratio by adjusting ACE2 concentration. Data represent mean±SD (*p<0.05, ns p>0.05, ****p<0.0001 otherwise, one-way ANOVA with Tukey's post hoc test, n=3). (FIG. 2I) Impact of Ligand concentration on docking efficiency, holding Filler (1 mM) concentration and Ligand:ACE2 molar ratio (1000:1) fixed while varying Ligand concentration. Data presented as mean±SD (ns p>0.05, ****p<0.0001 otherwise, one-way ANOVA with Tukey's post hoc test, n=3);

FIG. 3A, FIG. 3B, FIG. 3C, FIG. 3D, and FIG. 3E demonstrate delivery of fACE2 within respirable aerosols via jet nebulization. (FIG. 3A) Kinetic measurement of evolved fluorescence intensity of activity probe by ACE2 cleavage post-nebulization, highlighting increased ACE2 activity afforded by fACE2 compared to free ACE2. Data represent mean SD (n=3). (FIG. 3B) Estimated initial velocities of ACE2 activity determined from kinetic measurements (relative to free ACE2 control), emphasizing preservation effect afforded by docking strategy. Data presented as mean±SD (**p<0.01, ***p<0.001, ****p<0.0001 for ACE2 vs. NEB ACE2, one-way ANOVA with Tukey's post hoc test, n=3). (FIG. 3C) Schematic illustration of hypothesized mechanism of ACE2 structural preservation afforded by fACE2, where binding affinity interactions of ACE2 at filament surface and enrichment of air-liquid interface (ALI) by filament PA monomers mitigate interaction strength of ALI on ACE2 together prevent protein unfolding and aggregation thereby preserving ACE2 activity. (FIG. 3D) Representative transmission electron micrographs of fACE2 at 1 mM in PBS at pH=7.4 before (left) and after (right) jet nebulization, showing retention of filament shape but reduction in length. Diameter measurements represented as mean±SD (n=35). (FIG. 3E) Population size distribution of observed filament contour lengths after jet nebulization of fACE2 (20 bins, 50 nm each) from TEM images, where average contour length ( ) is given as mean±SD alongside median (M) length (n=398 analyzed filaments);

FIG. 4A, FIG. 4B, FIG. 4C, FIG. 4D, FIG. 4E, FIG. 4F, and FIG. 4G demonstrate that fACE2 inhibits pseudotyped virus infection and docking of ACE2 extends its decoy function. (FIG. 4A) Schematic illustration of study design for assessing the decoy effect of fACE2. Inhibitory activity of fACE2 and controls (ACE2 and empty filaments, fil) against PsV (FIG. 4B) SARS-CoV-2, (FIG. 4C) SARS-CoV, and (FIG. 4D) VSV viral entry. Data presented as mean±SD (for highest tested dose: ns p>0.05, ***p<0.001, ****p<0.0001 for fACE2 and ACE2 vs. fil for SARS-CoV-2 and SARS-CoV PsV; one-way ANOVA with Tukey's post-hoc test, n=3 independent experiments). (FIG. 4E) Schematic illustration of study design for assessing the preventative effect of fACE2 against SARS-CoV-2 PsV challenge. (FIG. 4F) Inhibitory activity of fACE2 and controls after set incubation times against SARS-CoV-2 PsV viral entry evaluated at 0.5 nM ACE2 or equivalent dose. Data presented as mean±SD (ns p>0.05, *p<0.05, **p<0.01, ***p<0.001, with fACE2 vs. ACE2 represented above fACE2 line and ACE2 vs. fil above ACE2 line, ****p<0.0001 for fACE2 and ACE2 vs. fil otherwise; one-way ANOVA with Tukey's post-hoc test, n=3 independent experiments). (FIG. 4G) Inhibitory activity of fACE2 and fil after set incubation times (continuing from panel (FIG. 4F) against SARS-CoV-2 PsV viral entry. Data presented as mean±SD (for each time point: ns p>0.05, *p<0.05, ***p<0.001, unpaired two-tailed t-test with Welch's correction, n=3 independent experiments);

FIG. 5A, FIG. 5B, FIG. 5C, FIG. 5D, FIG. 5E, FIG. 5F, FIG. 5G, and FIG. 5H demonstrate that fACE2 attenuates viral load and lung injury after SARS-CoV-2 inoculation in vivo. (FIG. 5A) Experimental timeline to assess effectivity of fACE2 and controls delivered via inhalation in mitigating viral infectivity of subsequent SARS-CoV-2 inoculation (1.5×10⁵ TCID₅₀ dose) in K18-hACE2 transgenic mice. (FIG. 5B) Cycle thresholds of SARS-CoV-2 in harvested lungs of K18-hACE2 mice after treatment with atomized fACE2 (20 nM ACE2 dose in PBS) and controls (Ctrl, PBS; Fil, atomized equivalent empty filaments dose in PBS; rhACE2, recombinant human ACE2, 20 nM dose in PBS). Data presented as mean±SD (*p<0.05, ns p>0.05 otherwise, one-way ANOVA with Tukey's post-hoc test, n=8 mice per group). (FIG. 5C) Change in mouse body weight as a percentage of initial weight 2 days after treatment and SARS-CoV-2 inoculation. Data presented as mean±SD (nsp>0.05, one-way ANOVA with Tukey's post-hoc test, n=8). (FIG. 5D) Proinflammatory cytokine interleukin-6 (IL-6) expression from harvested mice lungs by RT-qPCR after treatment and SARS-CoV-2 inoculation. Data presented as mean SD (**p<0.01, nsp>0.05 otherwise, one-way ANOVA with Tukey's post-hoc test, n=8). (FIG. 5E) Antiviral cytokine interferon-gamma (IFN-7) expression from harvested mice lungs by RT-qPCR after treatment and SARS-CoV-2 inoculation. Data presented as mean SD (*p<0.05, nsp>0.05 otherwise, one-way ANOVA with Tukey's post-hoc test, n=8). (FIG. 5F) Hematoxylin and eosin (H&E) staining of harvested mice lung tissue sections following treatment and SARS-CoV-2 inoculation. Scale bars represent 50 m. (FIG. 5G) Pathology scoring of analyzed mouse lung tissue sections. Data presented as mean±SD (*p<0.05, **p<0.01, nsp>0.05 otherwise, one-way ANOVA with Tukey's post-hoc test, n=8). (FIG. 5H) Immunofluorescence staining of harvested mouse lung tissue sections after treatment and SARS-CoV-2 inoculation showing SARS-CoV-2 N protein (anti-SCV2 N protein antibody, green) and nucleus (DAPI, blue) signals. Scale bars represent 50 μm;

FIG. 6A, FIG. 6B, and FIG. 6C show the molecular design and characterization of Ligand PA. (FIG. 1A) Full peptide sequence (SEQ ID NO: 5) and chemical structure of Ligand, showing full sequence of ACE2-inhibiting peptide ligand, DX600, through which ACE2 specifically binds to the PA molecule. (FIG. 1B) Analytical RP-HPLC chromatogram of Ligand, showing high purity. (FIG. 1C) MALDI-ToF mass spectrum of Ligand, where peak at 4188.1 corresponds to [M+H]⁺ (compared to calculated molecular weight of 4186.82);

FIG. 7A, FIG. 7B, and FIG. 7C show the molecular design and characterization of Filler PA. (FIG. 7A) Full peptide sequence and chemical structure of Filler. (FIG. 7B) Analytical RP-HPLC chromatogram of Filler, showing high purity. (FIG. 7C) MALDI-ToF mass spectrum of Filler, where peaks at 834.8 and 850.9 correspond to [M+Na]⁺ and [M+K]⁺, respectively (compared to calculated molecular weight of 812.02);

FIG. 8A, FIG. 8B, and FIG. 8C show the self-assembly characterization of Ligand PA alone. (FIG. 8A) Representative transmission electron microscopy image of Ligand at 200 μM in MilliQ water (pH=7.4) after aging for 24 h, showing assembly into spherical aggregates. Diameter represented as mean±SD (n=35). (FIG. 8B) Dynamic light scattering measurement of Ligand at 100 μM in PBS (pH=7.4) after aging for 24 h, confirming presence of spherical aggregates observed with TEM. Diameter measurement presented as mean±SD (n=3). (FIG. 8C) Circular dichroism measurement of Ligand spherical aggregates at 100 μM in MilliQ water (pH=7.4) after aging for 24 h, showing random coil secondary structure as evidenced by negative peak around 200 nm;

FIG. 9A, FIG. 9B, FIG. 9C, and FIG. 9D show self-assembly characterization of the co-assembly of Filler and Ligand at varying molar ratios. Representative transmission electron microscopy (TEM) image of (FIG. 9A) Filler at 500 μM, in MilliQ water (pH=7.4) after aging for 24 h, showing assembly into ribbon-like filaments. Diameter represented as mean±SD (n=35). Representative TEM image of (FIG. 9B) 10:1, (FIG. 9C) 20:1, and (FIG. 9D) 50:1 molar ratio of Filler:Ligand with 50 μM Ligand in MilliQ water (pH=7.4) after aging for 24 h, showing co-assembly into ribbon-like filaments for every ratio studied. Diameters represented as mean±SD (n=35);

FIG. 10 is circular dichroism (CD) spectra of the co-assembly of Filler and Ligand at varying molar ratios, ranging from 1:0 to 50:1 Filler:Ligand. All spectra are representative of samples in MilliQ water at pH=7.4 after immediate dilution to 100 μM before scanning. The CD spectra of 1:0 shows an intense positive peak around 207 nm and red-shifted negative peak at ˜226 nm, which is suggestive of 3-turn character in addition to more linear 3-sheet character. Woody, R. W. in Peptides, polypeptides and proteins (eds E. R. Blout, F. A. Bovey, M. Goodman, & N. Lotan) 338-348 (John Wiley & Sons, Inc., 1974); Brahms et al., Identification of β,β-turns and unordered conformations in polypeptide chains by vacuum ultraviolet circular dichroism. Proceedings of the National Academy of Sciences 74, 3208-3212 (1977).

With increasing content of Ligand in the supramolecular structures, the β-turn content decreases and CD spectra represent more typical linear β-sheet character with stronger negative peak at ˜220 nm and weaker, blue-shifted positive peak at ˜202 nm. Spectrum for each sample represents average of 3 scans;

FIG. 11 shows the determination of critical micelle concentration (CMC) for Filler, Ligand, and their co-assembly in MilliQ water (pH=7.4) using Nile Red assay. The fluorescence intensity maximum is shifted from 660 nm to 635 nm as the Nile Red dye becomes encapsulated within the supramolecular structures of the PAs. The presented CMC values are calculated as the transition point between the two wavelengths. Data are presented as mean±SD (n=3);

FIG. 12A, FIG. 12B, FIG. 12C, FIG. 12D, FIG. 12E show the molecular design, characterization, and self-assembly behavior of sLigand PA. (FIG. 12A) Full peptide sequence and chemical structure of sLigand, showing scrambled sequence of ACE2-inhibiting peptide ligand, DX600, where loop is maintained in the same position, but the amino acid sequence was randomly shuffled otherwise. The sLigand structure comprises amino acid sequence VVVGKKGGOEGOEGOEGOEGYPKYPCTGSGSDGDELCGWYPHWPYR (SEQ ID NO: 7) (FIG. 12B) Analytical RP-HPLC chromatogram of sLigand, showing high purity. (FIG. 12C) MALDI-ToF mass spectrum of sLigand, where peak at 4187.7 corresponds to [M+H]⁺ (compared to calculated molecular weight of 4186.82). (FIG. 12D) Representative transmission electron microscopy image of sLigand at 200 μM in MilliQ water (pH=7.4) after aging for 24 h, showing assembly into spherical aggregates. Diameter represented as mean±SD (n=35). (FIG. 12E) Dynamic light scattering measurement of sLigand at 100 μM in PBS (pH=7.4) after aging for 24 h, confirming presence of spherical aggregates observed with TEM. Diameter measurement presented as mean±SD (n=3);

FIG. 13 shows the Zeta potential measurements of Filler alone and ACE2-docking filaments (20:1 molar ratio Filler:Ligand) before and after ACE2 addition. Filament zeta potential is negligibly impacted by incorporation of Ligand, emphasizing role of Filler in regulating surface charge. Large drop in zeta potential after addition of ACE2 suggests complexation at the surface of the supramolecular filaments. Data are presented as mean±SD (ns p>0.5, **p<0.01, ***p<0.001, one-way ANOVA with Tukey's post hoc test, n=3);

FIG. 14 shows the kinetic measurement of evolved fluorescence intensity of activity probe by ACE2 cleavage in the presence of ACE2-docking filaments. The concentration of Filler within the filaments is varied while holding Ligand and ACE2 concentration constant, showing greater inhibition of ACE2 with increased spacing. Data presented as mean±SD (n=3);

FIG. 15 shows the kinetic measurement of evolved fluorescence intensity of activity probe by ACE2 cleavage in the presence of ACE2-docking filaments. The molar ratio of Filler to Ligand is held constant (20:1) while the Ligand:enzyme ratio is varied, showing greater inhibition with greater Ligand to ACE2 ratios. Data presented as mean±SD (n=3);

FIG. 16 shows the kinetic measurement of evolved fluorescence intensity of activity probe by ACE2 cleavage in the presence of ACE2-docking filaments. The concentration of Filler and the Ligand:enzyme ratio are fixed while the concentration of Ligand is varied, showing greater inhibition of ACE2 with increasing Ligand concentration. Data presented as mean±SD (n=3);

FIG. 17A and FIG. 17B demonstrate the influence of incubation time on docking efficiency of ACE2 to ACE2-docking filament surfaces. Enzymatic inhibition (calculated based on relative reaction initial velocities compared to free ACE2) of ACE2 after incubation with ACE2-docking filaments for set time before assessment with activity assay, showing maximum docking achieved after 15 min. Data presented as mean±SD (***p<0.001, nsp>0.05 amongst groups 15 min and later; one-way ANOVA with Tukey's post hoc test, n=3);

FIG. 18 is circular dichroism (CD) spectra of fACE2 before and after nebulization (1 mM in PBS at pH=7.4 with immediate dilution to 100 μM before running sample), showing retention of β-sheet character (negative peaks around 220 nm) after nebulization but also a reduction in signal intensity reflective of disrupted hydrogen-bonding. Spectra represent average of 3 scans;

FIG. 19A, FIG. 19B, FIG. 19C, FIG. 19D, FIG. 19E, and FIG. 19F demonstrate post-nebulization filament fragmentation of various formulation concentrations of fACE2 in PBS (pH=7.4). Representative transmission electron microscopy images of fACE2 at (FIG. 19A) 200 μM, (FIG. 19B) 500 μM, and (FIG. 19C) 1 mM after nebulization, showing reduced filament length. Population size distributions for the contour lengths of observed filaments after jet nebulization for each formulation concentration of fACE2 (20 bins, 50 nm each) from TEM images, where represents average contour length given as mean±SD alongside median (M) measured length: (FIG. 19D) 200 μM (n=357 analyzed filaments), (FIG. 19E) 500 μM (n=386 analyzed filaments), and (FIG. 19F) 1 mM (n=369 analyzed filaments). Dotted lines represent fit of data to a Gaussian distribution. For average contour length of each starting concentration, nsp>0.05 by one-way ANOVA with Tukey's post hoc test, highlighting the similarity of the size distributions;

FIG. 20A, FIG. 20B, and FIG. 20C are release profiles of fACE2 from a jet nebulizer over the course of a 10 min nebulization event (500 μM formulation in PBS at pH=7.4). Release of fACE2 by (FIG. 20A) total mass emitted (g), (FIG. 20B) fraction of loaded dose (% by mass), and (FIG. 20C) weight of formulation fluid (% by mass), highlighting linear profile. Data presented as mean±SD (n=3); dashed lines represent results of linear regression analysis. For output rates of filament mass and fluid mass (%), nsp>0.05 by two-tailed unpaired t-test with Welch's correction, though slightly lower output rate of filaments may reflect heterogeneity in aerosol droplet concentration over the course of a nebulization event;

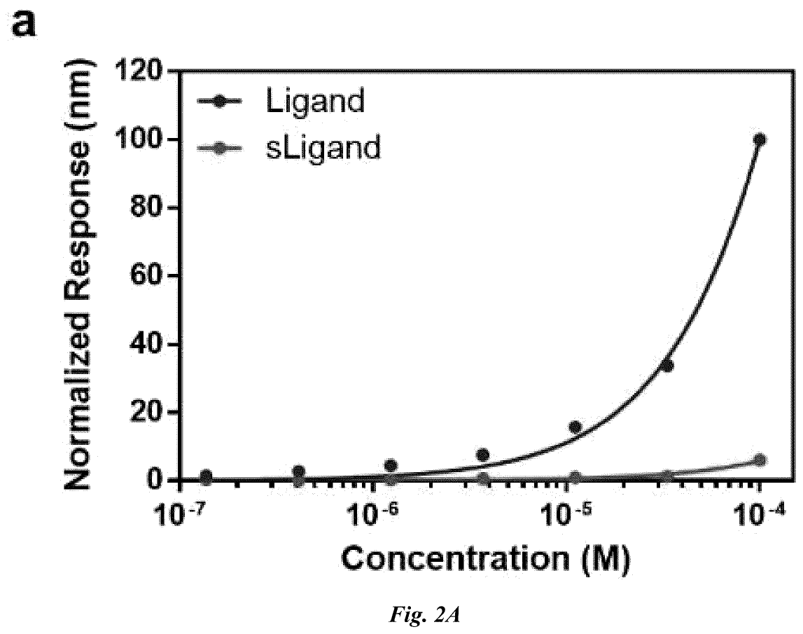

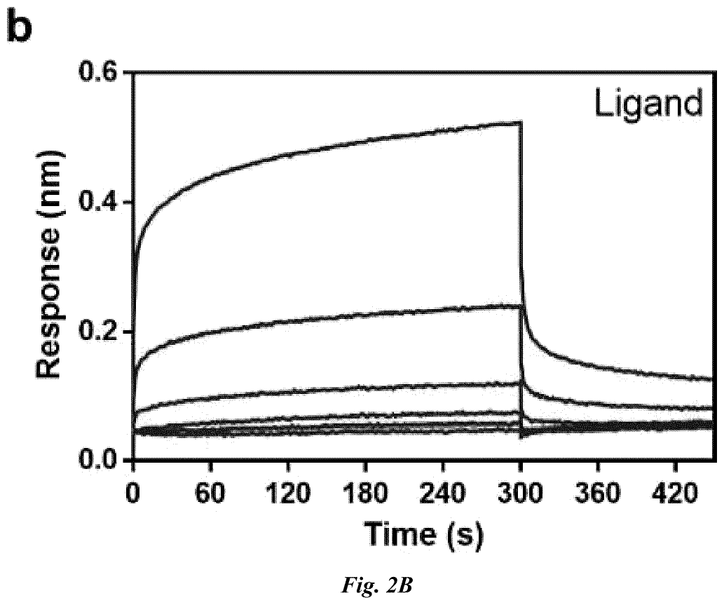

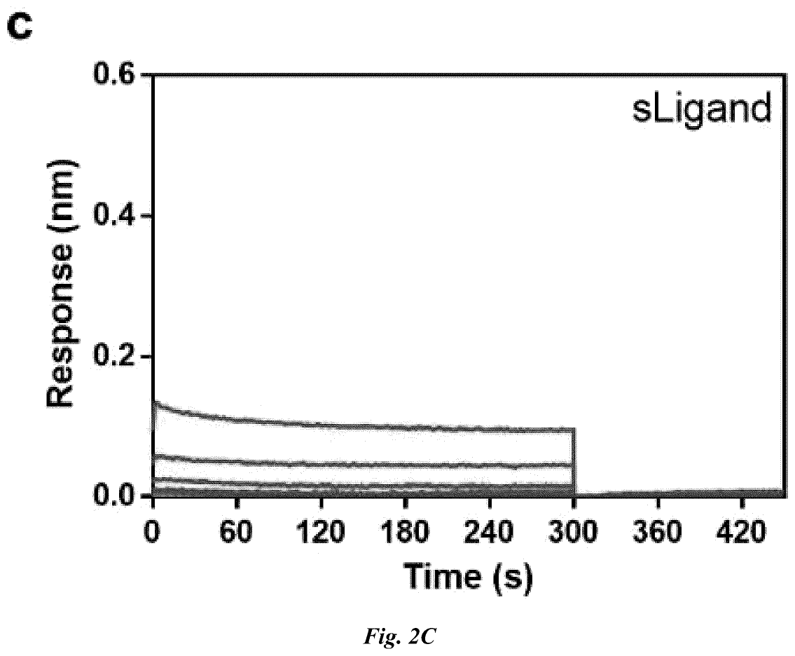

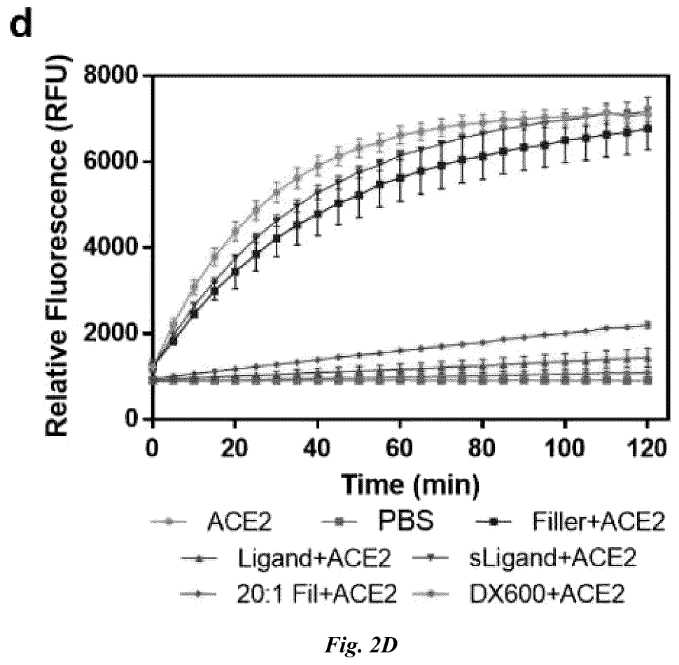

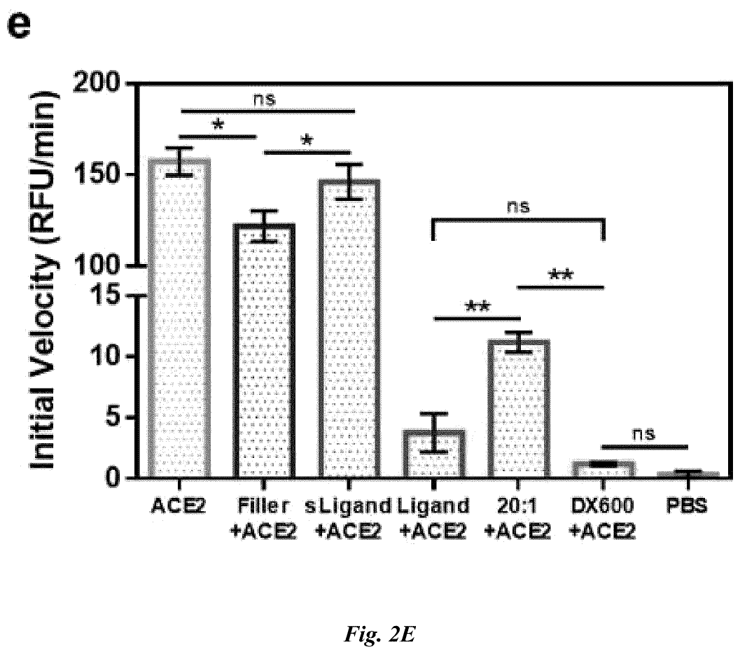

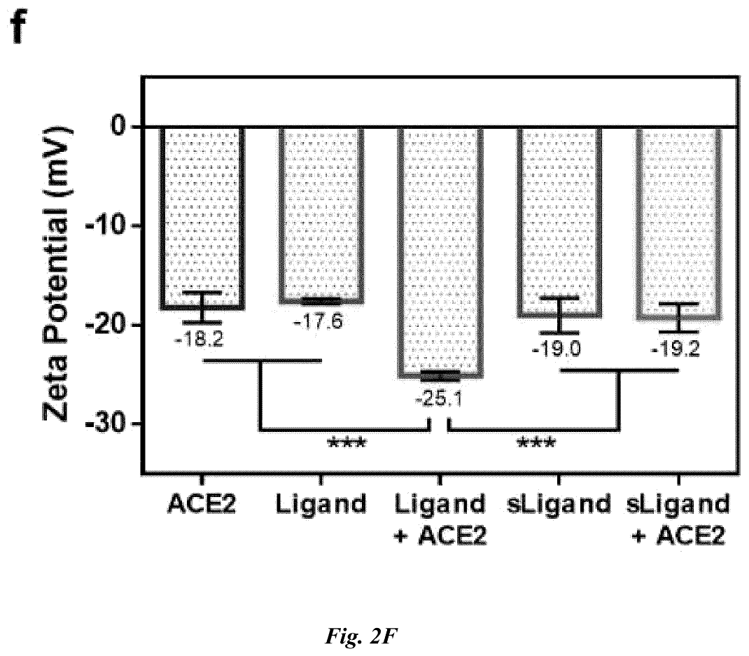

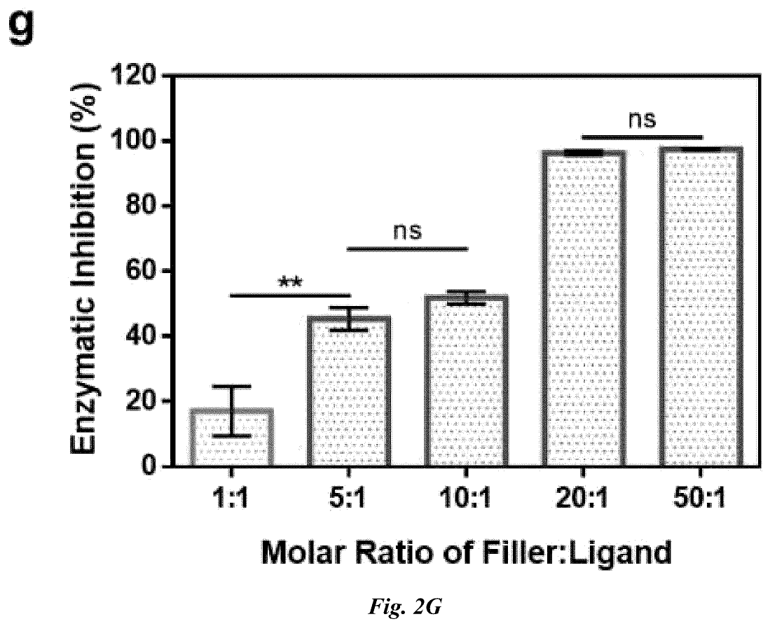

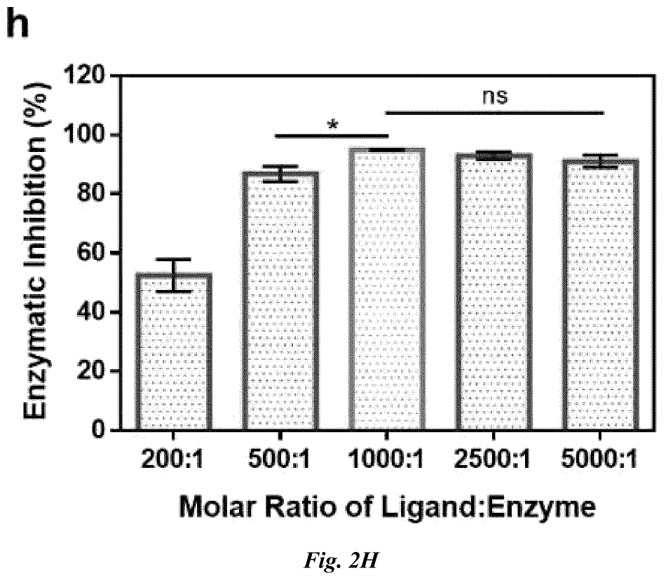

FIG. 2IA, FIG. 2IB, and FIG. 2IC demonstrate the cytotoxicity of ACE2-docking filaments after 48 h incubation concentrations (ranging from 0.1 to 100 μM) for (FIG. 2IA) NL20, (FIG. 2IB) A549, and (FIG. 2IC) 293/ACE2/TMPRSS2 cells lines as determined by MTT assay. Data presented as mean±SD (n=3 independent experiments);

FIG. 22A, FIG. 22B, and FIG. 22C show filament characterization of fACE2 after administration with an intranasal mucosal atomizer. Representative (FIG. 22A) low- and (FIG. 22B) high-magnification transmission electron microscopy images of fACE2 (500 μM in PBS, pH=7.4) post-atomization, showing instances of filament fragmentation and alignment, likely due to shear forces. (FIG. 22C) Circular dichroism (CD) spectra of fACE2 before and after atomization (500 μM in PBS at pH=7.4 with immediate dilution to 100 μM before running sample), showing retention of β-sheet character (negative peaks around 220 nm) after nebulization but also a reduction in signal intensity reflective of disrupted hydrogen-bonding. Spectra represent average of 3 scans;

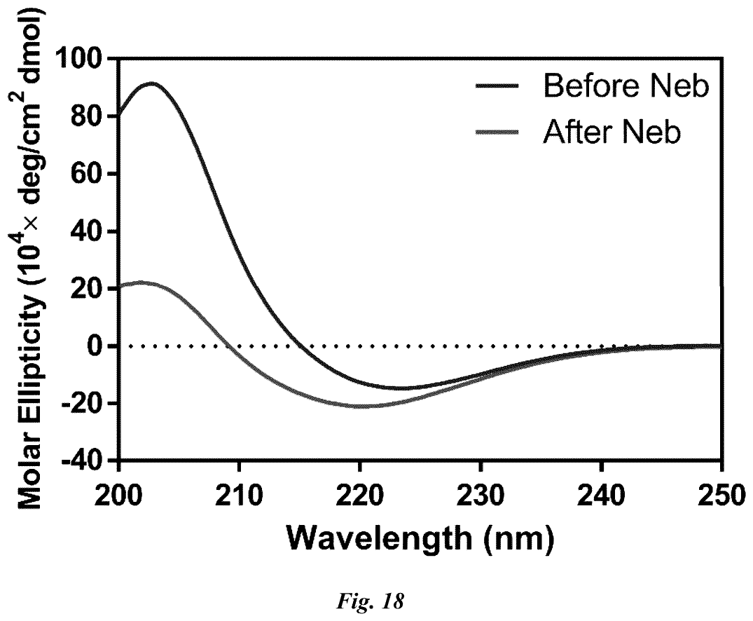

FIG. 23A and FIG. 18B are an in vivo lung distribution study of ACE2-docking filaments in K18-hACE2 mice after inhalation administration. (FIG. 23A) Representative transmission electron microscopy image of near-infrared dye Cyanine 5.5-loaded ACE2-docking filaments (500 μM in PBS, pH=7.4) used for visualizing filament distribution. (FIG. 23B) In Vivo Imaging System (IVIS) fluorescence imaging of K18-hACE2 mice after administration of atomized Cy5.5-loaded ACE2-docking filaments (in PBS, 10 nM equivalent ACE2 dose) either via intratracheal instillation or intranasal inhalation (control=intranasal inhalation of PBS only). Images on left shows retention of filaments in nasal cavity of mice 3 h post-administration. Lungs harvested from mice 24 hr post-administration of filaments shows strong fluorescence signal from filaments throughout lung tissues, suggesting enhanced retention (n=2 mice per group, 1 control). Experiments were repeated twice with similar results;

FIG. 24 shows the histology of lung tissue sections of K18-hACE2 mice 24 hr after administration of Cy5.5-loaded ACE2-docking filaments (in PBS, 10 nM ACE2 equivalent dose) via intranasal inhalation. Compared to PBS-only control, hematoxylin and eosin (H&E) staining and immunofluorescence show no obvious sign of lung cell apoptosis, inflammation, or neutrophil infiltration from treatment with ACE2-docking filaments, suggesting filaments are relatively biocompatible and safe delivery vehicles for ACE2 (neutrophil invasion, myeloperoxidase (anti-MPO antibody), red; apoptosis marker, cleaved-caspase 3 (anti-CC3 antibody), red; pro-inflammatory cytokine production, inducible nitric oxide synthase (anti-iNOS antibody), green; cell nuclei, DAPI, blue). Experiments were repeated twice with similar results;

FIG. 25 is the immunofluorescence staining of harvested mouse lung tissue sections after treatment and SARS-CoV-2 inoculation, assessing neutrophil infiltration. Neutrophil invasion is markedly decreased with treatment with fACE2, indicating alleviated lung inflammation and related pathology (myeloperoxidase (anti-MPO antibody), red; cell nuclei (DAPI), blue). Scale bars represent 50 m; and

FIG. 26 is the full chemical structure of the Ligand PA design.

### BRIEF DESCRIPTION OF THE SEQUENCE LISTING

SEQ ID NO: 1 represents the amino acid sequence VVVGEE.

SEQ ID NO: 2 represents the amino acid sequence VVVGKK.

SEQ ID NO: 3 represents the amino acid sequence VVV.

SEQ ID NO: 4 represents the amino acid sequence VVVGKKGGOEGOEGOEGOEG (SEQ ID NO: 4).

SEQ ID NO: 5 represents the amino acid sequence GDYSHCSPLRYYPWWKCTYPDPEGGG.

SEQ ID NO: 6 represents the amino acid sequence VVVGKKGGOEGOEGOEGOEGGDYSHCSPLRYYPWWKCTYPDPEGGG.

SEQ ID NO. 7 represents the amino acid sequence VVVGKKGGOEGOEGOEGOEGYPKYPCTGSGSDGDELCGWYPHWPYR.

SEQ ID NO: 8 represents the amino acid sequence YPKYPCTGSGSDGDELCGWYPHWPYR.

SEQ ID NO: 9 represents the amino acid sequence GSRIGCRDSRCNWWAPGEGGG.

SEQ ID NO: 10 represents the amino acid sequence GSRGFCRDSSCSFPAPGEGGG.

SEQ ID NO: 11 represents the amino acid sequence AGWEVCHWAPMMCKHGGTEGGG.

SEQ ID NO: 12 represents the amino acid sequence AGSDWCGTWNNPCFHQGTEGGG.

SEQ ID NO: 13 represents the amino acid sequence GDRLHCKPQRQSPWMKCQHLDPEGGG.

SEQ ID NO: 14 represents the amino acid sequence GDLHACRPVRGDPWWACTLGDPEGGG.

SEQ ID NO: 15 represents the amino acid sequence GDRYLCLPQRDKPWKFCNWFDPEGGG.

SEQ ID NO: 16 represents the amino acid sequence GDYSHCSPLRYYPWWKCTYPDPEGGG.

SEQ ID NO: 17 represents the amino acid sequence GDGFTCSPIRMFPWFRCDLGDPEGGG.

SEQ ID NO: 18 represents the amino acid sequence GDFSPCKALRHSPWWVCPSGDPEGGG.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Unless otherwise defined, all technical and scientific terms used herein have the same meaning as commonly understood by one of ordinary skill in the art to which this invention belongs. In case of conflict, however, the present specification, including definitions, will control. Accordingly, in the context of the embodiments described herein, the following definitions apply.

The term “amino acid” refers to natural amino acids, unnatural amino acids, and amino acid analogs, all in their D and L stereoisomers, unless otherwise indicated, if their structures allow such stereoisomeric forms Natural amino acids include alanine (Ala. or A), arginine (Arg or R), asparagine (Asn or N), aspartic acid (Asp or D), cysteine (Cys or C), glutamine (Gin or Q), glutamic acid (Glu or E), glycine (Gly or G), histidine (His or H), isoleucine (lie or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2-diaminopimelic acid, 2,3-dianinopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”) isodesmosine, allo-isoleucine, N-methylalanine (“MeAla” or “Nine”), N-allylglycine (“NA G”) including N-methylglycine, N-methylisoleucine, N-alkylpentylglycine (“NAPG”) including N-methylpentylglycine N-methylvaline, naphthylalaniie, norvaline (“Norval”), norleucine (“Norleu”), octylglycine (“OctG”), ornithine (“Orn”), pentylglycine (“pG” or “PGly”), pipecolic acid, tlioproline (“ThioP” or “tPro”), homoLysine (“bLys”), and homnoArginine (“hArg”). The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “peptide” refers a short polymer of amino acids linked together by peptide bonds. In contrast to other amino acid polymers (e.g., proteins, polypeptides, and the like), peptides are of about 50 amino acids or less in length. A peptide may comprise natural amino acids, non-natural amino acids, amino acid analogs, and/or modified amino acids. A peptide may be a subsequence of naturally occurring protein or a non-natural (artificial) sequence.

As used herein, the term “artificial” refers to compositions and systems that are designed or prepared by man, and are not naturally occurring. For example, an artificial peptide or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (F);
3) Asparagine (N) and Glutamine (Q);
4) Arginine (R) and Lysine (K);
5) Isoleucine (1), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)): polar neutral (serine (S), threonine (T), asparagine (N), glutamine (Q)); non-polar aliphatic (alanine (A), valine (V), leucine (L), isoleucine (1), methionine (m)); and non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, and the like) have the same sequential composition of monomer subunits. The term “sequence similarity” refers to the degree with which two polymer sequences (e.g., peptide, polypeptide, nucleic acid, and the like) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, and the like), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence.

As used herein, the term “nanofiber” refers to an elongated or threadlike filament (e.g., having a significantly greater length dimension that width or diameter) with a diameter typically less than 100 nanometers.

As used herein, the term “supramolecular” (e.g., “supramolecular complex,” “supramolecular interactions,” “supramolecular fiber,” “supramolecular polymer,” and the like) refers to the non-covalent interactions between molecules (e.g., polymers, macromolecules, and the like) and the multicomponent assemblies, complexes, systems, and/or fibers that form as a result.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, and the like. In some aspects, a wound dressing or coating comprises hydrolyzable ester linkages that provide the biodegradability.

As used herein, the term “physiological conditions” refers to the range of conditions of temperature, pH and tonicity (or osmolality) normally encountered within tissues in the body of a living human.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components.

As used herein, the term “peptide amphiphile” refers to a molecule that, at a minimum, includes a non-peptide hydrophobic segment, a structural peptide segment and optionally a functional peptide segment. The peptide amphiphile may express a net charge at physiological pH, either a net positive or negative net charge, or may be zwitterionic (i.e., carrying both positive and negative charges).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±20%, in some embodiments 10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

In some aspects, the presently disclosed subject matter provides the use of soluble angiotensin-converting enzyme 2 (ACE2) or molecules of related functions for preventing and treating viral infections, such as a coronavirus infection. It is generally accepted that membrane-bound ACE2 serves as the key receptor for coronavirus entry into cells. At the same time, soluble ACE2 plays a key role in regulating the activities of immune systems. The presently disclosed subject matter, in part, describes the use of soluble ACE2 to both modulate the activities of immune systems to battle with coronavirus and prevent viral entry into cells.

In one embodiment, the presently disclosed subject matter provides self-assembling molecules that are capable of forming supramolecular structures in aqueous solution. These supramolecular structures are able to immobilize ACE2 or to be applied in the preventative, i.e., prophylactic, treatment of coronaviruses, such as SARS. In particular embodiments, the presently disclosed self-assembling molecules include an ACE2-docking segment that binds ACE2 through its enzymatic site, thereby allowing for affinity binding of coronavirus at its exposed S-protein docking site (the site through which coronavirus enters the epithelial cells of the lung and causes infection). The ACE2-docking segment could be either an activator or inhibitor for the ACE2's enzymatic activities that regulate the immune system.

The presently disclosed supramolecular nanostructures loaded with ACE2 are capable of effectively traveling through aerosols. This property allows for their inhalable delivery or use in a nasal spray. Inhaled nanostructures can deliver ACE2 enzyme on their surface directly to the lungs. Further, through rational design, the constructs can be captured within the mucus layer (through charge interactions) and present ACE2 over the lining of the lungs' epithelia. The presented ACE2 can capture inhaled coronavirus prematurely with strong binding to the virus, which allows for subsequent clearing and degradation of the virus by the lungs' clearance mechanisms (macrophages, mucociliary escalator) and prevent infection.

In one particular embodiment, filamentous nanostructures could be created such that the filamentous shape can enhance the deposition and retention of the filaments within the lungs. The use of peptide-based conjugate systems renders the system biodegradable in nature. The function of the inhalable ACE2-docking filaments is to prematurely capture inhaled coronavirus and prevent potential infection.

Accordingly, the presently disclosed subject matter provides a supramolecular filament comprising: an ACE2-binding ligand peptide amphiphile (PA) comprising, in order from N-terminus to C-terminus: i) an aliphatic segment, ii) an intermolecular interaction-regulating peptide segment, iii) a hydrophilic spacer, and iv) an ACE2 inhibitor, wherein the intermolecular interaction-regulating peptide segment of the ACE2-binding ligand peptide comprises, in order from N-terminus to C-terminus: a hydrogen bond-contributing sequence and one or more charged amino acid residues; a self-assembling filler peptide amphiphile (PA) comprising, in order from N-terminus to C-terminus: i) an aliphatic segment, and ii) an intermolecular interaction-regulating peptide segment, wherein the intermolecular interaction-regulating peptide segment of the self-assembling filler PA comprises, in order from N-terminus to C-terminus: a hydrogen bond-contributing sequence and one or more charged amino acid residues that have an opposite charge to the charged amino acid residues of the ACE2-binding ligand PA; and a soluble ACE2 comprising an S-protein binding site and a carboxypeptidase active site, wherein the soluble ACE2 is bound at the carboxypeptidase active site to the ACE2 inhibitor.

The segments of the ACE2-binding ligand PA are covalently attached. For example, each in order from N-terminus to C-terminus: the aliphatic segment is covalently attached to the intermolecular interaction-regulating peptide segment, which is covalently attached to the hydrophilic spacer, which is covalently attached the ACE2 inhibitor) and, for example, in order from N-terminus to C-terminus: the one or more charged amino acid residues and a hydrogen bond-contributing sequence of the intermolecular interaction-regulating peptide segment are covalently attached. Spacers (for example, 1-2 GG residues) may be included between segments and covalently attached; or other intermediate sequences or structures may be included and covalently attached.

The segments of the self-assembling filler PA are covalently attached. For example, each in order from N-terminus to C-terminus: the aliphatic segment is covalently attached to the intermolecular interaction-regulating peptide segment) and, for example, in order from N-terminus to C-terminus: the one or more charged amino acid residues and a hydrogen bond-contributing sequence of the intermolecular interaction-regulating peptide segment are covalently attached. Spacers (for example, 1-2 GG residues) may be included between segments and covalently attached; or other intermediate sequences or structures may be included and covalently attached.

In some aspects, the peptide amphiphile (PA) molecules described herein are synthesized using preparatory techniques well-known to those skilled in the art, for example, by standard solid-phase peptide synthesis. Synthesis typically starts from the C-terminus, to which amino acids are sequentially added, for example, using either a Rink amide resin (resulting in an —NH₂ group at the C-terminus of the peptide after cleavage from the resin), or a Wang resin (resulting in an —OH group at the C-terminus).

In some embodiments, the aliphatic segment is incorporated at the N-terminus of the peptide after the last amino acid coupling, and is linked to the N-terminal amino acid through an acyl bond. In some embodiments the aliphatic segment is a hydrophobic segment. In aqueous solutions, PA molecules self-assemble (e.g., into cylindrical micelles (a.k.a nanofibers)) that bury the hydrophobic segment in their core and display a functional peptide on the surface.

In certain embodiments, the aliphatic segment (e.g., hydrocarbon and/or alkyl tail) segment of sufficient length (e.g., >3 carbons, >5 carbons, >7 carbons, >9 carbons, and the like, that is 3, 4, 5, 6, 7, 8, 9, 10 carbon atoms) is covalently coupled to a peptide segment. The aliphatic segment should be of a sufficient length to provide amphiphilic behavior and micelle (or nanosphere or nanofiber) formation in water or another polar solvent system. In some embodiments, the aliphatic segment comprises an alkyl chain (e.g., saturated) of 4-25 carbons (e.g., 4, 5, 6, 7, 8, 9, 10, 11, 12, 13, 14, 15, 16, 17, 18, 19, 20, 21, 22, 23, 24, 25), fluorinated segments, or fluorinated alkyl tails. In some embodiments, the aliphatic segment comprises an acyl chain of the formula: Cₙ₋₁H_{2n-1C}(O) (e.g., saturated) with 4-25 carbons (e g., 4, 5, 6, 7, 8, 9, 10, 11, 12, 13, 14, 15, 16, 17, 18, 19, 20, 21, 22, 23, 24, 25). In some embodiments, the aliphatic segment of the ACE2-binding ligand PA or the self-assembling filler PA comprises a dodecyl chain.

In some embodiments, a plurality of such PAs will self-assemble in water (or aqueous solution) into a nanostructure (e g., nanofiber). In various embodiments, the relative lengths of the peptide segment and aliphatic segment result in differing PA molecular shape and nanostructural architecture. For example, a broader peptide segment and narrower hydrophobic segment results in a generally conical molecular shape that has an effect on the assembly of PAs (See, e.g., J. N. Israelachvili Intermolecular and surface forces; 2nd ed.; Academic: London San Diego, 1992; herein incorporated by reference in its entirety) Other molecular shapes have similar effects on assembly and nanostructural architecture. In various embodiments, aliphatic segments pack in the center of the assembly with the peptide segments exposed to an aqueous or hydrophilic environment to form cylindrical nanostructures that resemble filaments. Such nanofilamems display the peptide regions on their exterior and have a hydrophobic core.

The ACE2-binding ligand PA comprises a hydrophilic spacer and an ACE2 inhibitor at the C-terminus of the PA. The hydrophilic spacer is attached to the C-terminal end of the intermolecular interaction-regulating peptide segment of the ACE2-binding ligand PA, and the ACE2 inhibitor is attached to the C-terminal end of the hydrophilic spacer. In some embodiments, the hydrophilic spacer comprises hydrophilic amino acids (e.g., R, N, D, Q, E, K). In some embodiments, the hydrophilic spacer comprises hydrophilic amino acids (e.g. R, N, D, Q, E, K) and neutral amino acids (e.g., G, H, P, S, T, or Y). In some embodiments, the hydrophilic spacer comprises a hydrophilic oligoethylene glycol (OEG) chain having between about 4 to 16 OEG units. In some embodiments, the OEG chain has a length selected from 4, 8, 10, 12, 14, and 16 units. In some embodiments, the OEG chain comprises 4 units. In some embodiments, the hydrophilic spacer further comprises a double glycine (GG) sequence.

The ACE2 inhibitor is a functional moiety of the ACE2-binding ligand PA that is displayed by the ACE2-binding ligand PA. In some embodiments, the ACE2 inhibitor is DX600. In some embodiments, the ACE2 inhibitor comprises a chemical structure of:

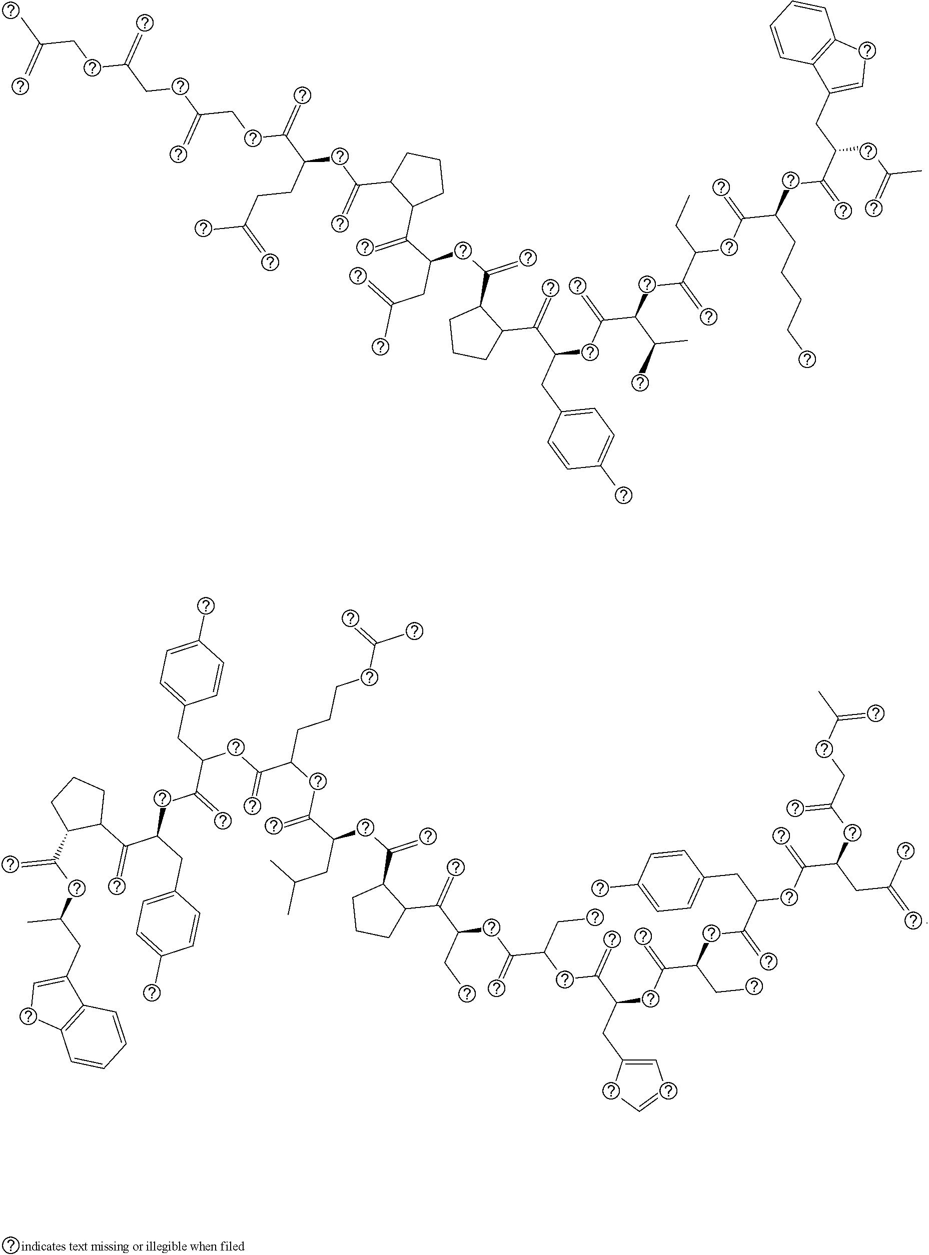

In some embodiments, the ACE2 inhibitor is MLN-4760. In some embodiments, the ACE2 inhibitor comprises the chemical structure of:

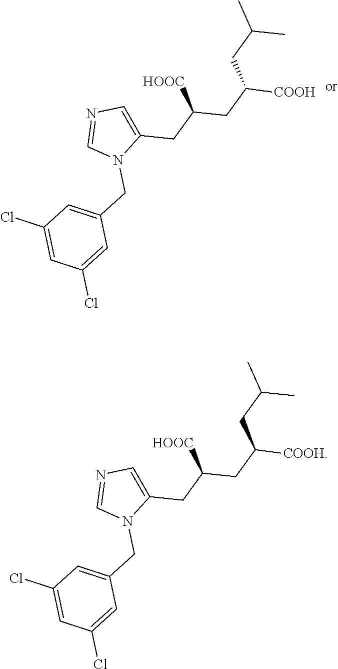 [SMILES: CC(C)C[C@@H](C[C@@H](Cc1cncn1Cc1cc(Cl)cc(Cl)c1)C(=O)O)C(=O)O ; CC(C)C[C@H](C[C@@H](Cc1cncn1Cc1cc(Cl)cc(Cl)c1)C(=O)O)C(=O)O]

In some embodiments, the ACE2 inhibitor comprises an amino acid sequence of GDYSHCSPLRYYPWWKCTYPDPEGGG (SEQ ID NO:5) or functional analogues thereof. In some embodiments, the ACE2 inhibitor comprises at least 70% sequence similarity (e.g., 8, 7, 6, 5, 4, 3, 2, 1, or 0 (or ranges there between) non-conservative substitutions) with SEQ ID NO:5. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO:5 to bind to soluble ACE2. In some embodiments, the ACE2 inhibitor has enhanced binding affinity for soluble ACE2 compared to SEQ ID NO:5 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of GSRIGCRDSRCNWWAPGEGGG (SEQ ID NO: 9) (DX524) or functional analogues thereof. In some embodiments, the ACE2 inhibitor comprises at least 70% sequence similarity (e.g., 6, 5, 4, 3, 2, 1, or 0 (or ranges there between) non-conservative substitutions) with SEQ ID NO: 9. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 9 to bind to soluble ACE2. In some embodiments, the ACE2 inhibitor has enhanced binding affinity for soluble ACE2 compared to SEQ ID NO: 9 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of GSRGFCRDSSCSFPAPGEGGG (SEQ ID NO: 10) (DX525) or functional analogues thereof. In some embodiments, the ACE2 inhibitor comprises at least 70% sequence similarity (e.g., 6, 5, 4, 3, 2, 1, or 0 (or ranges there between) non-conservative substitutions) with SEQ ID NO: 10. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 10 to bind to soluble ACE2. In some embodiments, the ACE2 inhibitor has enhanced binding affinity for soluble ACE2 compared to SEQ ID NO: 10 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of AGWEVCHWAPMMCKHGGTEGGG (SEQ ID NO: 11) (DX529) or functional analogues thereof. In some embodiments, the ACE2 inhibitor comprises at least 70% sequence similarity (e.g., 6, 5, 4, 3, 2, 1, or 0 (or ranges there between) non-conservative substitutions) with SEQ ID NO: 11. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 11 to bind to soluble ACE2. In some embodiments, the ACE2 inhibitor has enhanced binding affinity for soluble ACE2 compared to SEQ ID NO: 11 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of AGSDWCGTWNNPCFHQGTEGGG (SEQ ID NO: 12) (DX531) or functional analogues thereof. In some embodiments, the ACE2 inhibitor comprises at least 70% sequence similarity (e.g., 6, 5, 4, 3, 2, 1, or 0 (or ranges there between) non-conservative substitutions) with SEQ ID NO: 12. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 12 to bind to soluble ACE2. In some embodiments, the ACE2 inhibitor has enhanced binding affinity for soluble ACE2 compared to SEQ ID NO: 12 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of GDRLHCKPQRQSPWMKCQHLDPEGGG (SEQ ID NO: 13) (DX512) or functional analogues thereof. In some embodiments, the ACE2 inhibitor comprises at least 70% sequence similarity (e.g. 7, 6, 5, 4, 3, 2, 1, or 0 (or ranges there between) non-conservative substitutions) with SEQ ID NO: 13. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 13 to bind to soluble ACE2. In some embodiments, the ACE2 inhibitor has enhanced binding affinity for soluble ACE2 compared to SEQ ID NO: 13 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of GDLHACRPVRGDPWWACTLGDPEGGG (SEQ ID NO: 14) (DX513) or functional analogues thereof. In some embodiments, the ACE2 inhibitor comprises at least 70% sequence similarity (e.g. 7, 6, 5, 4, 3, 2, 1, or 0 (or ranges there between) non-conservative substitutions) with SEQ ID NO: 14. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 14 to bind to soluble ACE2. In some embodiments, the ACE2 inhibitor has enhanced binding affinity for soluble ACE2 compared to SEQ ID NO: 1 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of GDRYLCLPQRDKPWKFCNWFDPEGGG (SEQ ID NO: 15) (DX599) or functional analogues thereof. In some embodiments, the ACE2 inhibitor comprises at least 70% sequence similarity (e.g. 7, 6, 5, 4, 3, 2, 1, or 0 (or ranges there between) non-conservative substitutions) with SEQ ID NO: 15. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 15 to bind to soluble ACE2. In some embodiments, the ACE2 inhibitor has enhanced binding affinity for soluble ACE2 compared to SEQ ID NO: 15 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of GDYSHCSPLRYYPWWKCTYPDPEGGG (SEQ ID NO: 16) (DX600) or functional analogues thereof. In some embodiments, the ACE2 inhibitor comprises at least 70% sequence similarity (e.g. 7, 6, 5, 4, 3, 2, 1, or 0 (or ranges there between) non-conservative substitutions) with SEQ ID NO: 16. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 16 to bind to soluble ACE2. In some embodiments, the ACE2 inhibitor has enhanced binding affinity for soluble ACE2 compared to SEQ ID NO: 16 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of GDGFTCSPIRMFPWFRCDLGDPEGGG (SEQ ID NO: 17) (DX601) or functional analogues thereof. In some embodiments, the ACE2 inhibitor comprises at least 70% sequence similarity (e.g. 7, 6, 5, 4, 3, 2, 1, or 0 (or ranges there between) non-conservative substitutions) with SEQ ID NO: 17. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 17 to bind to soluble ACE2. In some embodiments, the ACE2 inhibitor has enhanced binding affinity for soluble ACE2 compared to SEQ ID NO: 17 (e.g., 1.1-fold, 1.5-fold, 2-fold, 3-fold, 4-fold, 5-fold, 6-fold, 7-fold, 8-fold, 9-fold, 10-fold, or more, or ranges there between).

In some embodiments, the ACE2 inhibitor comprises an amino acid sequence of GDFSPCKALRHSPWWVCPSGDPEGGG (SEQ ID NO: 18) (DX602) or functional analogues thereof. In some embodiments, the ACE2 inhibitor comprises at least 70% sequence similarity (e.g. 7, 6, 5, 4, 3, 2, 1, or 0 (or ranges there between) non-conservative substitutions) with SEQ ID NO: 18. In some embodiments, the ACE2 inhibitor retains a substantial degree (e.g., 10%, 20%, 30%, 40%, 50%, 60%, 70%, 80%, 90%, 100%, or ranges there between) of the capacity of SEQ ID NO: 18 to bind to soluble ACE2. In some embodiments, the ACE2 inhibitor has enhanced binding affinity for soluble ACE2 compared to SEQ ID NO: 18 (e.g., 1.1-fold, 1.5-fold, 2-fold, 3-fold, 4-fold, 5-fold, 6-fold, 7-fold, 8-fold, 9-fold, 10-fold, or more, or ranges there between).

The intermolecular interaction-regulating peptide segment of the ACE2-binding ligand peptide comprises one or more charged amino acid residues and a hydrogen bond-contributing sequence; and the intermolecular interaction-regulating peptide segment of the self-assembling filler PA comprises a hydrogen bond-contributing sequence and one or more charged amino acid residues that have an opposite charge to the charged amino acid residues of the ACE2-binding ligand PA. In some embodiments, negatively charged amino acids at pH 7-7.4 are aspartic acid or aspartate (D), glutamic acid or glutamate (E). In some embodiments, positively charged amino acids at pH 7-7.4 are Arginine (R), Histidine (H), and Lysine (K).

In some embodiments, the charged amino acid residues of the self-assembling filler PA comprises: (i) one or more negatively charged glutamic acid residues; or (ii) one or more positively charged lysine residues. In some embodiments, the charged amino acid residues of the ACE2-binding ligand PA comprises one or more positively charged lysine residues and the charged amino acid residues of the self-assembling filler PA comprises one or more negatively charged glutamic acid residues. In some embodiments, the self-assembling filler PA and the ACE2-binding ligand PA form a filamentous structure comprising electrostatic complexation between the charged amino acid residues of the ACE2-binding ligand PA and the charged amino acid residues of the self-assembling filler PA.

In some embodiments, the hydrogen bond-contributing sequence of the self-assembly filler PA comprises a sequence of VVV (SEQ ID NO: 3). In some embodiments, the hydrogen bond-contributing sequence of the self-assembly filler PA comprises a sequence of 3-10 amino acid residues selected from: Met (M Val (V), Ile (I), Cys (C), Tyr (Y), Phe (F), Gln (Q), Leu (L), Thr (T), Ala (A), and Gly (G) In some embodiments, the hydrogen bond-contributing sequence of the ACE2-binding ligand PA comprises a sequence of VVV (SEQ ID NO. 3). In some embodiments, the hydrogen bond-contributing sequence of the ACE2-binding ligand PA comprises a sequence of 3-10 amino acid residues selected from: Met (M), Val (V), Ile (I), Cys (C), Tyr (Y), Phe (F), Gln (Q), Leu (L), Thr (T), Ala (A), and Gly (G), In some embodiments, the hydrogen bond-contributing sequence of the self-assembly filler PA comprises 3-10 amino acid residues selected from amino acids with an —OH group, —CONH₂ group, or —NH₂ group; and the hydrogen bond-contributing sequence of the ACE2-binding ligand PA comprises 3-10 amino acid residues selected from amino acids with an —OH group, —CONH₂ group, or —NH₂ group. In some embodiments, the hydrogen bond-contributing sequence of the self-assembly filler PA comprises 3-10 amino acid residues selected from amino acids with an —OH group, —COOH group, or —CONH₂ group; and the hydrogen bond-contributing sequence of the ACE2-binding ligand PA comprises 3-10 amino acid residues selected from amino acids with an —OH group, —COOH group, or —CONH₂ group. In some embodiments, the self-assembling filler PA and the ACE2-binding ligand PA form a filamentous structure comprising a hydrogen bonding network.

In some embodiments, the intermolecular interaction-regulating peptide segment of the ACE2-binding ligand PA comprises a sequence of VVVGKK (SEQ ID NO: 2) and the intermolecular interaction-regulating peptide segment of the self-assembling filler PA comprises a sequence of VVVGEE (SEQ ID NO: 1). In some embodiments, the intermolecular interaction-regulating peptide segment of the self-assembling filler PA comprises a sequence of VVVGEE (SEQ ID NO: 1).

In some embodiments, the self-assembling filler PA comprises the following chemical structure:

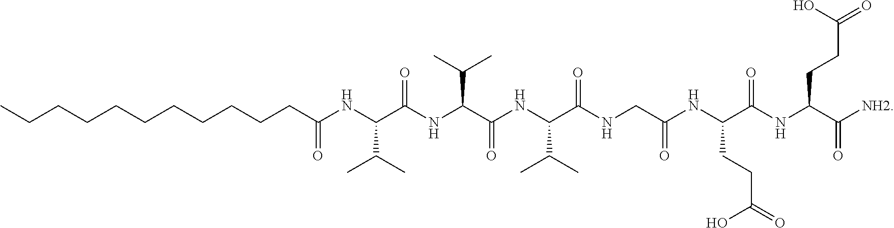 [SMILES: CCCCCCCCCCCC(=O)N[C@H](C(=O)N[C@H](C(=O)N[C@H](C(=O)NCC(=O)N[C@@H](CCC(=O)O)C(=O)N[C@@H](CCC(=O)O)C(N)=O)C(C)C)C(C)C)C(C)C]

In some embodiments, the ACE2-binding ligand PA comprises amino acid VVVGKKGGOEGOEGOEGOEGGDYSHCSPLRYYPWWKCTYPDPEGGG (SEQ ID NO: 6). In some embodiments, the ACE2-binding ligand PA comprises the following structure:

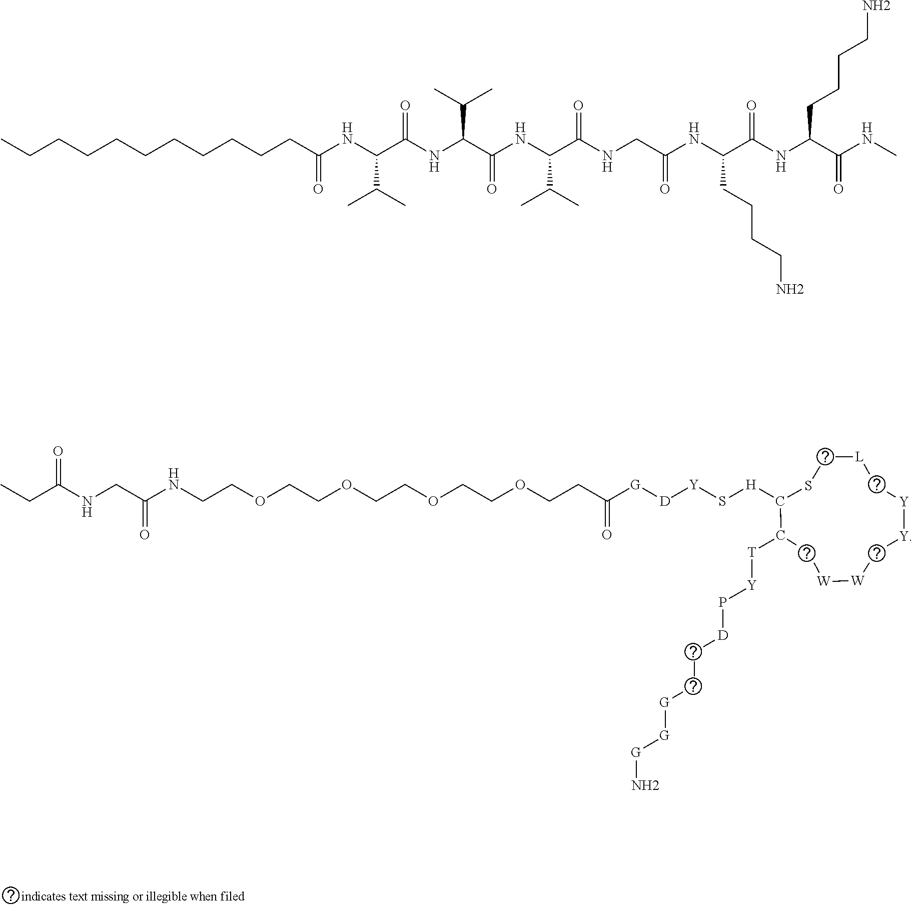

In some embodiments, the ACE2-binding ligand PA has the following chemical structure:

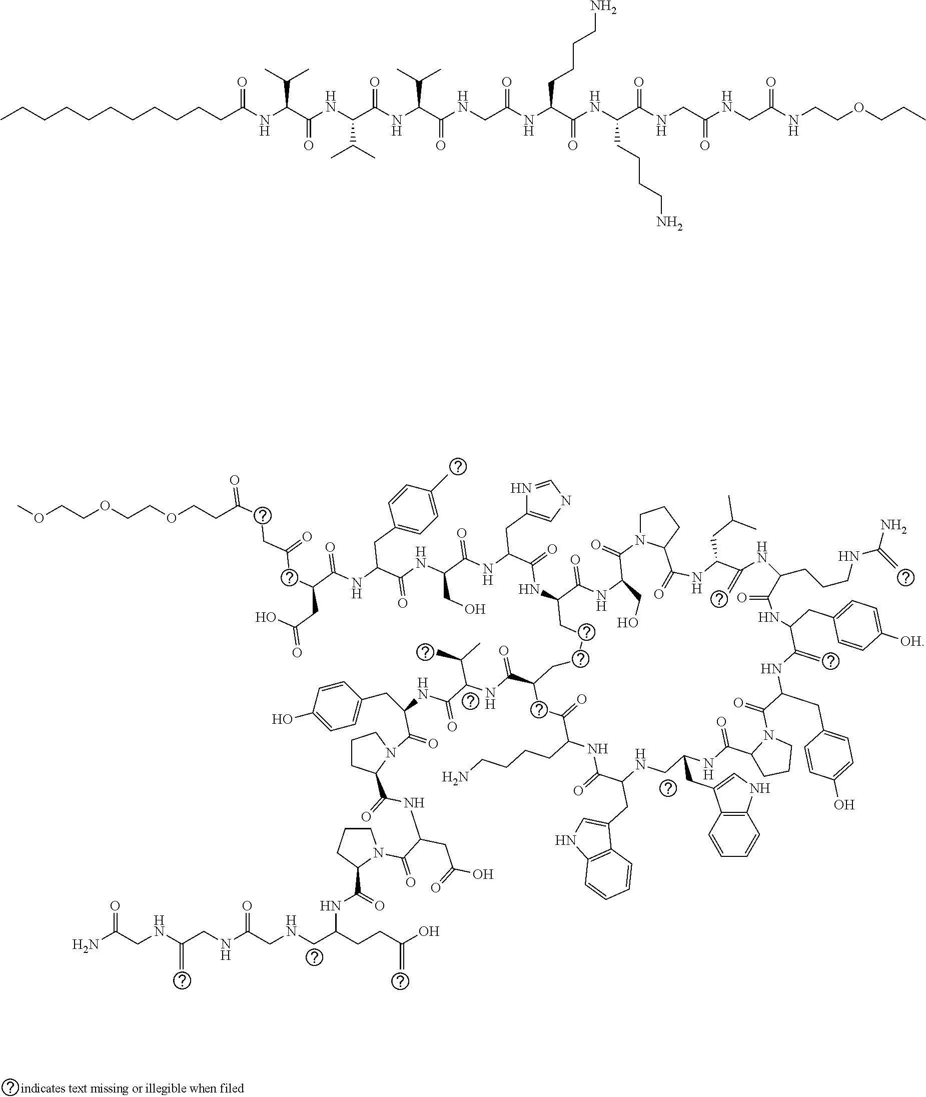

In some embodiments, the supramolecular filament comprises a molar ratio of the self-assembling filler PA to the ACE2-binding ligand PA in the range of about 10:1 to about 50:1, including 10:1, 15:1, 20:1, 25:1, 30:1, 35:1, 40:1, 45:1, and 50:1. In some embodiments, the molar ratio of filler to ligand is about 20:1.

In some embodiments, the supramolecular filament is biodegradable. In some embodiments, the supramolecular filament has a shape adapted to enhance deposition and retention of the filament within a lung of a subject.

In some embodiments, the presently disclosed subject matter provides a composition comprising the supramolecular filament described herein. In other embodiments, the presently disclosed subject matter provides a pharmaceutical formulation comprising the presently disclosed supramolecular filament and a pharmaceutically acceptable carrier. In other embodiments, the presently disclosed subject matter provides a kit comprising the presently disclosed supramolecular filament, pharmaceutical formulation, and/or respirable aerosol or nasal spray. The kit may include instructions for use, buffers, reagents, or other components to facilitate the mode of administration. The kit may include materials to facilitate nasal administration. The kit may include an inhaler, such as a metered dose inhaler known in the art. The information and instructions may be in the form of words, pictures, or both, and the like. In addition or in the alternative, the kit may include the medicament, a composition, or both; and information, instructions, or both, regarding methods of application of medicament, or of composition, preferably with the benefit of treating or preventing medical conditions in mammals (e.g., humans).

In some embodiments, the presently disclosed subject matter provides a respirable aerosol or nasal spray comprising the supramolecular filament described herein.

In some embodiments, the presently disclosed subject matter provides a method for treating or preventing a viral infection, the method comprising administering to a subject an amount of the supramolecular filament described herein effective to inhibit viral entry into host cells. In some embodiments, the method treats or prevents a lung injury or respiratory illness. In particular embodiments, the respiratory illness comprises coronavirus disease of 2019 (COVID-19). In some embodiments, the viral infection comprises a coronavirus infection. In some embodiments, the coronavirus infection comprises a severe acute respiratory syndrome coronavirus (SARS-CoV) or SARS-CoV-2 infection. In some embodiments, the coronavirus comprises coronavirus disease of 2019 (COVID-19). In some embodiments, the supramolecular filament is administered in a respirable aerosol or a nasal spray.

In some embodiments, the supramolecular filament described herein is delivered directly to the subject's nasal cavity or lungs. In some embodiments, the supramolecular filament is deposited within a mucus layer (through charge interactions) of the subject's nasal cavity or lungs. In some embodiments, the supramolecular filament described herein is presented over a lining of an epithelia of the subject's nasal cavity or lungs. In some embodiments, the supramolecular filament binds to the coronavirus. In some embodiments, the coronavirus bound to the supramolecular filament is cleared or degraded by one or more clearance mechanisms. In some embodiments, the one or more clearance mechanisms is associated with a macrophage or a mucociliary escalator.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

For intranasal delivery, in addition to the active ingredients, pharmaceutical compositions may contain suitable pharmaceutically acceptable carriers comprising excipients and auxiliaries which facilitate processing of the active compounds into preparations which can be used pharmaceutically. The agents of the disclosure may be formulated by methods known to those of skill in the art, and may include, for example, but not limited to, examples of solubilizing, diluting, or dispersing substances, such as saline, preservatives, such as benzyl alcohol, absorption promoters, and fluorocarbons. Optimized formulations for intranasal delivery may include addition of permeability enhancers (mucoadhesives, nanoparticles, and the like) as well as combined use with an intranasal drug delivery device (for example, one that provides controlled particle dispersion with particles aerosolized to target the upper nasal cavity).

In particular, polymer-based nanoparticles, including chitosan, maltodextrin, polyethylene glycol (PEG), polylactic acid (PLA), polylactic-co-glycolic acid (PLGA), and PAMAM dendrimer; gels, including poloxamer; and lipid-based formulations, including glycerol monocaprate (Capmul™), mixtures of mono-, di-, and triglycerides and mono- and di-fatty esters of PEG (Labrafil™), palmitate, glycerol monostearate, and phospholipids can be used to administer the presently disclosed supramolecular filaments intranasally.

The presently disclosed supramolecular filaments also can be administered intranasally via mucoadhesive agents. Mucoadhesion is commonly defined as the adhesion between two materials, at least one of which is a mucosal surface. More particularly, mucoadhesion is the interaction between a mucin surface and a synthetic or natural polymer. Mucoadhesive dosage forms can be designed to enable prolonged retention at the site of application, providing a controlled rate of drug release for improved therapeutic outcome. Application of dosage forms to mucosal surfaces may be of benefit to drug molecules not amenable to the oral route, such as those that undergo acid degradation or extensive first-pass metabolism. Mucoadhesive materials suitable for use with nasal administration of the presently disclosed supramolecular filaments include, but are not limited to, soluble cellulose derivatives, such as hydroxypropyl methylcellulose (HPMC), hydroxypropyl cellulose (HPC), methylcellulose (MC), and carboxymethyl cellulose (CMC), and insoluble cellulose derivatives, such as ethylcellulose and microcrystalline cellulose (MCC), starch (e.g., Amioca®), polyacrylates, such as poly(acrylic acid) (e.g., Carbopol® 974P), functionalized mucoadhesive polymers, such as polycarbophil, hyaluronan, and amberlite resin, and chitosan (2-amino-2-deoxy-(1→4)-β-d-glucopyranan) formulations and derivatives thereof.

In some embodiments, the formulation also includes a permeability enhancer. As used herein, the term “permeability enhancer” refers to a substance that facilitates the delivery of a drug across mucosal tissue. The term encompasses chemical enhancers that, when applied to the mucosal tissue, render the tissue more permeable to the drug.

Permeability enhancers include, but are not limited to, dimethyl sulfoxide (DMSO), hydrogen peroxide (H₂O₂), propylene glycol, oleic acid, cetyl alcohol, benzalkonium chloride, sodium lauryl sulphate, isopropyl myristate, Tween 80, dimethyl formamide, dimethyl acetamide, sodium lauroylsarcosinate, sorbitan monolaurate, methylsulfonylmethane, Azone, terpenes, phosphatidylcholine dependent phospholipase C, triacyl glycerol hydrolase, acid phosphatase, phospholipase A2, concentrated saline solutions (e.g., PBS and NaCl), polysorbate 80, polysorbate 20, sodium dodecanoate (C12), sodium caprate (CIO) and/or sodium palmitate (CI 6), tert-butyl cyclohexanol (TBCH), and alpha-terpinol.

In some embodiments, the intranasal administration is accomplished via a ViaNase™ device (Kurve Technology, Inc.) or similar devices known in the at.

In addition to the active ingredients, these pharmaceutical compositions may contain suitable pharmaceutically acceptable carriers comprising excipients and auxiliaries which facilitate processing of the active compounds into preparations which can be used pharmaceutically.

Pharmaceutical preparations can be obtained by combining the active compounds with solid excipients, optionally grinding a resulting mixture, and processing the mixture of granules, after adding suitable auxiliaries, if desired. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Supramolecular Filaments for Concurrent Ace2 Docking and Enzymatic Activity Silencing Enable SARS Coronavirus Capture and Infection Prevention

### 1.1 Overview

The design of supramolecular filaments, termed fACE2, that can both silence ACE2's enzymatic activities and immobilize ACE2 to their surface through enzyme-substrate complexation is described herein. This docking strategy enables ACE2 to be effectively delivered in inhalable aerosols and improves its structural stability and functional preservation. Moreover, fACE2 exhibits enhanced and extended inhibition of viral entry compared to ACE2 alone while mitigating lung injury in vivo. We believe this system affords a generic means for protein delivery and has high translational potential for the prevention of current and future SARS coronavirus infections.

### 1.2 Background

Numerous infectious diseases are contracted primarily via the deposition of bacteria and/or viruses into the respiratory tract, including tuberculosis, influenza, and recently COVID-19. The global COVID-19 pandemic, caused by the novel severe acute respiratory syndrome coronavirus 2 (SARS-CoV-2; also known as 2019-nCoV), has progressed into a grievous public health crisis with over 460 million confirmed cases of the disease and 6 million deaths worldwide as of Mar. 15, 2022.^{1,2} Therefore, it continues to be of paramount importance to rapidly develop effective vaccine or therapeutic strategies to address the ongoing COVID-19 pandemic and potential future epidemics.

While the FDA has provided full approval and emergency use authorization has been provided for some vaccine formulations and disease treatments,^{4,5} the virus continues to rapidly spread and subsequently mutate, leading to the emergence of Variants of Concern (VOCs) of SARS-CoV-2; exactly how these prophylactics and therapeutics will handle these and future mutations alongside new viruses is an evolving investigation.⁶⁻⁹ While SARS coronaviruses may mutate, these viruses predominantly function by their S-protein binding to cognate receptor angiotensin-converting enzyme 2 (ACE2), which is the first step for viral entry, replication, and transmission; therefore, ACE2 represents a logical druggable target for combating current and future coronaviruses.¹⁰⁻¹³ ACE2 exists in both membrane-bound and soluble forms, where both share the same enzymatic and viral binding functionalities, but only membrane-bound ACE2 is believed to facilitates viral entry and consequent infectivity.^{14, 15} Therefore, soluble ACE2 can serve as a decoy receptor by binding to the S-protein on the virus surface and thereby block the mechanism of viral entry into host cells, making soluble ACE2 an attractive candidate for preventing coronavirus infection.¹⁶⁻¹⁸

The clinical translation of soluble ACE2 remains challenging in the context of SARS-related coronavirus infections, as the enzyme is unstable and can quickly degrade, especially in an inflammatory setting.¹⁹ An additional hurdle exists in the delivery of therapeutic ACE2 to its target site (in the case of COVID-19, the airway and the lungs), where efficacy suffers from its short half-life and a lack of active transport mechanisms from the circulation into the epithelial lining fluid of the lungs if delivered by intravenous injection.²⁰⁻²² Recent advances in nanomaterials have yielded carriers of decoy ACE2 that help address these challenges, for instance the development of cell membrane-derived nanoparticles that curb SARS-CoV-2 infectivity within lung tissues.²³⁻²⁵ However, given the essential roles that ACE2, as a carboxypeptidase, plays in regulating cardiovascular function, hypertension, and innate immune systems, it remains unclear whether the delivery of enzymatically active ACE2 would lead to unknown mid- to long-term complications to the host.²⁰

### 1.3 Scope

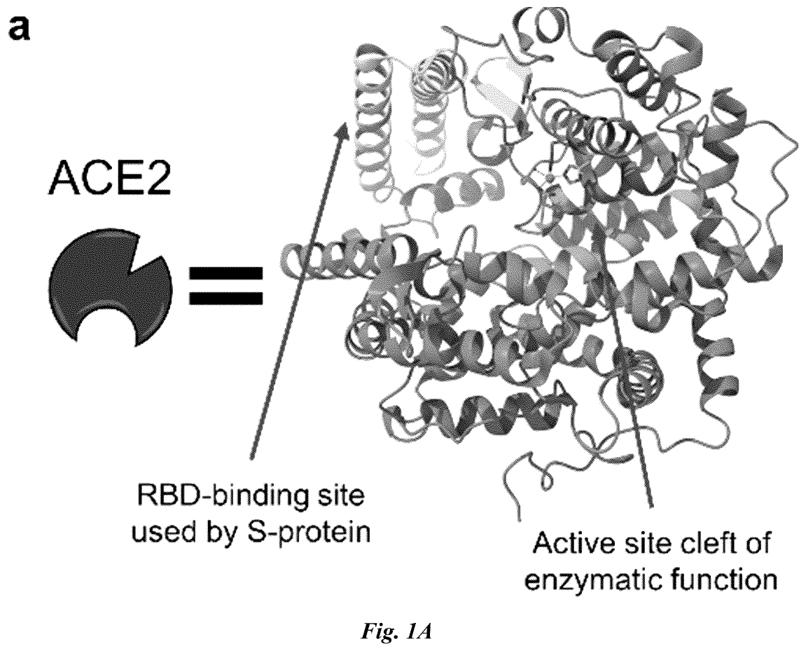

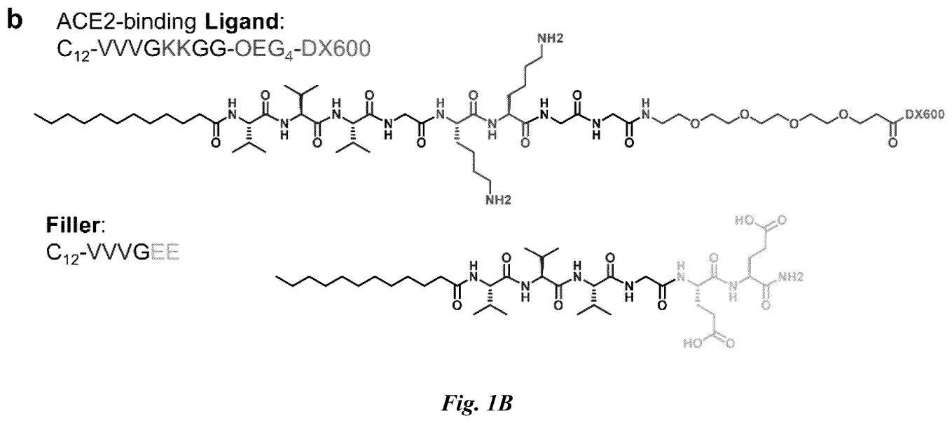

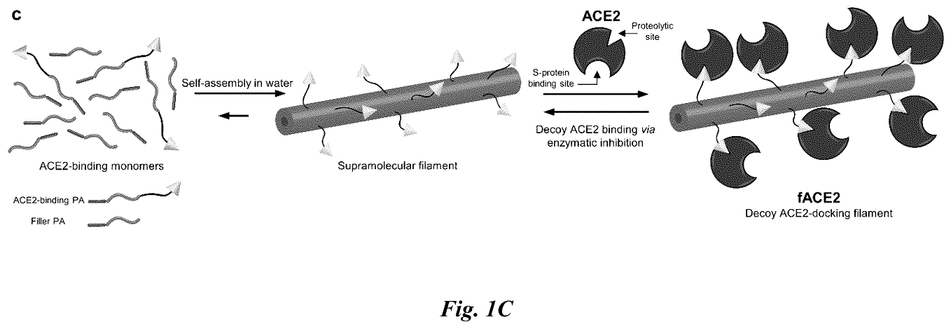

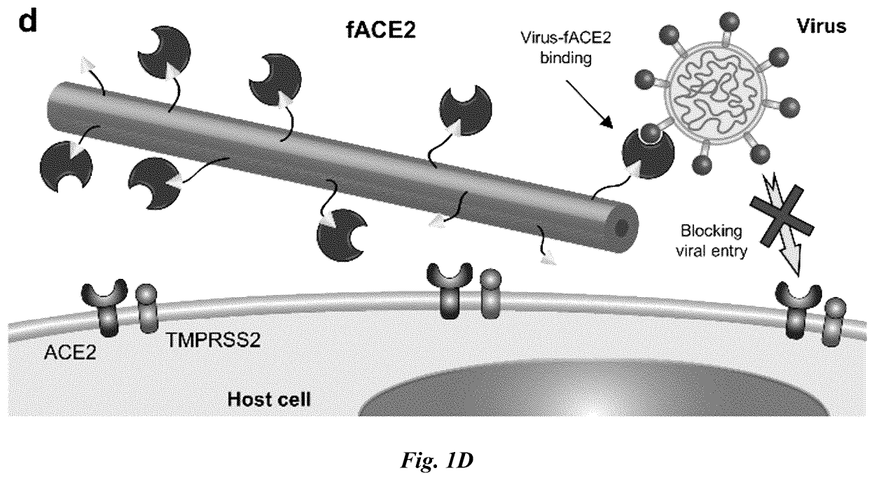

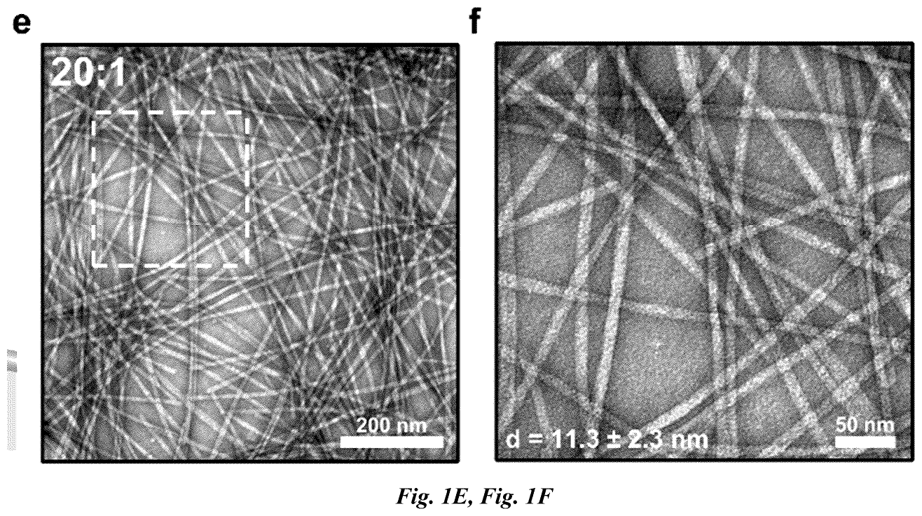

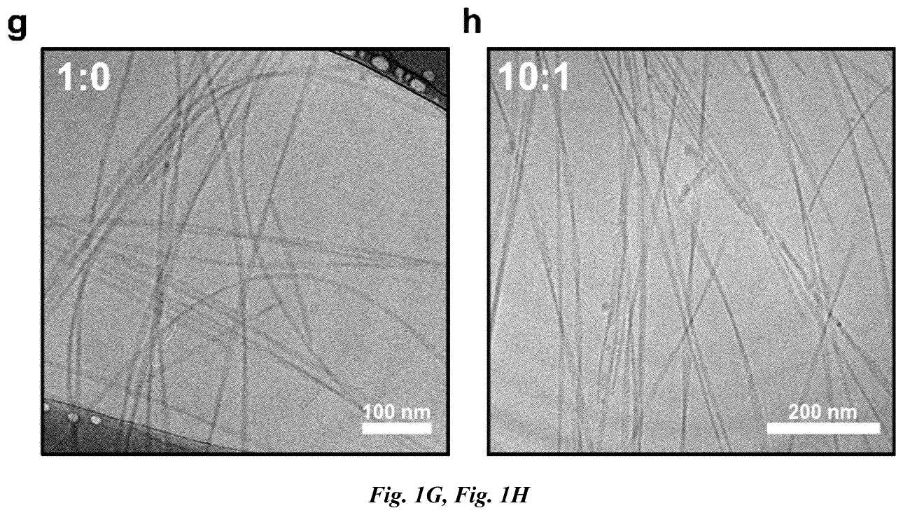

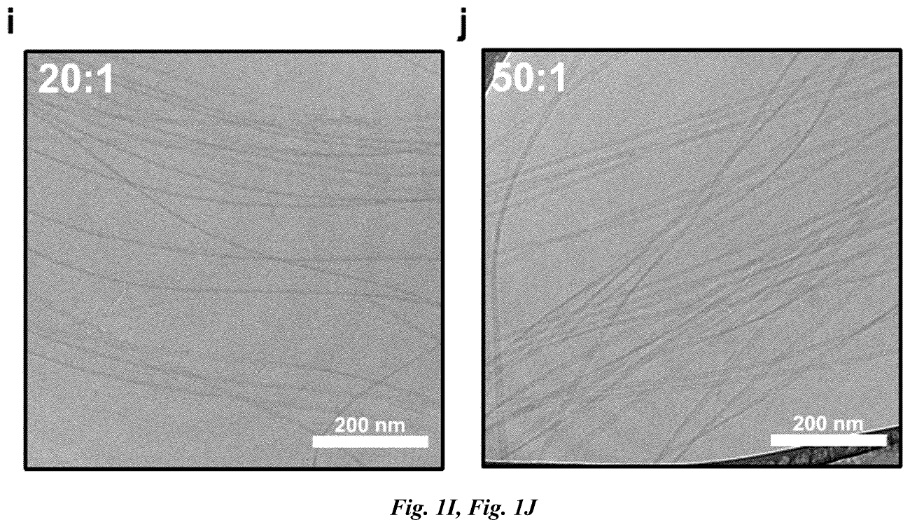

In this context, we harness the multivalent nature of ACE2, wherein the enzyme's proteolytic activity and viral receptor properties are independent and non-interfering (sites highlighted within ACE2 structure in FIG. 1A),²⁰ to both silence the ACE's enzymatic functions and display the decoy receptors on the surface of peptide-based supramolecular filamentous nanostructures via enzyme-substrate complexation. We designed peptide amphiphiles (PAs), a class of molecular building units capable of spontaneously associating in aqueous solutions to form one-dimensional supramolecular biomaterials,²⁶⁻²⁹ to present a peptide ligand/inhibitor capable of binding to the active proteolytic site of ACE2 on the filament surface. Peptide-based supramolecular materials have been designed to facilitate various supramolecular interactions to immobilize proteins on structure surfaces to enhance protein stability and delivery³⁰⁻³⁴ and could therefore be highly advantageous as carriers for decoy ACE2. In addition to being deliverable within aerosols,^{35,36} the charged surface and high aspect ratio aid in deposition and retention atop the mucus layer coating the lung epithelium while mitigating cellular internalization, thereby extending the availability of ACE2 at its target site while also mitigating potential hazardous contact of captured virus with host cells.³⁷⁻³⁹ In this work, we have developed ACE2-docking supramolecular filaments that effectively bind ACE2 through enzyme-substrate complexation to inhibit its enzymatic activities, and demonstrate the decoy function of ACE2 to effectively capture SARS coronaviruses and thereby attenuate viral infectivity in vivo.

### 1.4 Results

### 1.4.1 Design and Assembly of ACE2-Docking Supramolecular Filaments

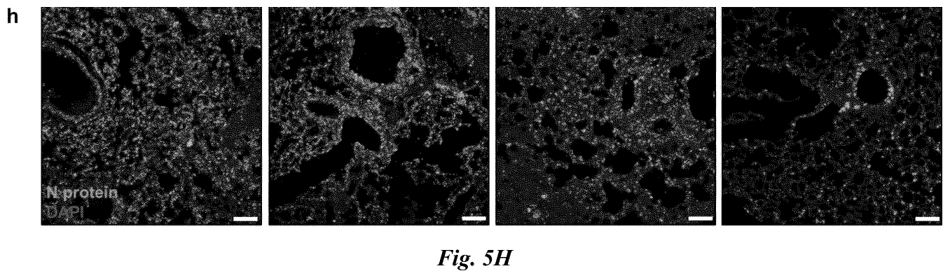

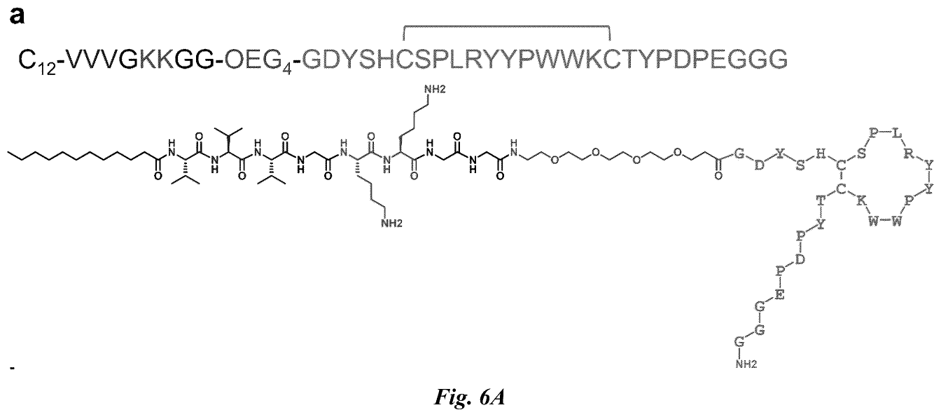

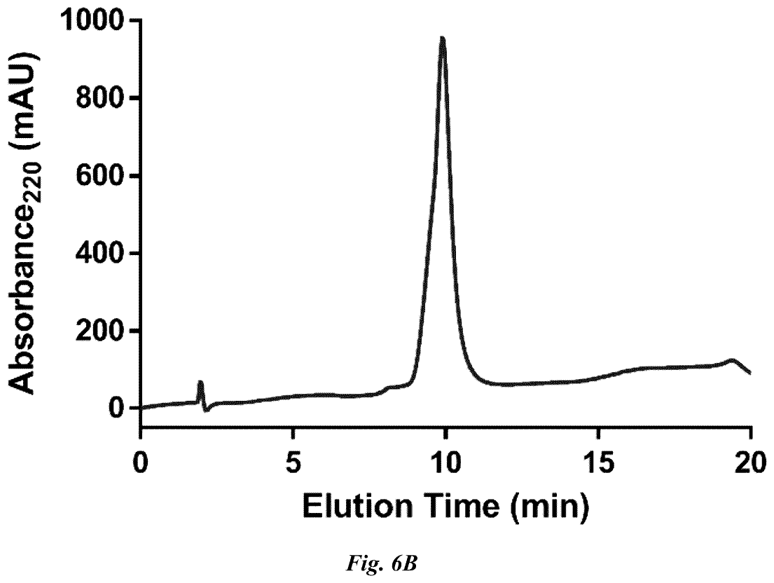

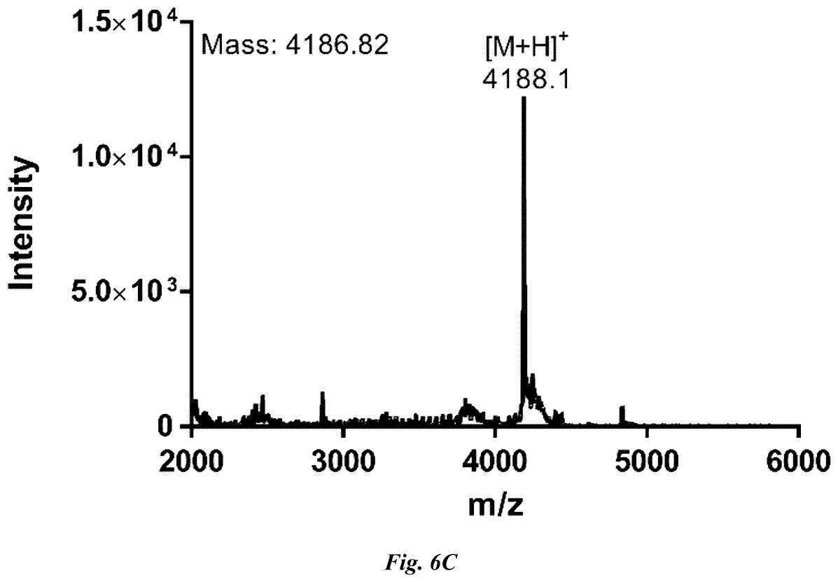

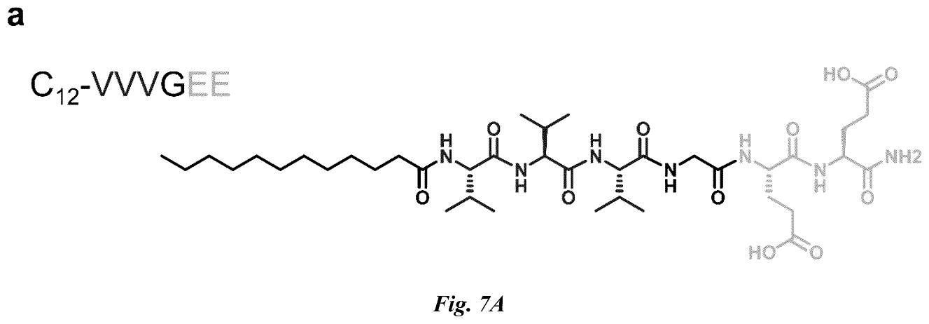

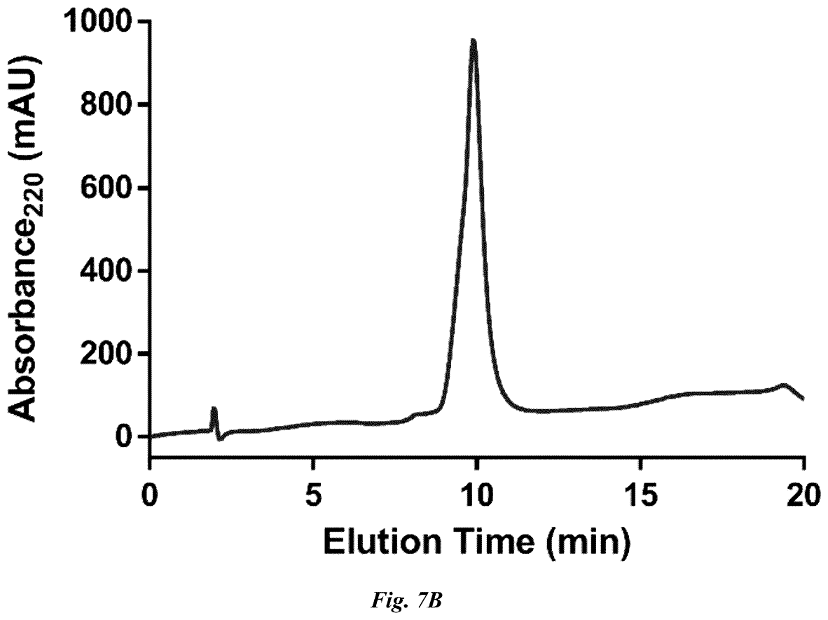

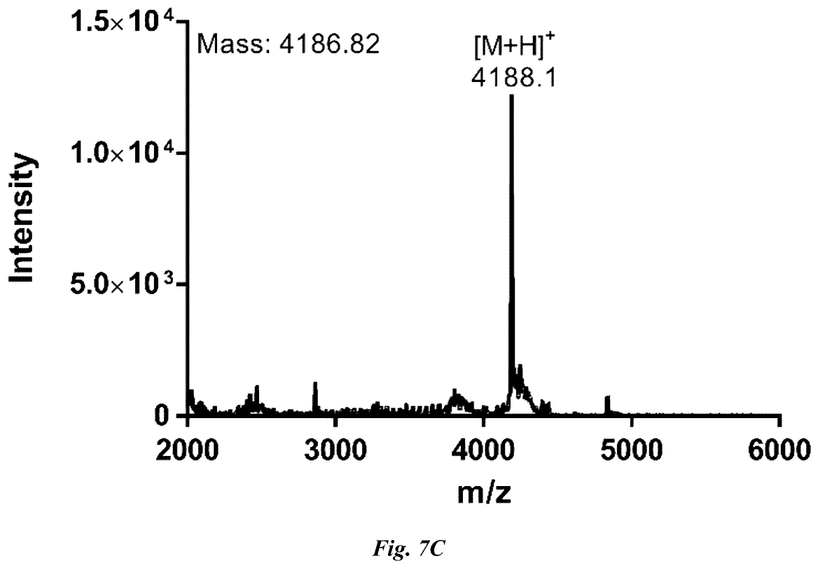

In designing our supramolecular filaments, we aimed to leverage the carboxypeptidase activity of ACE2 to allow for enzyme docking and presentation of the SARS-CoV-2 S-protein receptor-binding domain (RBD) binding site of ACE2 at the filament surface. Notably, these two sites on ACE2 are distinct and non-interfering (ACE2 structure with these sites highlighted is shown in FIG. 1A).^{11, 13, 40} Thus with this rationale, we selected a known, potent inhibitor of ACE2 enzymatic activity to incorporate into the design of an ACE2-binding peptide amphiphile (PA), termed Ligand (molecular design highlighted in FIG. 1B and FIG. 6), and paired this PA with another self-assembling constituent of the filaments, a filler PA, termed Filler (molecular design shown in FIG. 1B and FIG. 7), that serves to modulate the distribution density of the Ligand to optimize ACE2 docking and regulate surface charge.⁴¹ On the C-terminus of Ligand, we present the non-cleavable peptidic ACE2 inhibitor DX600 (Kᵢ=2.8 nM, K_{D}=10.1 nM) through which ACE2 can reversibly bind at its active site,⁴² allowing effective immobilization and release of soluble ACE2 from filament surfaces. The DX600 ligand is extended away from the filament surface with a short, flexible, hydrophilic oligoethylene glycol (OEG₄) chain and a double glycine segment (GG) as a spacer for better accessibility and to mitigate undesired interactions between ACE2 and the charged filament surface. The Ligand and Filler were molecularly crafted to have matching but oppositely charged intermolecular interaction-regulating peptide segments (VVVGKK (SEQ ID NO: 2) and VVVGEE (SEQ ID NO: 1), respectively) to facilitate the formation of a hydrogen bonding network within the filaments (VVV, SEQ ID NO:3) and to enhance supramolecular cohesion through electrostatic complexation (KK/EE), promoting co-assembly of the two components into filamentous structures.^{27,43} Both PAs contain a dodecyl chain (C₁₂ alkyl group) at their respective N-termini to enable hydrophobic collapse for self-assembly in aqueous environments. Both molecules were synthesized following standard solid-phase peptide synthesis techniques and subsequently purified and characterized with reverse-phase high-performance liquid chromatography (RP-HPLC) and MALDI-ToF mass spectroscopy, respectively (FIG. 6, FIG. 7). Together, Ligand and Filler monomers can be mixed at varying ratios in aqueous solutions to spontaneously associate and form supramolecular filaments, and subsequently, soluble ACE2 can be added to solutions of these filaments to bind to the presented ligand at the surface, yielding ACE2-docking filaments bearing decoy ACE2, called fACE2 (FIG. 1C). Solutions of fACE2 can be delivered to nasal passageways and lung tissues, where the presented decoy ACE2 can bind to SARS coronavirus spike proteins and block viral entry into host cells, curbing viral infectivity (FIG. 1D).

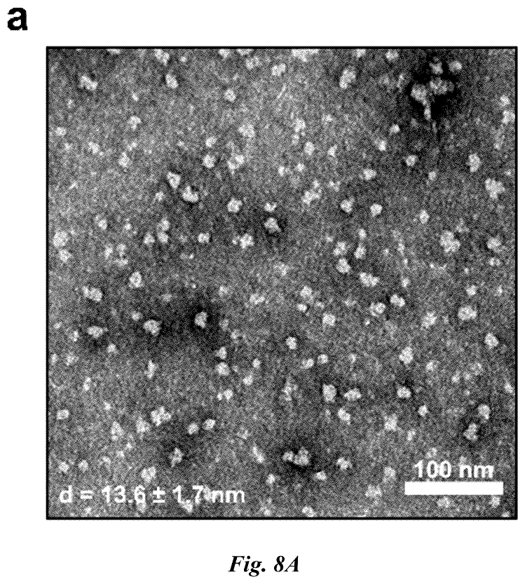

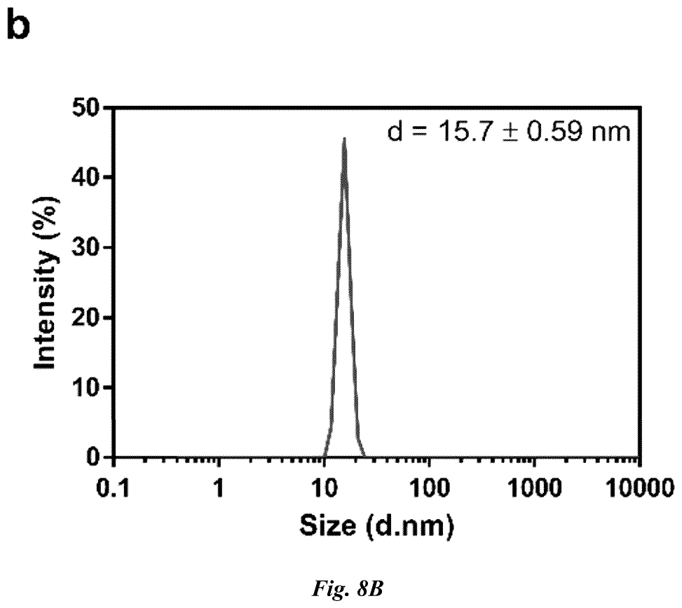

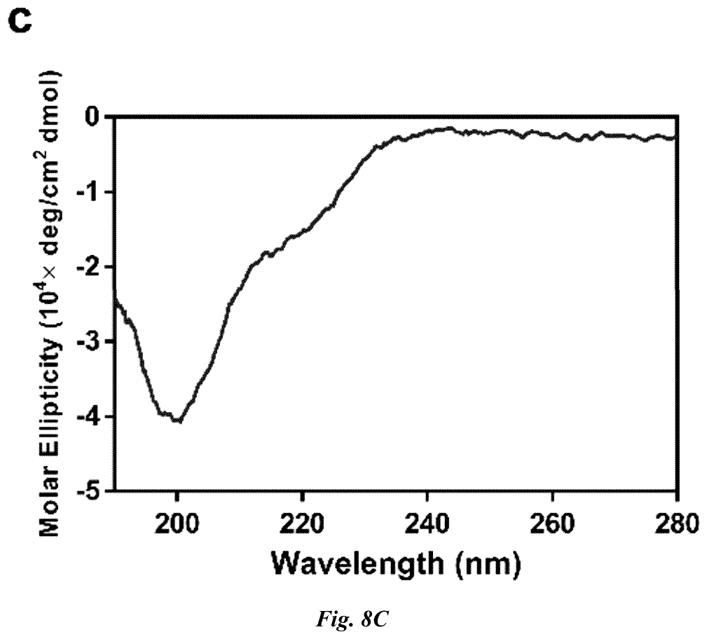

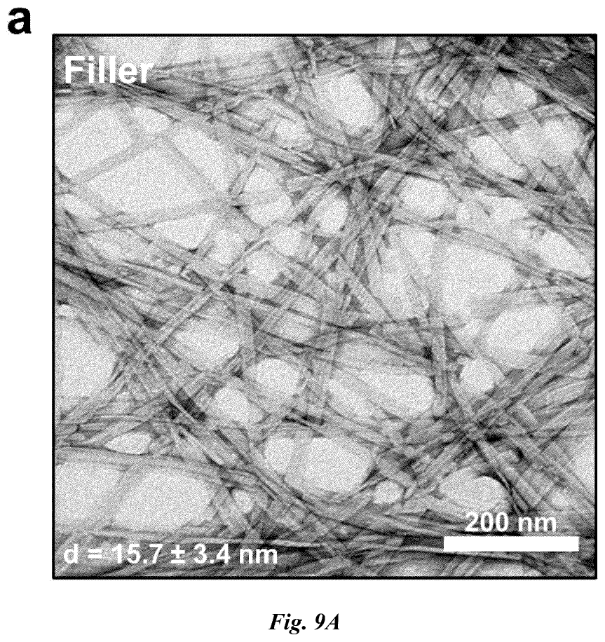

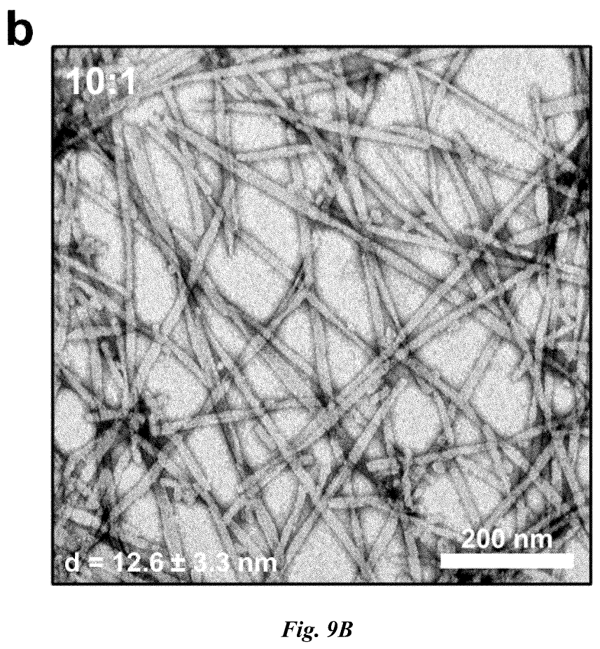

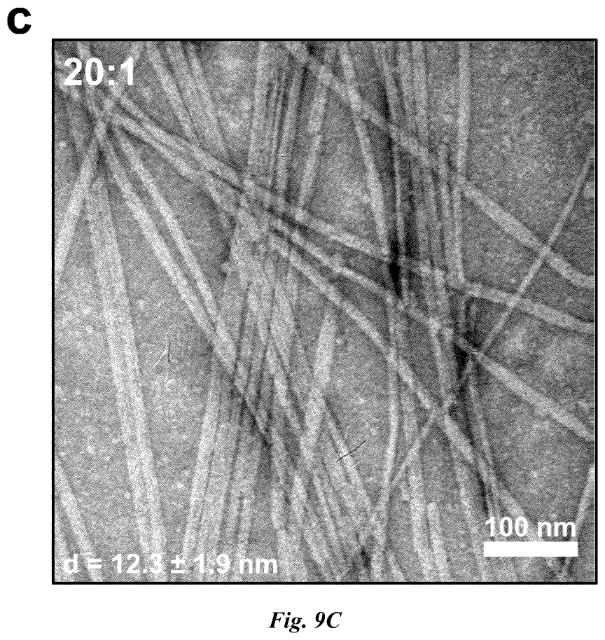

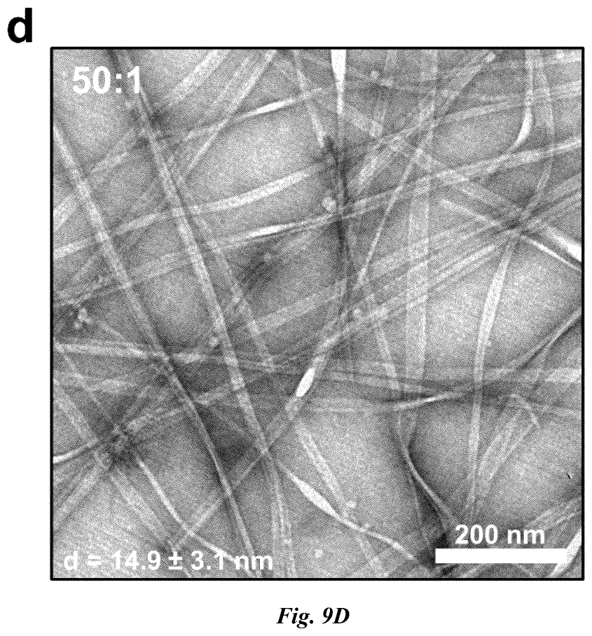

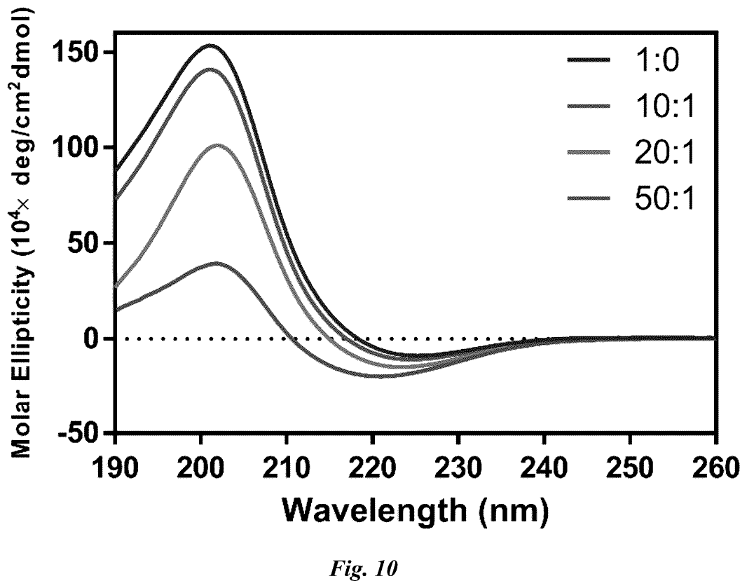

After successful synthesis and purification, the self-assembly behavior of each molecule was studied. After aging for 24 hr in water, the Ligand PA alone is observed to form spherical and other irregularly shaped aggregates using transmission electron microscopy (TEM), and this shape was corroborated by similar size measurements with dynamic light scattering (DLS) and disordered random coil circular dichroism (CD) spectra (FIG. 8), which is likely due to combination of a relatively large hydrophilic segment with steric hinderance from neighboring DX600 ligands that hinders the formation of ordered hydrogen bonds between monomers that typically yield one-dimensional structures.²⁷ The Filler PA is observed to form ribbon-like filaments over several microns in length under TEM (FIG. 9), and thus the Filler likely not only serves the purpose as a diluting agent to regulate ligand density but also key in providing dimensionality to the co-assembly of the supramolecular structure components. Indeed, when mixed together in PBS at a pH of 7.4, the Ligand and Filler PAs co-assemble into ribbon-like filaments over several microns in length with diameters measuring around ˜11.3 nm under TEM (FIG. 1E, FIG. 1F and FIG. 9), which is further corroborated by observed increasing β-sheet character of the hydrogen bonding within the filaments as Ligand content is increased (FIG. 10).

The ratio of Filler to Ligand PAs in the co-assembled structures is expected to be a key parameter in maximizing ACE2 binding, as it determines DX600 density on the filament surface,⁴¹ alongside impacting the supramolecular stability of the structures, which is critical for maintaining structural integrity during aerosol formation for inhalable delivery.³⁶ Since the assembled state represents a dynamic equilibrium between the filaments and monomers, the thermodynamic stability of each PA and their mixture was evaluated by assessing the critical micelle concentration (CMC) for each system via Nile Red assay, revealing CMC values of around 2.9, 1.3, and 0.63 μM for Filler, Ligand, and a 1:1 molar mixture, respectively (FIG. 11). By preparing filaments well above the CMC of the Ligand, we can ensure a majority of the Ligand monomers are incorporated in the supramolecular structure and thereby increase the likelihood of ACE2 docking to the filament surface. Moreover, the reduction in CMC for the mixed system corroborates the enhanced stability conferred by the additional electrostatic interactions incorporated through opposite charges in our molecular designs. We varied the molar ratios of Filler to Ligand (10:1, 20:1, and 50:1, with set 50 μM Ligand to be higher than CMC) and observed the resulting supramolecular structures under cryogenic TEM (cryo-TEM) and conventional TEM (FIG. 1G, FIG. 1H, FIG. 11, FIG. 1J, and FIG. 9). Our cryo-TEM imaging confirms the ribbon-like morphology with evidence of slight, intermittent twisting for all tested ratios in PBS, suggesting incorporation of the Ligand into filamentous structures with minimal impact on morphology.

### 1.4.2 Docking Decoy ACE2 to Supramolecular Filament Surfaces

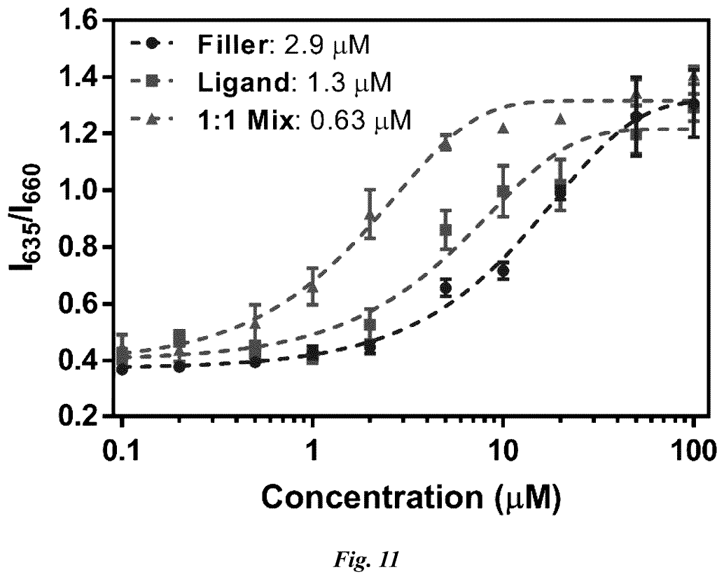

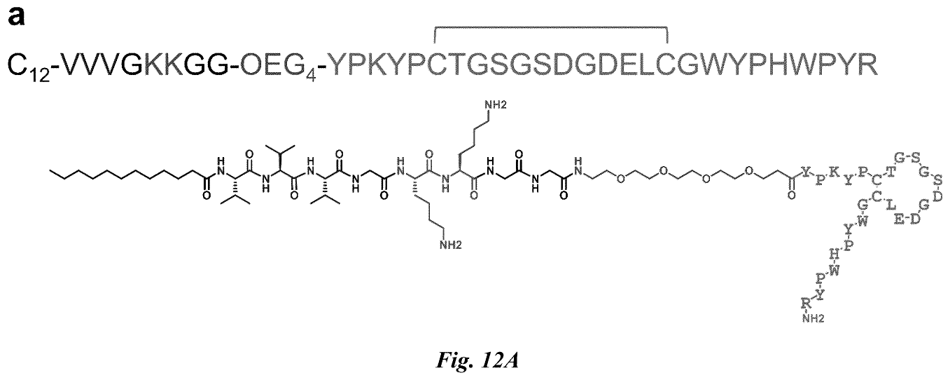

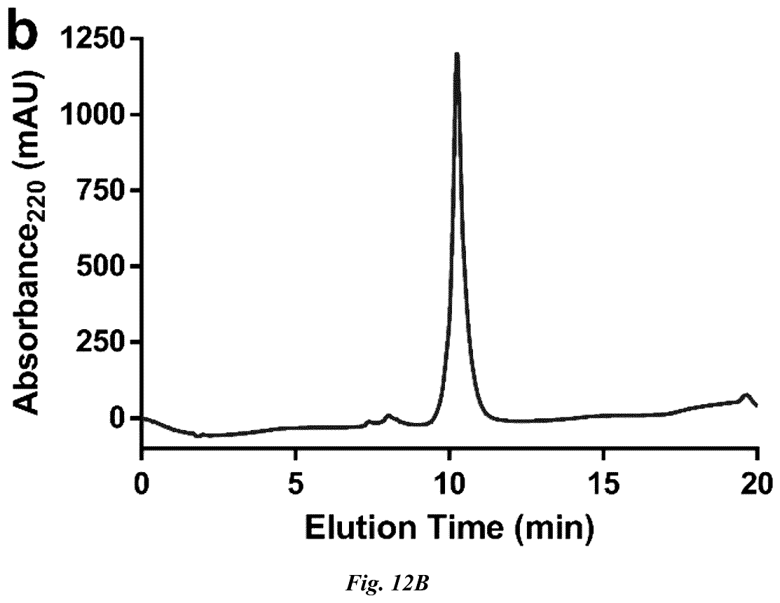

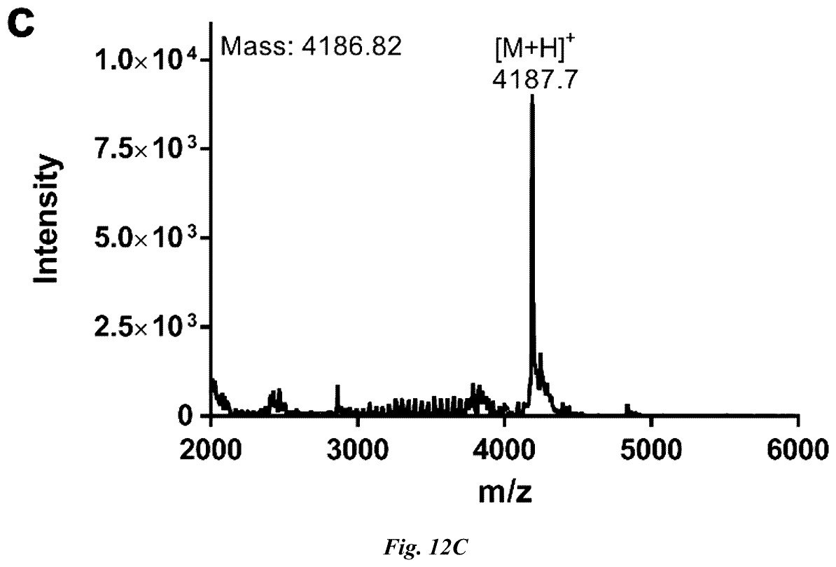

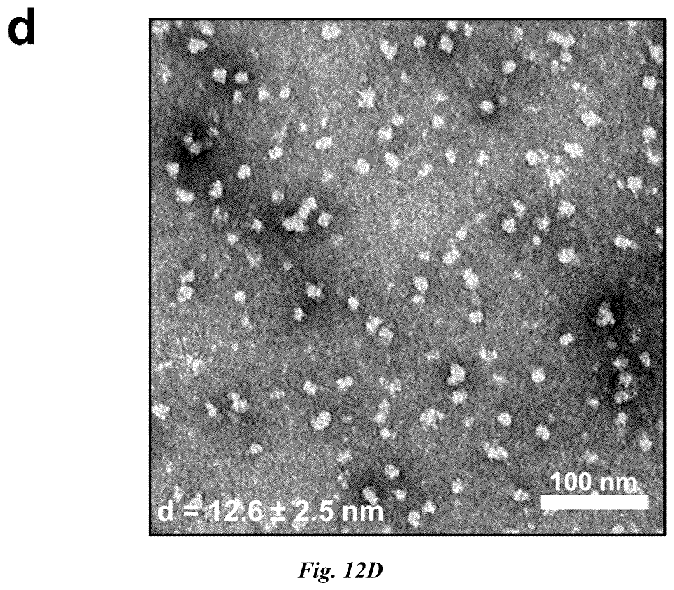

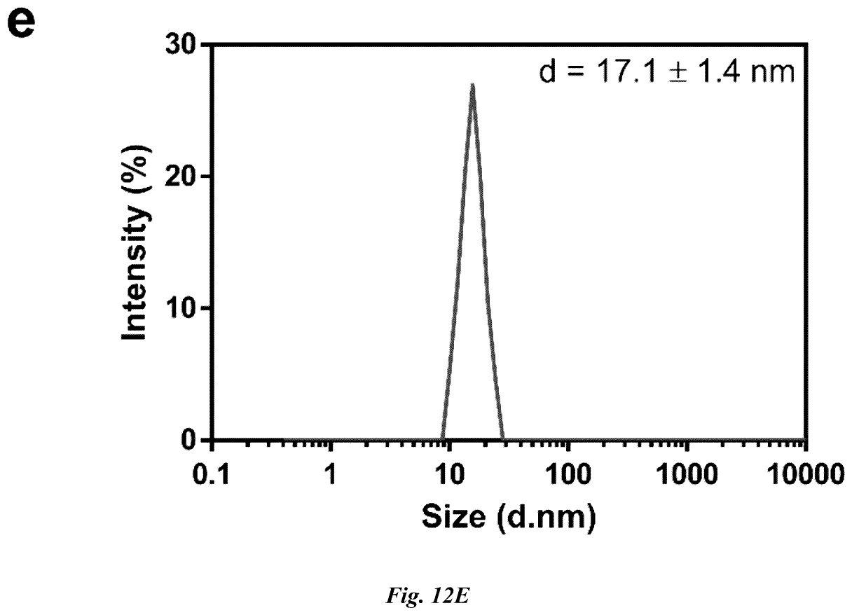

With confirmation of successful incorporation of Ligand PA into supramolecular filaments, we next aimed to assess whether ACE2 can successfully bind to the Ligand PA and dock to filament surfaces. We first aimed to assess the specificity of the binding interaction between Ligand and ACE2. We designed a scrambled analog to Ligand, termed sLigand, in which the order of the amino acids of DX600 are shuffled, to serve as a negative control (sLigand structure comprises amino acid sequence VVVGKKGGOEGOEGOEGOEGYPKYPCTGSGSDGDELCGWYPHWPYR (SEQ ID NO: 7)). Like the ACE2-specific Ligand, sLigand also forms spherical aggregates in water and PBS (FIG. 12). Binding of Ligand and sLigand to immobilized ACE2 was analyzed via bio-layer interferometry (BLI). We observed strong binding between ACE2 and Ligand compared to sLigand, which demonstrates specificity of the Ligand and ACE2 binding interaction (FIG. 2A). For high concentrations tested above the CMC value, we observe a higher background signal, indicative of nonspecific interactions occurring between the spherical aggregates and ACE2 (FIG. 2B, FIG. 2C). Due to these solubility limitations of Ligand and sLigand, a binding saturation point was not reached, and therefore an accurate binding affinity constant could not be determined. Nevertheless, these results support that a strong, specific interaction between Ligand and ACE2 does occur.

We next aimed to verify that the observed binding interaction was occurring at the proteolytic active site of ACE2 and at the surface of the supramolecular structures. Using a fluorogenic peptide substrate for ACE2 (Mca-YVADAPK(Dnp)-OH) where active ACE2 will cleave the quencher moiety from the peptide and yield detectable fluorescence signal, we assessed the impact of various components of the ACE2-docking filaments on ACE2 activity (evolved fluorescence for different conditions shown in FIG. 2D). Assuming Michaelis-Menten enzyme kinetics, we also approximated the initial velocity of the reaction (50 nM ACE2) for each condition based on the observed signal (FIG. 2E).⁴² We observed a slight impact on ACE2 activity in the presence of Filler PA alone, where the small reduction in velocity may likely be attributed to nonspecific interactions between ACE2 and the filaments and also to diffusion limitations presented by a dense filament network (1 mM Filler); however, this activity is much higher compared to the Ligand alone (50 μM), highlighting the DX600 peptide design is key for the ACE2 interaction/inhibition. Moreover, when compared to the sLigand (50 μM), the Ligand drastically reduces ACE2 initial velocity, confirming that the ACE2-Ligand interaction is occurring at the ACE2 proteolytic site. Indeed, though slightly higher, there is no appreciable difference in the initial velocity of ACE2 cleavage in the presence of Ligand in comparison to the free DX600 peptide alone. For the co-assembled system (20:1 molar ratio Filler to Ligand), we observed a higher initial velocity compared to the Ligand alone, despite equal Ligand concentrations. This could likely be due to a more confined orientation of the DX600 peptide presented on the filament surface, limiting accessibility to ACE2 to some extent, in combination with slower diffusion of ACE2 through the filament network. Nevertheless, this reduction relative to the initial velocity of free ACE2 shows around 93% inhibition of added ACE2, suggesting binding to presented inhibitor ligands on the docking filaments.

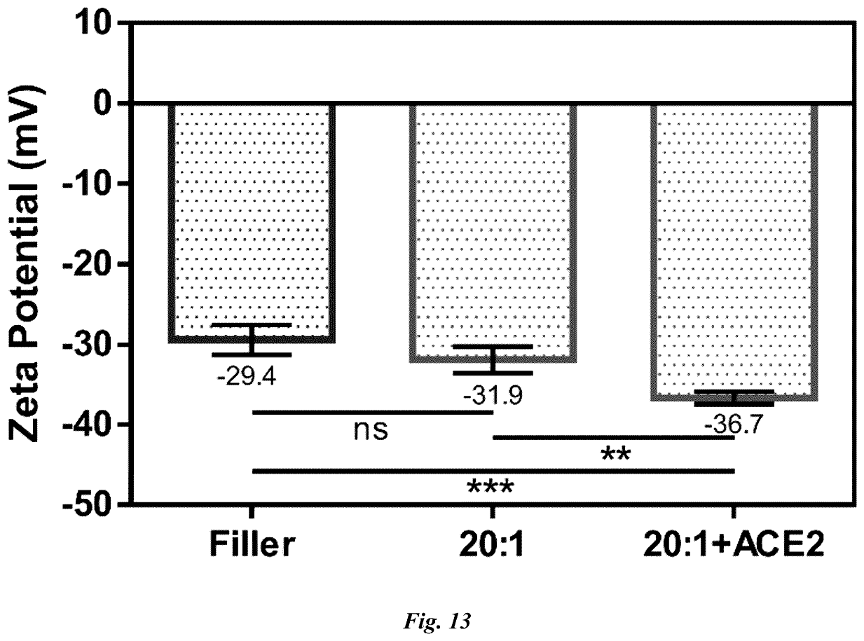

Next, we aimed to further validate that the binding interaction with ACE2 occurs at the surface of the supramolecular structures and not predominately and/or exclusively with monomers in solution. We first conducted zeta potential measurements of ACE2 alone (pI≈5.36) and in the presence of Ligand and sLigand spherical aggregates at physiological pH (7.4) in PBS (FIG. 2F), for if the two entities are not interacting, we expect the measured zeta potential to be equivalent to the intensity-averaged zeta potential of the mixture. Indeed, we measured a large drop in zeta potential for the Ligand micelles mixed with ACE2 and relatively no change with the sLigand system, suggesting ACE2 complexation at the particle surface and further emphasizing the key role of Ligand binding in facilitating this interaction as opposed to other nonspecific interactions. The lower zeta potential is expected with ACE2 binding, as the exposed spike RBD-binding site on ACE2 exhibits a negative electrostatic potential.⁴⁴ This same phenomenon is also observed with the co-assembled supramolecular system, where the zeta potential decreases after ACE2 binding, suggesting ACE2 docking at the filament surface (FIG. 13).

### 1.4.3 Optimizing ACE2 Presentation on Supramolecular Filament Surface

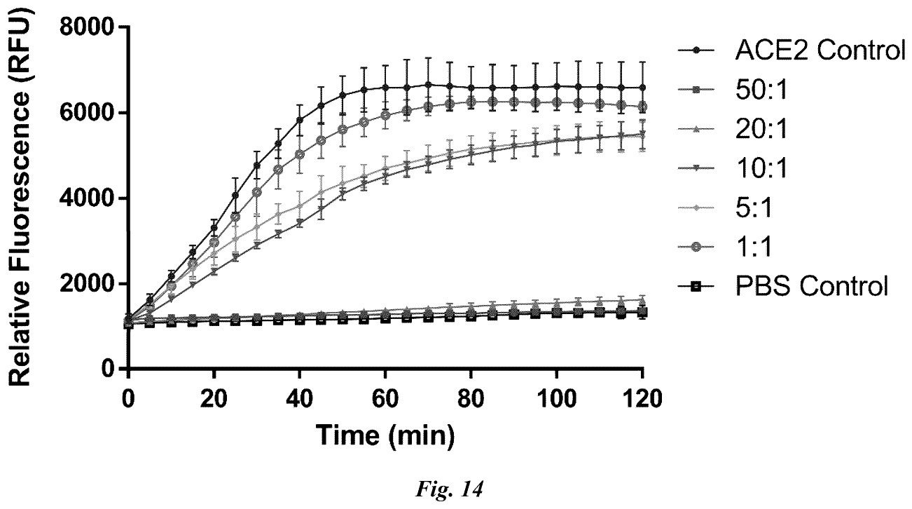

To maximize the docking efficiency of added ACE2 and optimize the presentation of ACE2 on the surface of the ACE2-docking supramolecular filaments, we investigated the effects of different co-assembly variables in facilitating ACE2 docking, such as molar ratios of the two filament components and relative ACE2 content. First, we examined the Filler:Ligand molar ratio on the ACE2 docking efficiency, which we represent as the extent of observed inhibition of ACE2 enzymatic activity for each tested group (initial velocity using fluorogenic peptide substrate assay) in comparison to free ACE2 activity at the same conditions. For fixed concentrations of Ligand and ACE2 (50 μM and 50 nM, respectively), we observe that with an increasing Filler content relative to Ligand, we achieve greater ACE2 docking (FIG. 2G, FIG. 14). This is likely reflective of enlarged spacing between neighboring ligands, which facilitates effective ACE2 binding by mitigating steric hinderance that may result from crowding of ligands and/or ACE2 (˜85 kDa). With at least a 20:1 molar ratio of Filler:Ligand, we achieve around 96% of the added ACE2 bound at the filament surface, and higher ratios from this point achieve minimal increases in ACE2 incorporation. We therefore selected the 20:1 ratio as the optimal spacing, as this also minimizes Filler demand, which in turn, decreases the total number of filaments and likelihood of physical crosslinks that can increase solution viscosity and potentially impede ACE2 diffusion.^{32, 45}

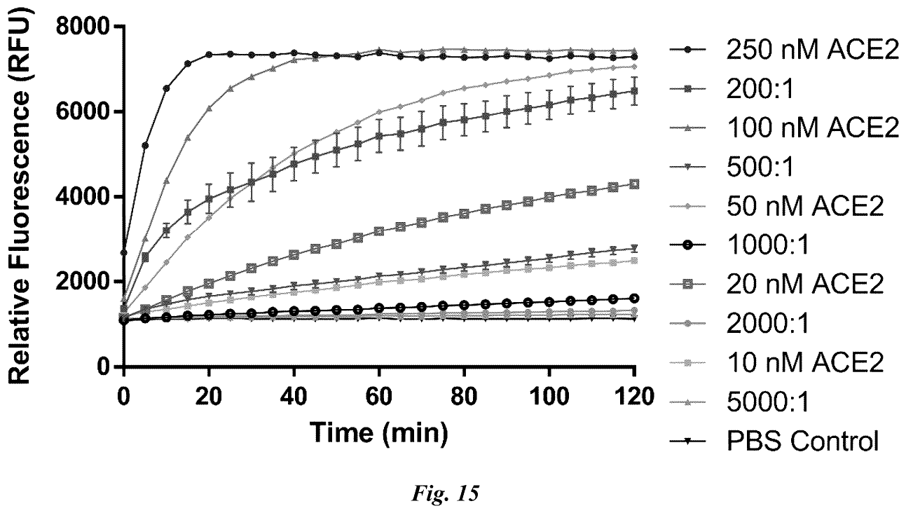

Considering the binding equilibrium that exists between our Ligand, ACE2, and the Ligand-ACE2 complex, we next investigated the effects of the molar Ligand:enzyme ratio, a key parameter in enhancing docking efficiency. Holding the Filler:Ligand ratio (20:1) and Ligand concentration (50 μM) constant, we varied the Ligand:enzyme ratio by adjusting the added ACE2 concentration (10-250 nM) and determined its impact on ACE2 activity inhibition. As expected, we find that with increasing the Ligand:enzyme ratio, the docking efficiency increases (greater observed enzymatic inhibition), as binding equilibrium is shifted toward the formation of the Ligand-ACE2 complex (FIG. 2H, FIG. 15). With a 1000:1 molar ratio of Ligand:ACE2 at our optimal ligand spacing, we successfully dock around 95% of the added ACE2 to the filament surface, where further increases show negligible changes in docking efficiency. At higher ACE2 concentrations, docking may be limited by the accessibility of ligands at the filament surface or other steric effects. Therefore, we selected the 1000:1 molar ratio of Ligand:ACE2 as optimal for future preparations of fACE2, as this ratio ensures almost all added ACE2 will bind to the filament surface.

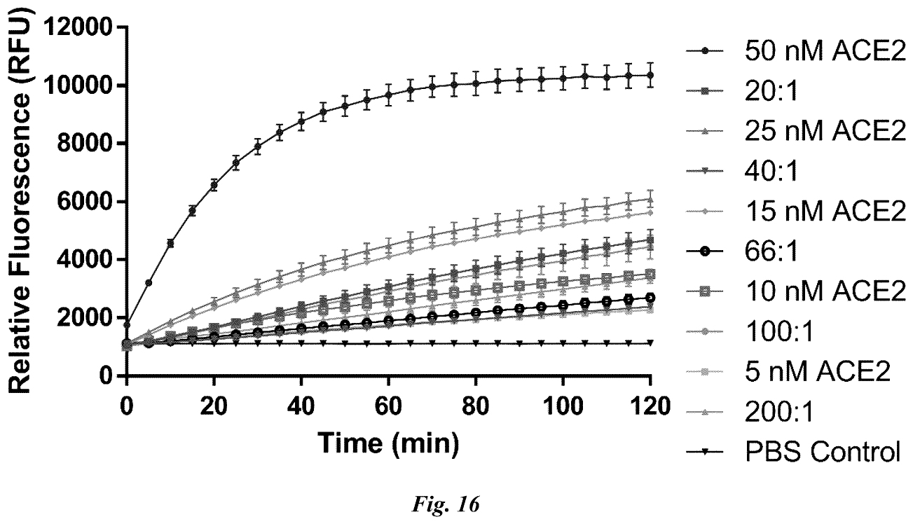

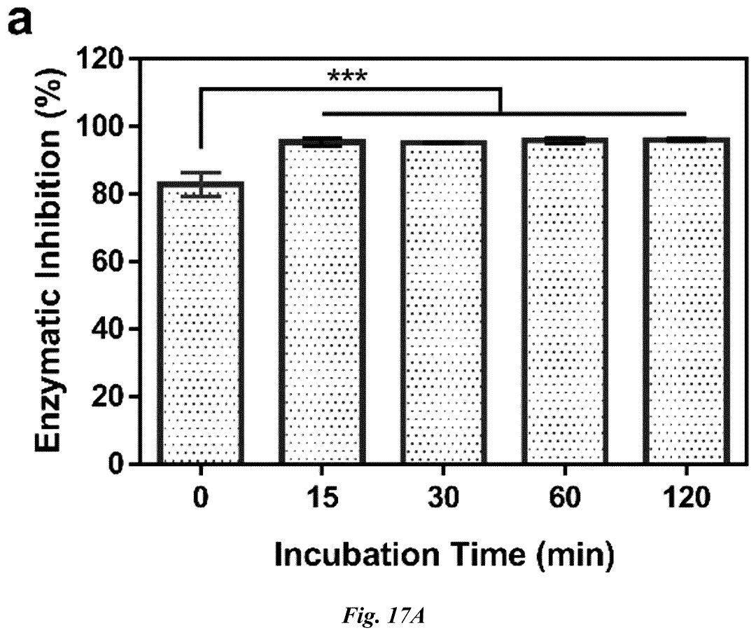

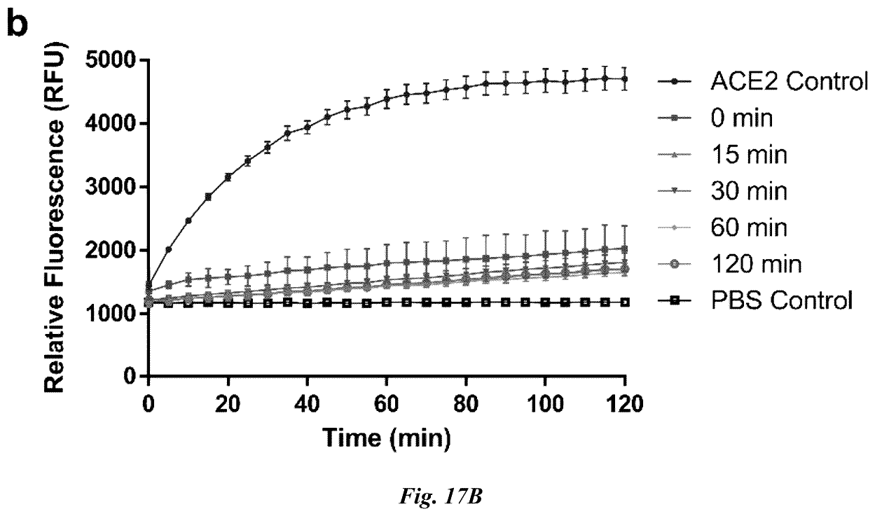

After examining the role of Filler concentration and the ratio of Ligand to ACE2, we next studied the impact of Ligand concentration in maximizing docking efficiency. While holding the Filler concentration constant (1 mM) and varying added ACE2 to maintain a 1000:1 molar ratio of Ligand:ACE2, we adjusted the Ligand content within the filaments (5-50 μM) and determined its impact on ACE2 activity (FIG. 2I, FIG. 16). We observe that increasing the ligand concentration yields greater docking of ACE2, as expected. Moreover, we see a drastic drop in ACE2 docking with the lower concentrations of Ligand tested (10 μM), suggesting the existence of a critical point in Ligand concentration, where despite being at the optimal ratio relative to ACE2, the density of Ligand is too low, and equilibrium is likely not shifted in favor of formation of the Ligand-ACE2 complex. Additionally, these results further validate that without the presentation of the enzymatic inhibitory ligand at their surface, there is negligible interaction of ACE2 with the filaments. Lastly, we determined the minimal incubation time sufficient to achieve the maximum docking efficiency of added ACE2. At the previously determined optimal conditions for ACE2 docking (20:1 Filler:Ligand and 1000:1 Ligand:ACE2), we pre-incubated filaments with ACE2 (25 nM) for a range of times (0-120 min) before assessing ACE2 activity (FIG. 17). We observed an almost instantaneous capture of ACE2 to the filament surface (0 min incubation, ˜83% inhibition), which is likely due to the strong binding affinity of the DX600 ligand for ACE2. Indeed, within 15 minutes, we achieve the maximum docking efficiency of around 95%, with longer incubation times showing negligible increases in ACE2 inhibition. This is promising with respect to the translation of the system to a clinical setting, as ACE2 will dock to filament surfaces within a few minutes, yielding fACE2 ready for administration.

1.4.4 Delivery Off ACE2 within Respirable Aerosols

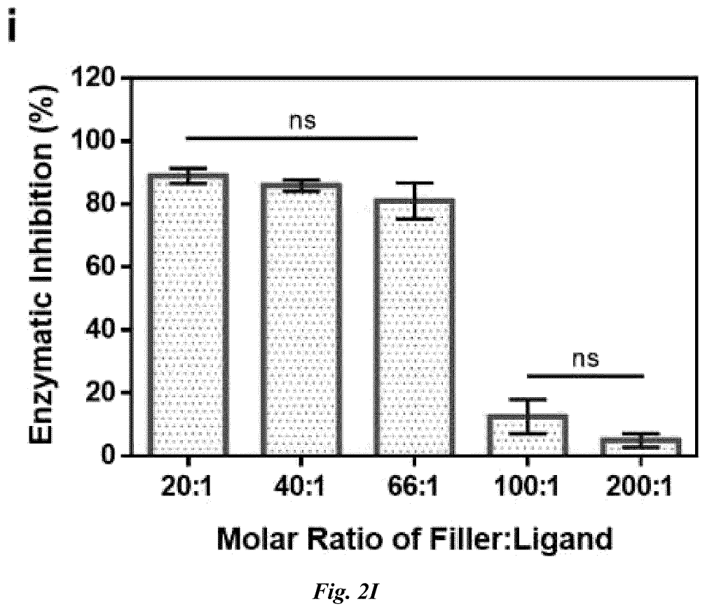

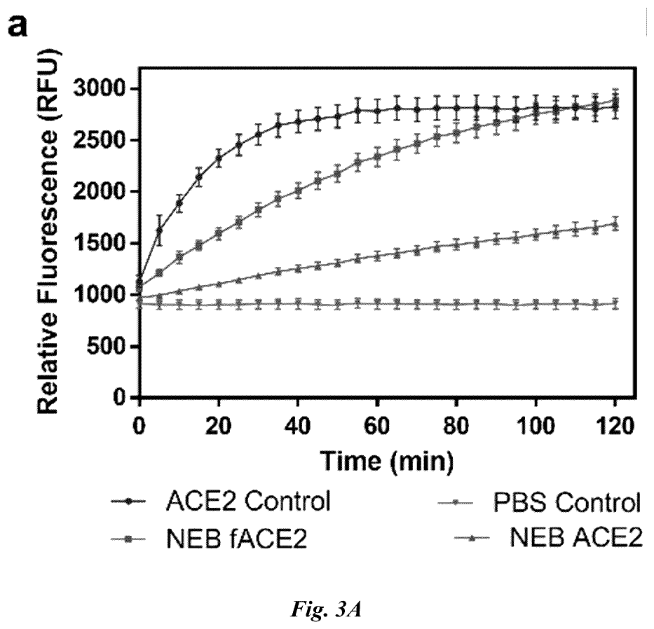

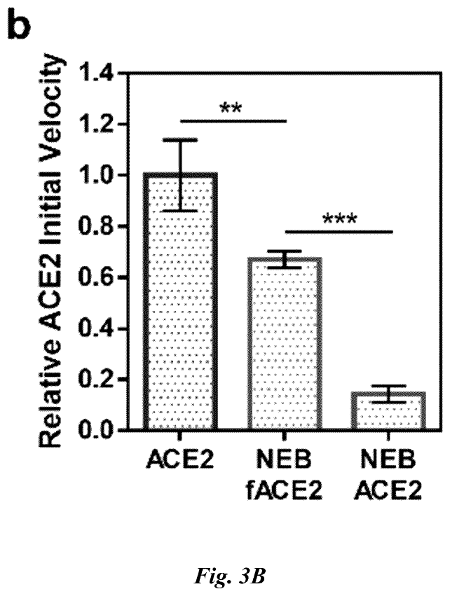

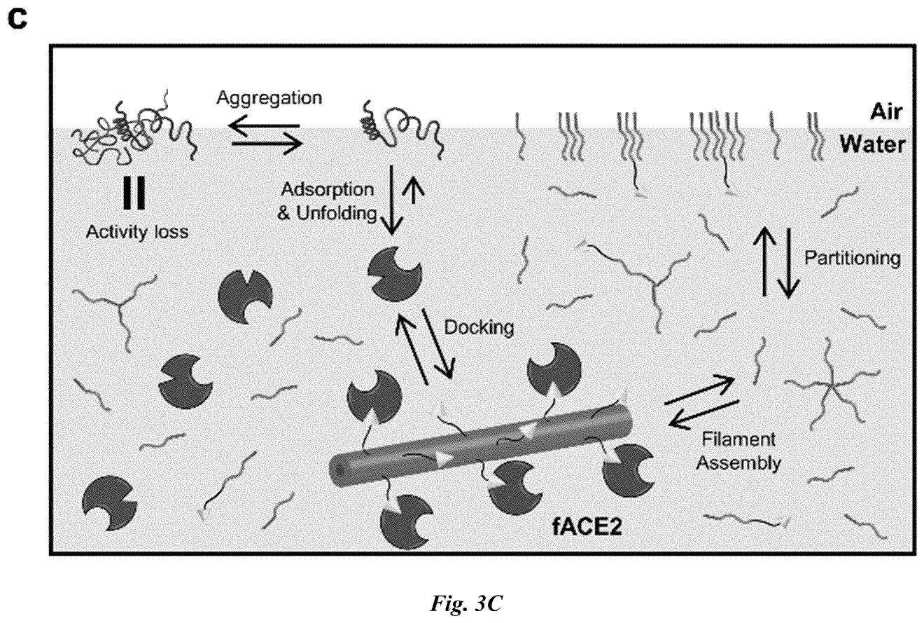

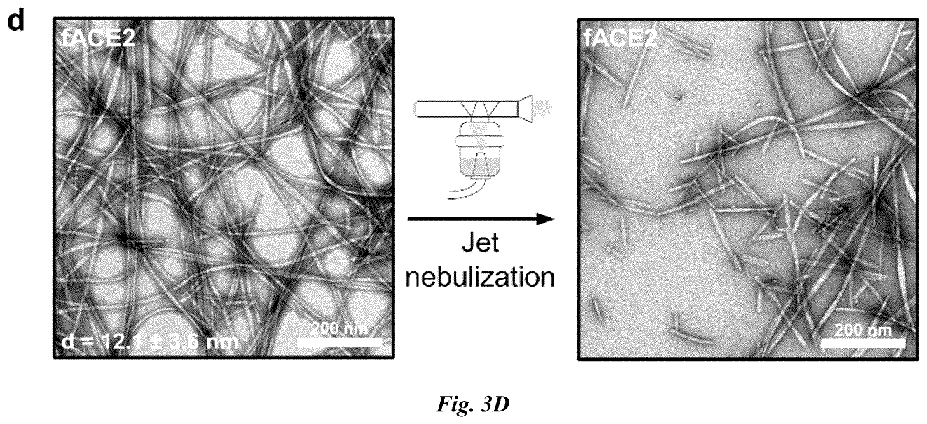

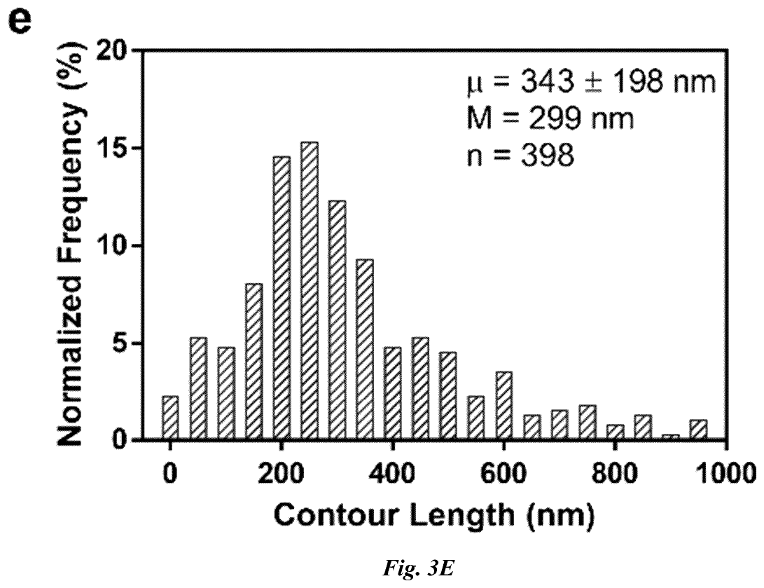

Having validated successful docking of ACE2 to the supramolecular filament surface, we next evaluated the ability of the filaments to carry ACE2 within respirable aerosols as a means of delivering ACE2 directly to lung tissues via jet nebulization. Due to the noncovalent nature of both filament assembly and binding of ACE2, we expect structural integrity and activity to be impacted by air-liquid interface (ALI) enrichment and shear stress during aerosol droplet formation.^{36, 46, 47} This is of particular importance with respect to the decoy function of ACE2, as potential ACE2 unfolding and aggregation from aerosolization may negatively influence the ability of the viral S-protein to effectively bind to decoy ACE2. We therefore investigated the stability of docked ACE2, reflected in its enzymatic activity (as unfolded and/or aggregated ACE2 will likely exhibit inhibited activity), by collecting and analyzing the emitted mist from a jet nebulizer. Strikingly, though reduced compared to pre-nebulization, we find that ACE2 delivered on filaments exhibits much higher enzymatic activity (around 67% relative initial velocity to ACE2 control) compared to free ACE2 (around 14% relative to control) after jet nebulization (FIG. 3A, FIG. 3B). These results suggest that our docking strategy not only facilitates delivery of decoy ACE2 in respirable aerosols but also provides protection against protein denaturization by harsh aerosolization forces, ensuring greater fractions of delivered ACE2 in the correct conformation. We speculate that two factors contribute to the structural preservation of ACE2 in this system (represented in FIG. 3C). First, the strong binding affinity of ACE2 to the Ligand molecule likely aids in mitigating adsorption of ACE2 to the highly hydrophobic ALI, which can result in protein unfolding and aggregation, by shifting equilibrium toward the docked state and reducing ACE2 content in the bulk solution. Second, the PA monomers of the supramolecular filaments exchange frequently between their assembled state and the ALI, where the hydrophobic influence of the ALI shifts assembly-disassembly equilibrium from the supramolecular structure to monomeric form.^{36,48} Subsequent enrichment of the ALI by PA monomers likely impedes ACE2 adsorption and potential unfolding and/or aggregation, thereby preserving its structure and activity. Therefore, using supramolecular filaments as inhalable carriers appears highly advantageous for protein delivery within respirable aerosols, particularly due to the surface activity of PAs.

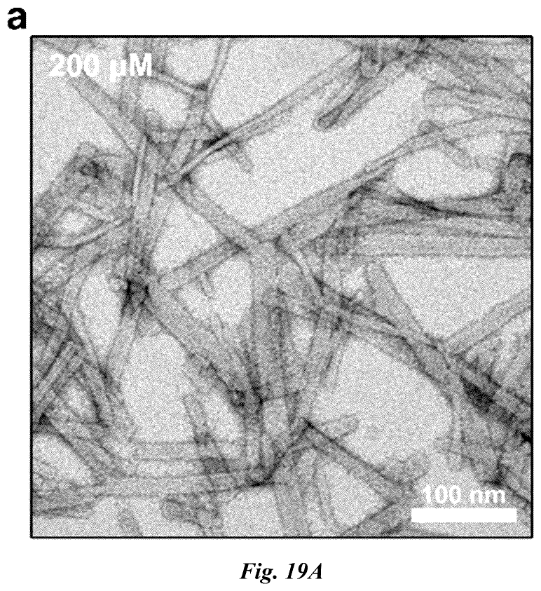

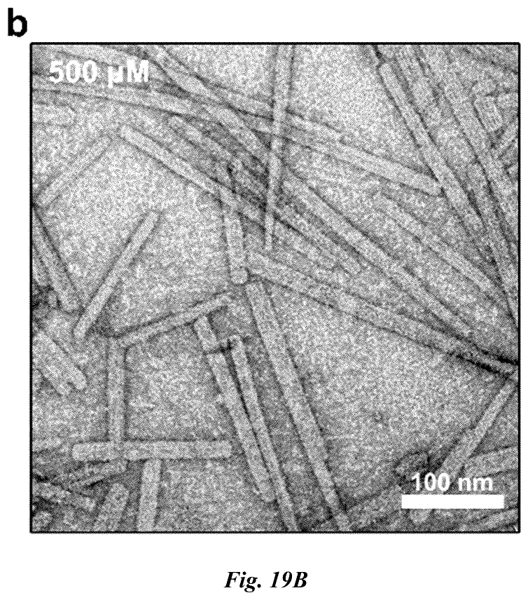

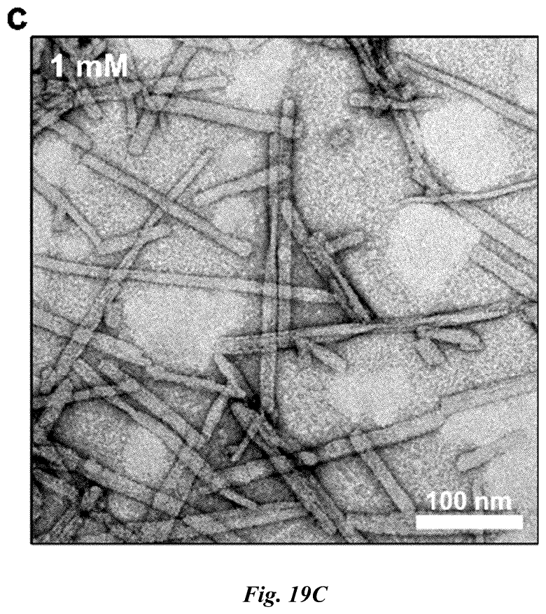

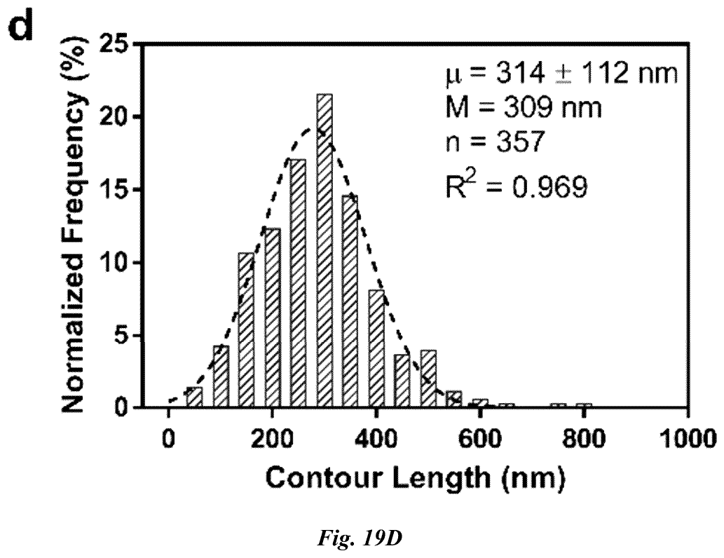

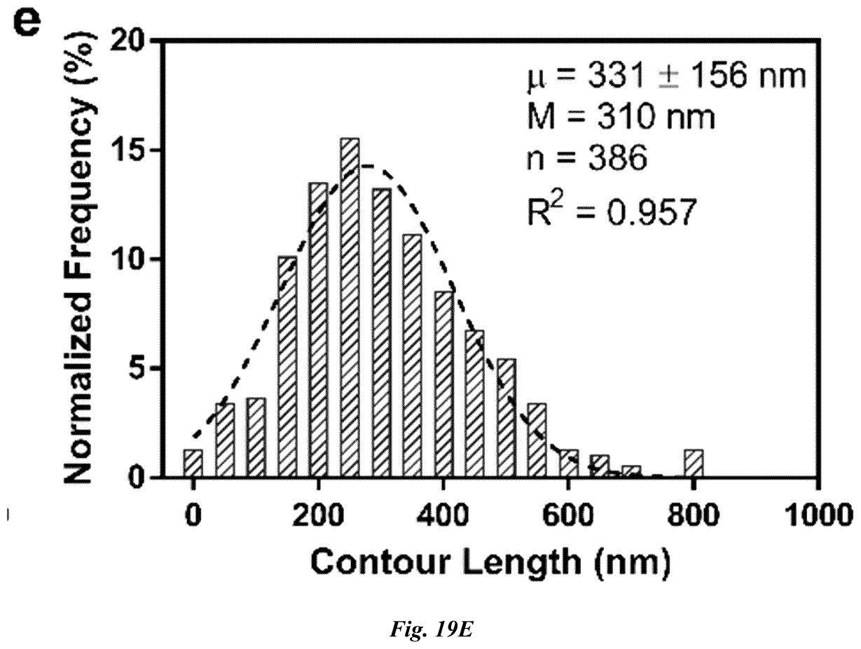

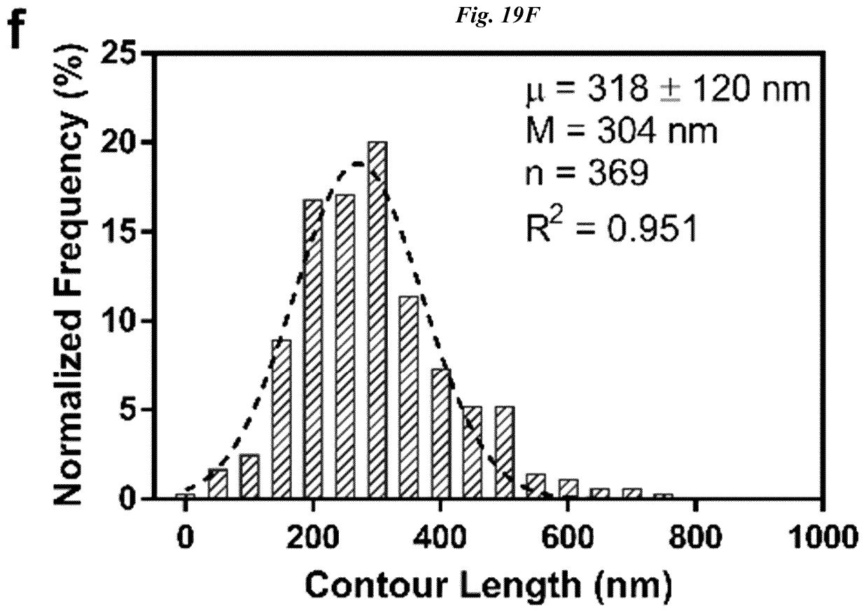

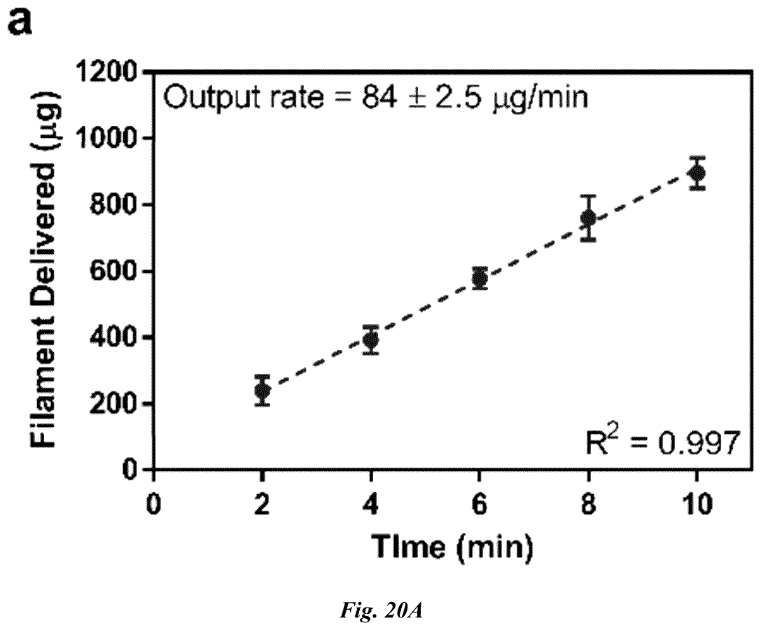

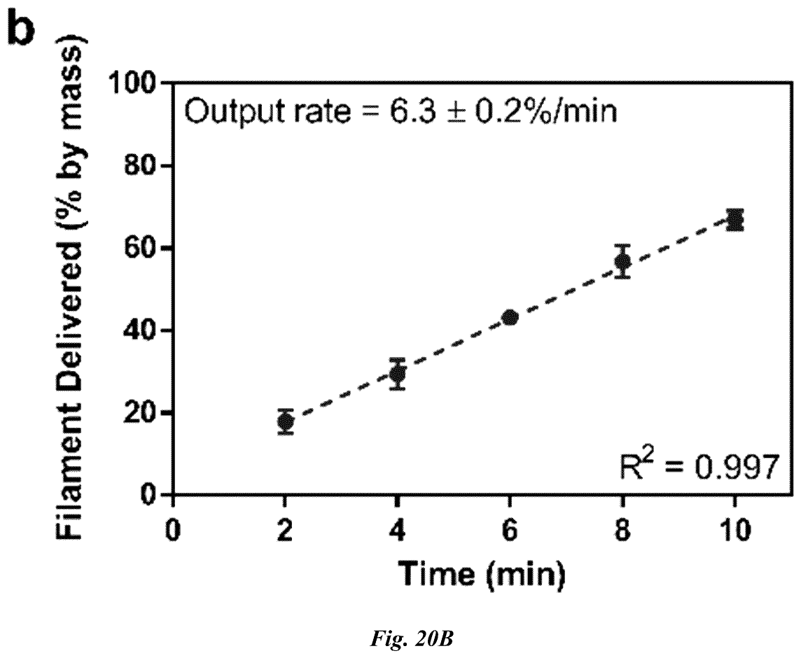

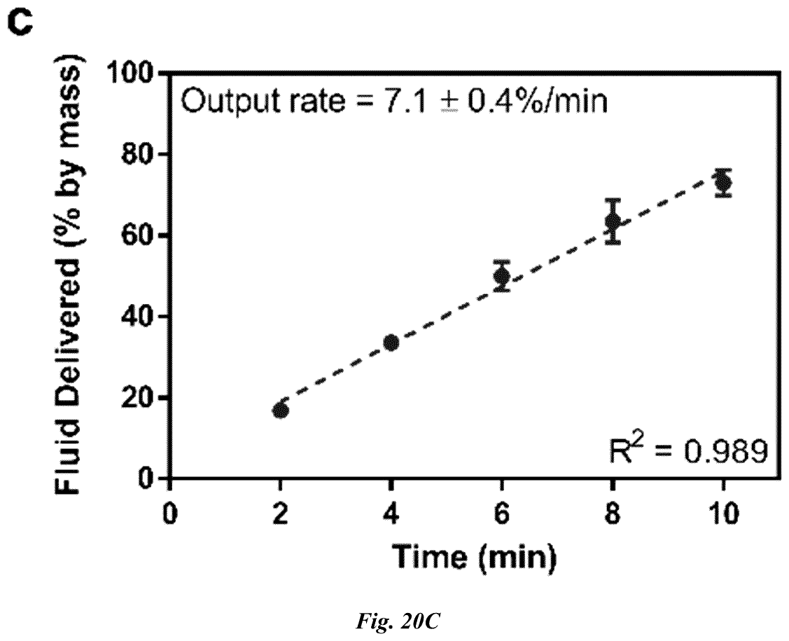

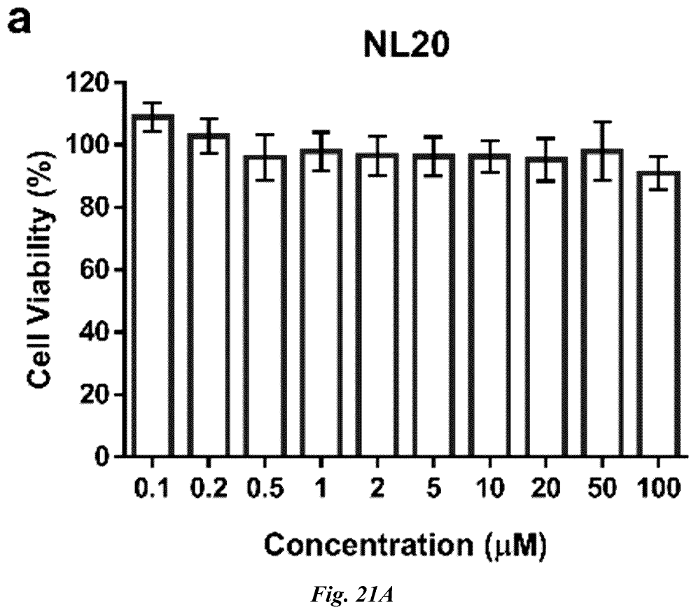

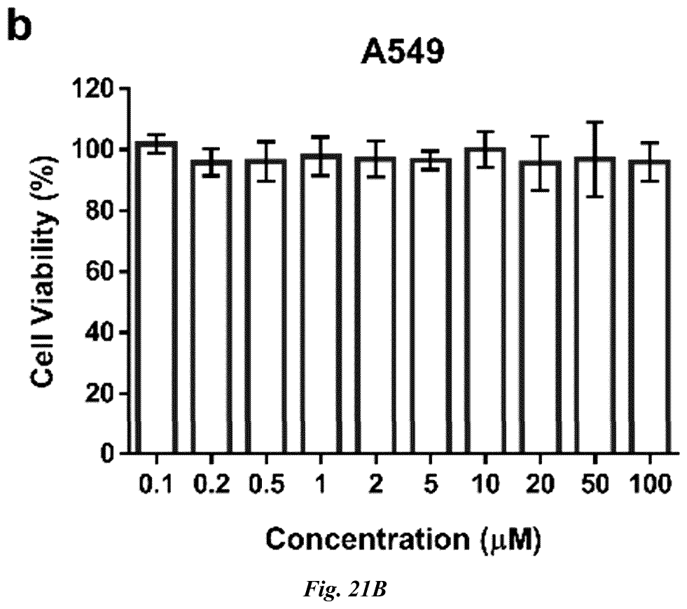

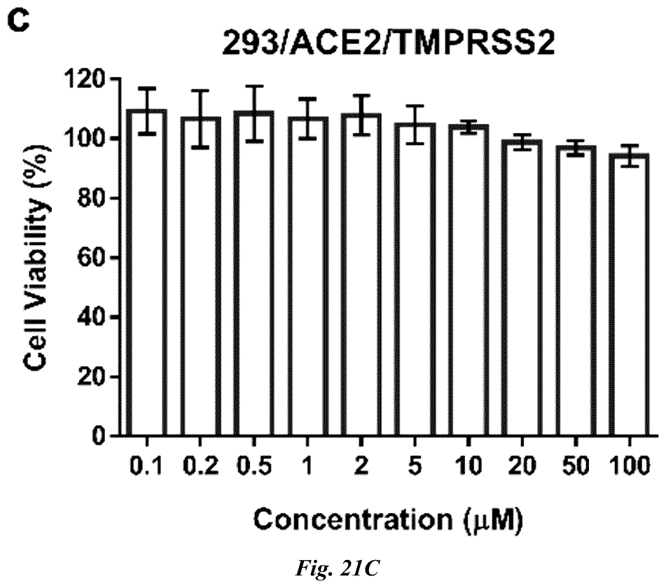

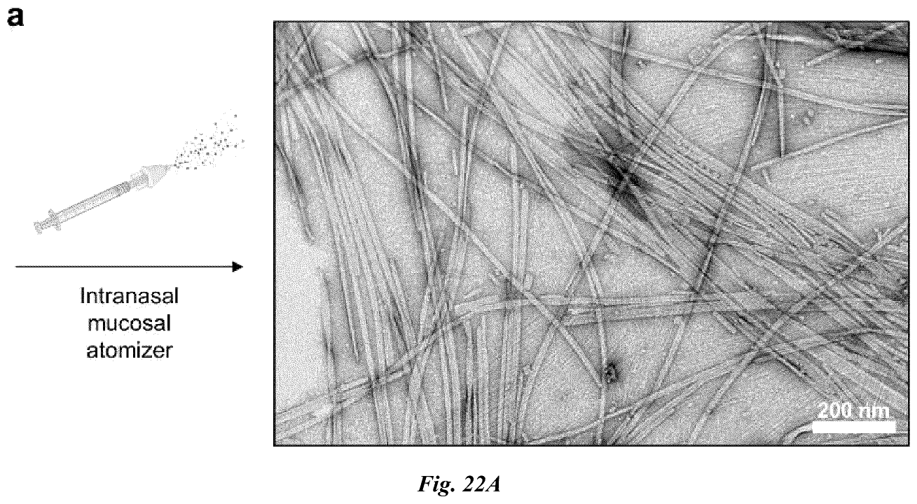

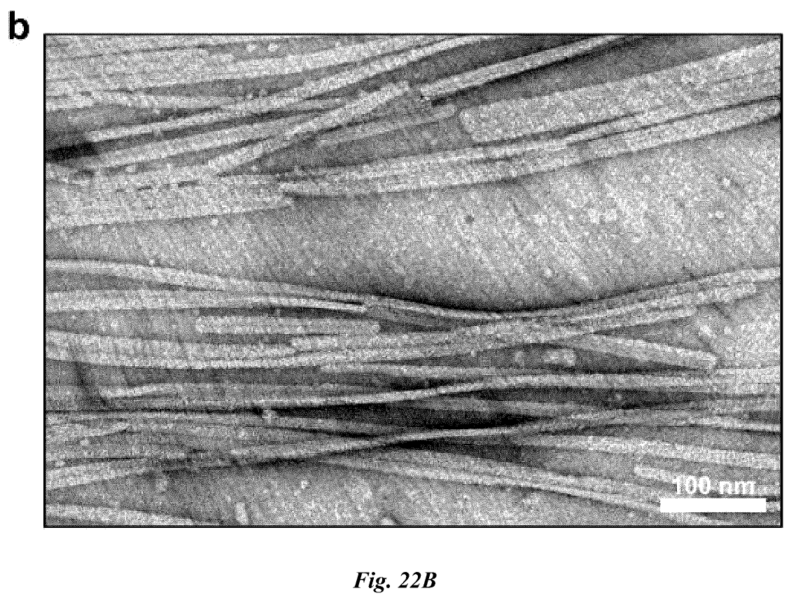

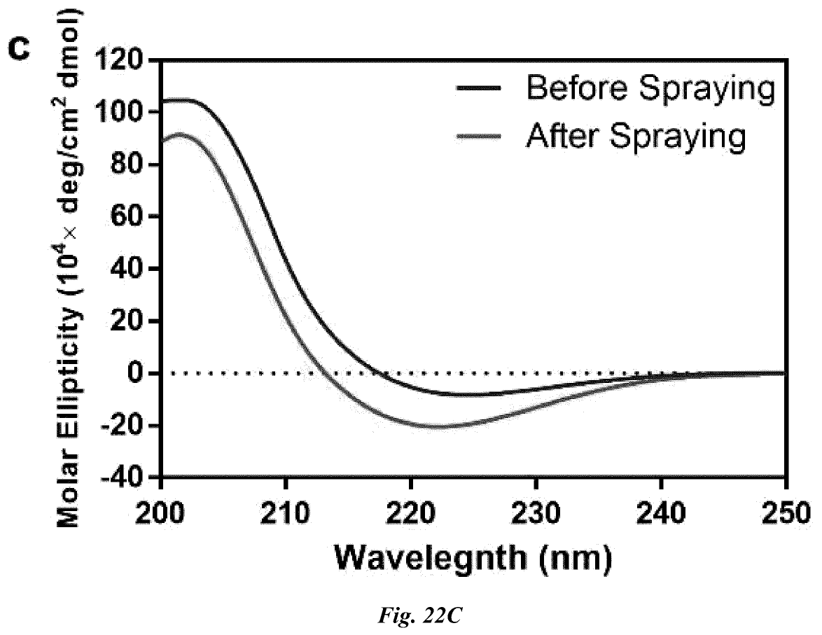

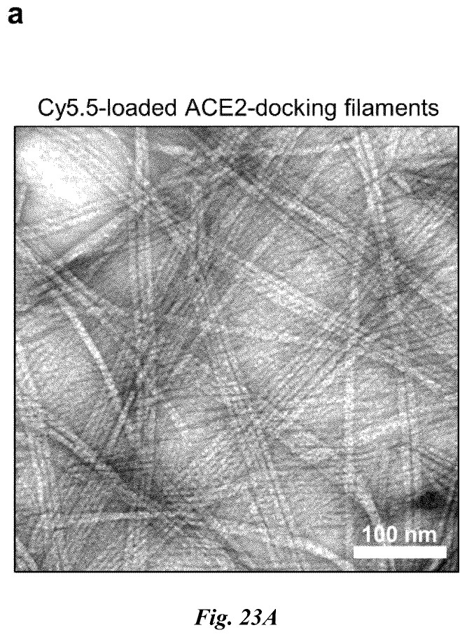

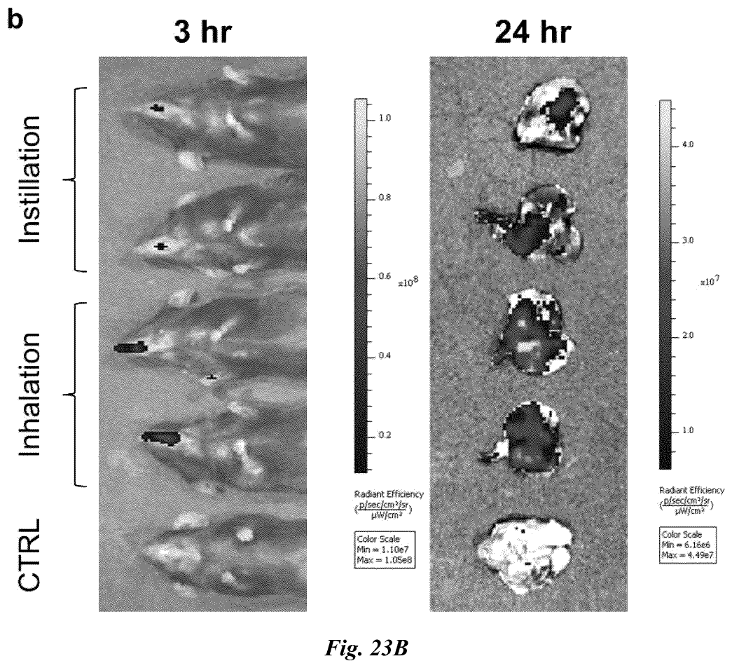

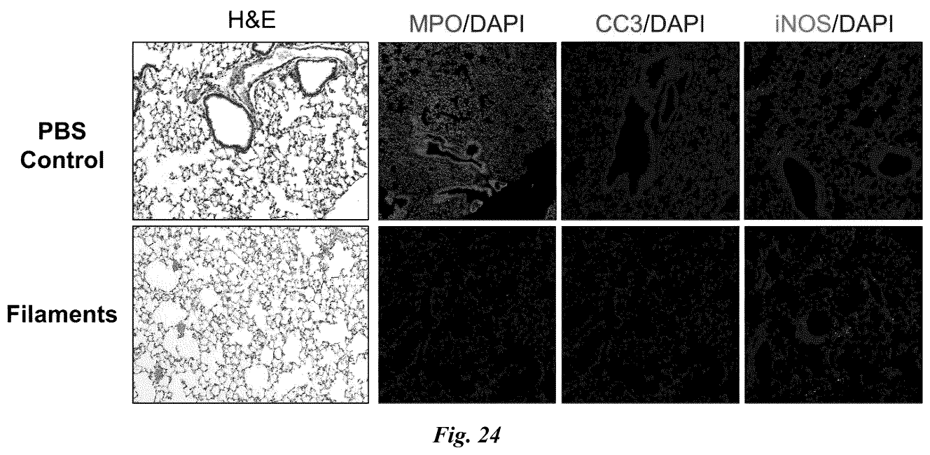

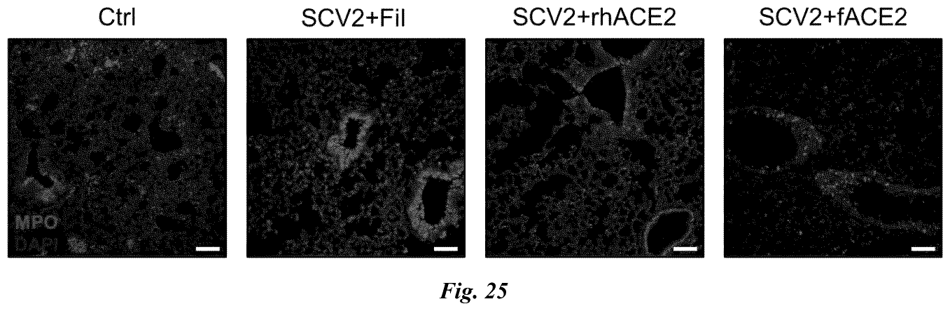

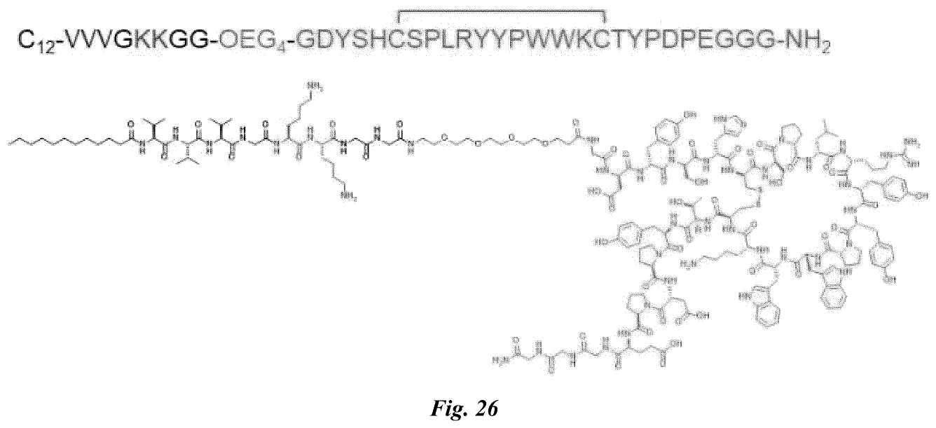

To further elucidate the behavior of fACE2 during aerosol delivery, we next assessed its structural stability during jet nebulization. This is of concern since filaments measure over several microns in length and form from noncovalent interactions, and the resulting size distribution post-nebulization will be critical for achieving ideal distribution and retention of fACE2 within lung tissues.^{39, 49} As observed with TEM, fACE2 maintains its ribbon-like morphology after addition of ACE2 (50 nM), and after jet nebulization, fACE2 maintains its filamentous shape with an observed reduction in contour length, which is expected due to ALI enrichment and shear during aerosol droplet formation (FIG. 3D).³⁶ The influence of these factors is corroborated by CD measurements, where the observed reduction in signal intensity is reflective of weakened hydrogen bonding due to dissociation into smaller structures and/or PA monomers (FIG. 18). The average contour length of fACE2 post-nebulization measured around 343±196 nm, though nebulization does induce polydispersity with respect to filament length (FIG. E). However, this distribution of size could be advantageous with respect to diffusion through the mucus layer atop airway epithelia, as some fACE2 may penetrate the mucus while some remains atop or closer to the mucus layer surface.³⁹ Integration of fACE2 throughout the mucus layer may potentially increase the likelihood of successful virus capture before viral entry into host cells. Furthermore, the degree of filament fragmentation is consistent regardless of formulation concentration (0.2-1 mM filament concentration range), where average contour length measures around 330 nm with similar distributions for each tested concentration (FIG. 19). Moreover, jet nebulizer emission of fACE2 is linear over the course of a nebulization event, with around 6.3%/min (by mass) released of the loaded dose (FIG. 20). Altogether, these data suggest that fACE2 exhibits steady release from a jet nebulizer with consistent size distribution, which is imperative for achieving more uniform distributions within lung tissues for inhalation-based delivery.

1.4.5 Inhibition of Pseudotyped Coronavirus Entry In Vitro by fACE2

Having shown that ACE2-docking filaments bind ACE2 to their surface and carry ACE2 within respirable aerosols, we next evaluated the ability of fACE2 to capture SARS coronavirus (1 and 2) spike protein pseudotyped Feline immunodeficiency virus (FIV) and prevent viral entry in vitro. First, we assessed the cytotoxicity of ACE2-docking filaments against relevant human cell lines (NL20, bronchial epithelial cells; A549, alveolar basal epithelial adenocarcinoma cells; and 293/ACE2/TMPRSS2, stable-producing human ACE2 and co-receptor, TMPRSS2,¹² HEK293 cells, used in following antiviral studies). For all tested concentrations of filaments (0.1-100 μM), cells maintained high viability after treatment (>90%), which is promising for use of this system as a safe delivery vehicle of soluble ACE2 (FIG. 2I). Moreover, the negligible cytotoxicity likely contributes no interference in evaluation of antiviral efficacy in following in vitro assessments.

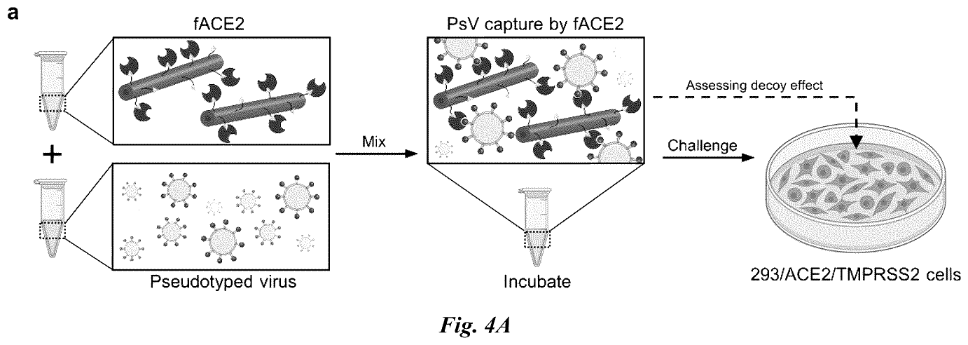

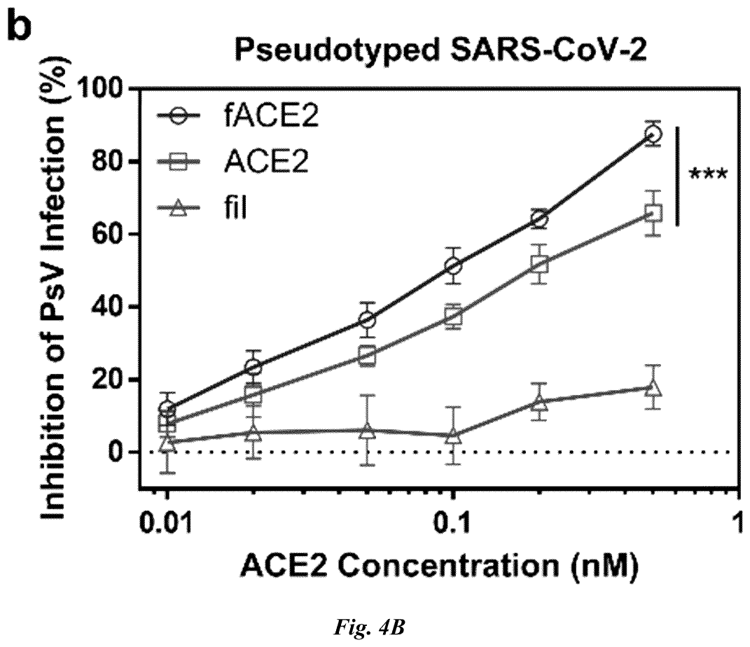

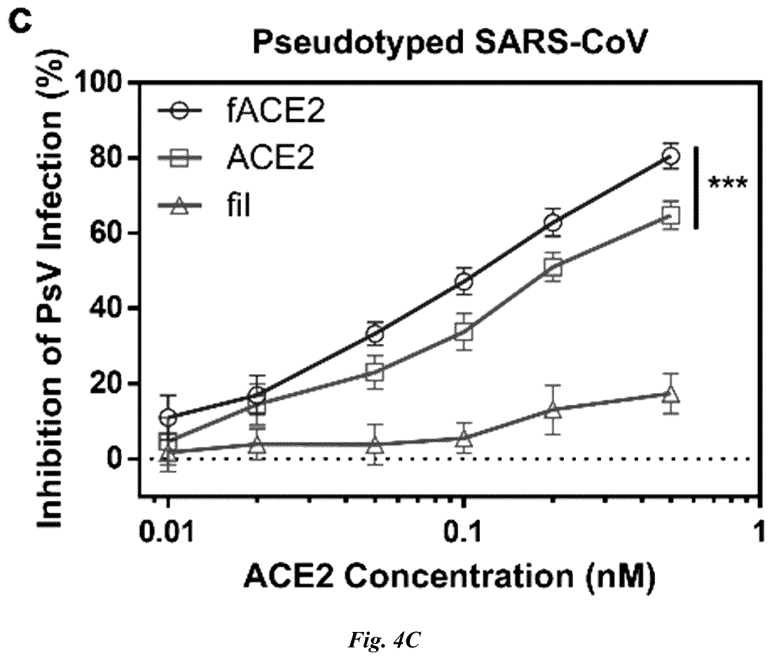

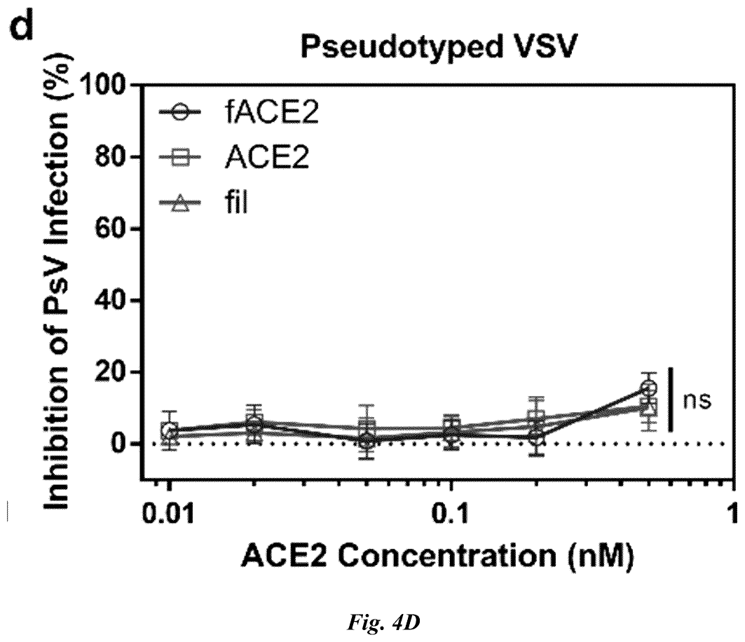

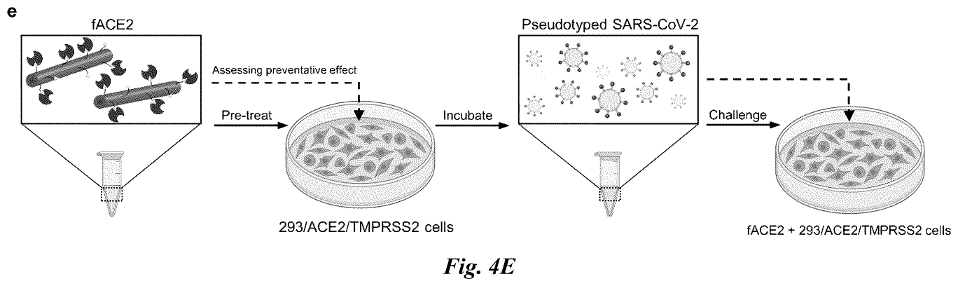

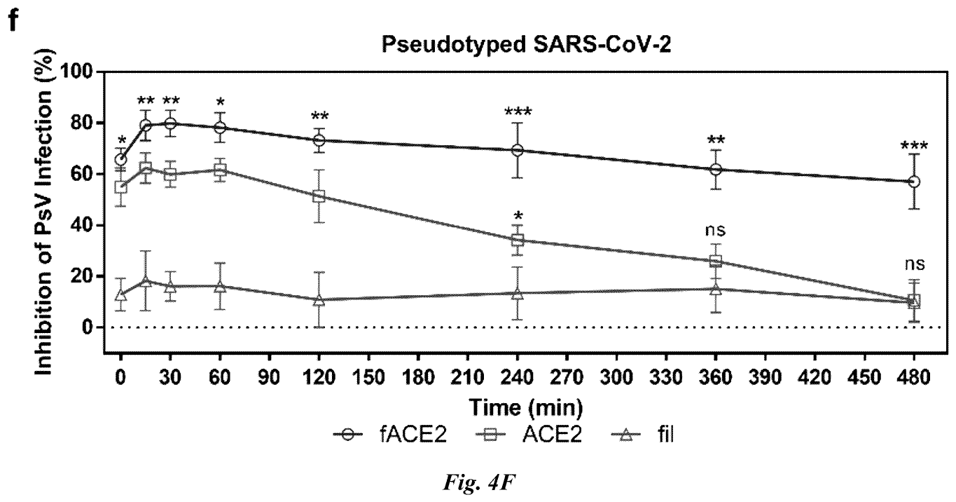

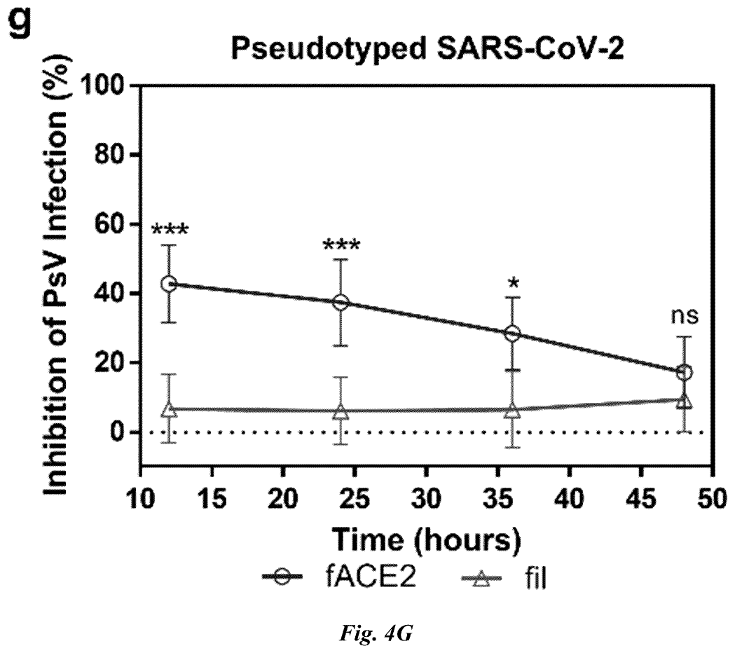

To evaluate the antiviral effect of fACE2, pseudotyped viruses (PsV) were generated for both SARS-CoV-2 and SARS-CoV (alongside vesicular stomatitis virus glycoprotein (VSVG) as a negative control) to yield virus particles decorated with their respective spike protein and containing expression cassette for luciferase to assess viral entry into 293/ACE2/TMPRSS2 cells. We first assessed the decoy effect of fACE2 and relevant controls (free ACE2 and empty ACE2-docking filaments) by pre-incubating varying doses with each PsV and subsequently challenging 293/ACE2/TMPRSS2 cells with the mixture (FIG. 4A). We found that both SARS-CoV-2 and SARS-CoV PsV infection are strongly inhibited by both fACE2 and ACE2 in a dose-dependent manner, suggesting that ACE2 displayed on the surface of fACE2 hijacks S-protein-mediated viral infection and docking of ACE2 does not impede viral capture (FIG. 4B, FIG. 4C). This trend is not observed for the VSVG PsV, as expected, and the empty filaments alone show little to no viral capture for all PsVs tested, emphasizing the key role ACE2 plays in inhibiting S-protein-mediated viral entry. An increase in inhibition is observed at higher concentrations of filaments (˜18% at highest dose against SARS-CoV-2 PsV), which may be due to nonspecific interactions between virus particles and filaments; this may explain the higher PsV infection inhibition observed for fACE2 (˜88%) compared to free ACE2 (˜66%). Against both SARS-CoV-2 and SARS-CoV, fACE2 exhibited potent inhibitory activity, which shows promise in providing broad-spectrum antiviral efficacy for current and future SARS coronaviruses.

Since ACE2 degrades quickly, extending its availability as a decoy is highly desirable for blocking SARS coronavirus infections.¹⁹ Therefore, we next aimed to assess the preventative effect of fACE2 and whether our docking strategy serves to extend the decoy function. To achieve this, we pre-treated 293/ACE2/TMPRSS2 cells with fACE2 and relevant controls (0.5 nM ACE2 dose) and allowed to incubate for a set time before challenge with SARS-CoV-2 PsV (FIG. 4E). We observed that for all tested incubation times, fACE2 exhibited greater inhibitory effect compared to free ACE2; moreover, the extent of inhibition by free ACE2 began to steadily decrease around 2 h and became almost identical to empty filaments by 6 h (FIG. 4F). In stark contrast, fACE2 inhibitory potential declined at a much slower rate, maintaining around ˜60% inhibition of PsV infection at 6 h. The preventative effect was assessed for longer incubation times for fACE2 and empty filaments, where after 12 h, fACE2 preventative efficacy begins to wane and becomes indistinguishable from empty filaments around 36-48 h (FIG. 4G). Altogether, these results highlight a key advantage of fACE2 in preventing viral entry, where it exhibits more potent and sustained inhibitory efficacy compared to free ACE2, likely due to docking to the filament surface. Binding of ACE2 to the filament likely impedes premature degradation and/or cellular uptake of ACE2, thereby enhancing and prolonging its antiviral efficacy.

### 1.4.6 Attenuation of SARS-CoV-2 Viral Loads In Vivo by Inhalation Off ACE2

After demonstrating the enhanced and extended antiviral efficacy afforded by our docking strategy against pseudotyped FIV, we next assessed the efficiency of fACE2 delivery into the lungs and its ability to subsequently capture SARS-CoV-2 in vivo. For delivery to mice, we used an intranasal mucosal atomizer to subject fACE2 to shear forces necessary to generate respirable aerosols before administration, which also yielded filaments of reduced contour length (FIG. 22). By loading ACE2-docking filaments with near-infrared fluorescent dye (Cyanine5.5) to allow for in vivo visualization, we evaluated the distribution and retention of the ACE2-docking filaments administered via both intranasal inhalation and intratracheal instillation into K18-hACE2 transgenic mice. After 3 h post-administration, fluorescence signal is still detectable in nasal passageways of treated mice, evidencing the presence of filaments. Strikingly, after 24 h, excised lungs show strong fluorescence signal throughout the distal lung, suggesting migration and long-term retention of filaments, which may be likely afforded by their filamentous shape (FIG. 23). Histology of lung tissue sections taken at the 24 h time point indicates no obvious signs of structural damage, apoptosis, inflammation, as well as neutrophil infiltration induced by treatment with ACE2-docking filaments compared to PBS control (FIG. 24). Taken together, these results suggest that ACE2-docking filaments exhibit long-term retention within lung tissues after inhalation and are safe, biocompatible delivery vehicles for ACE2.

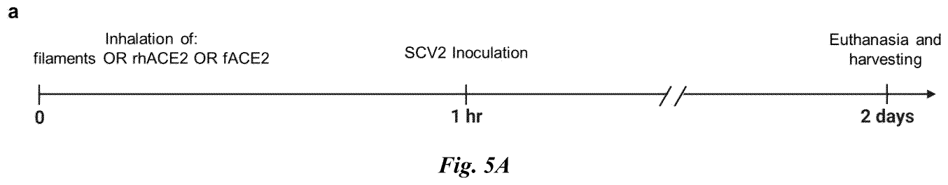

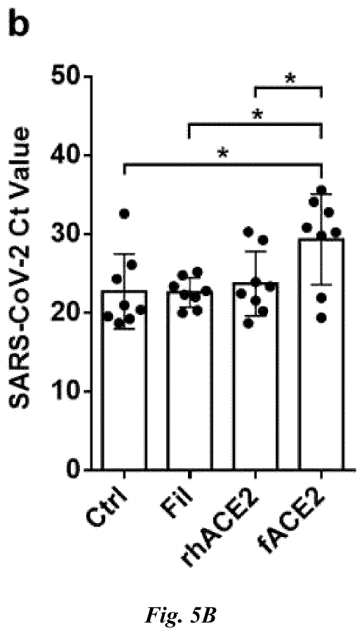

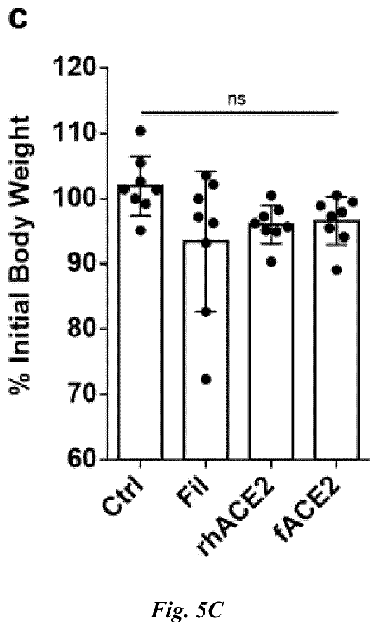

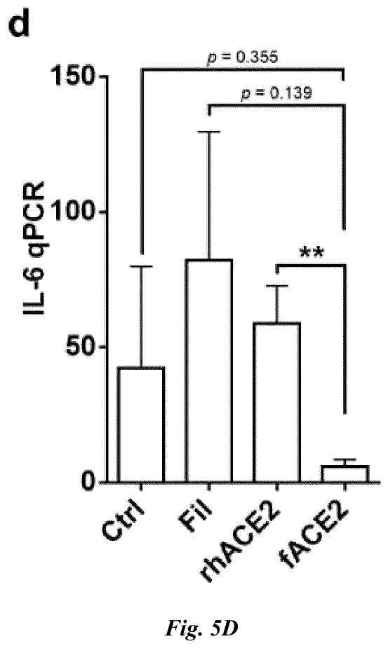

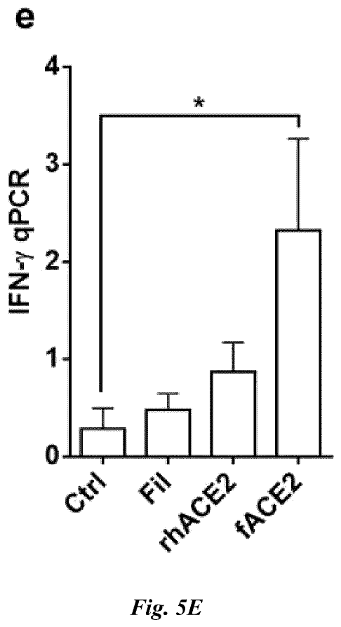

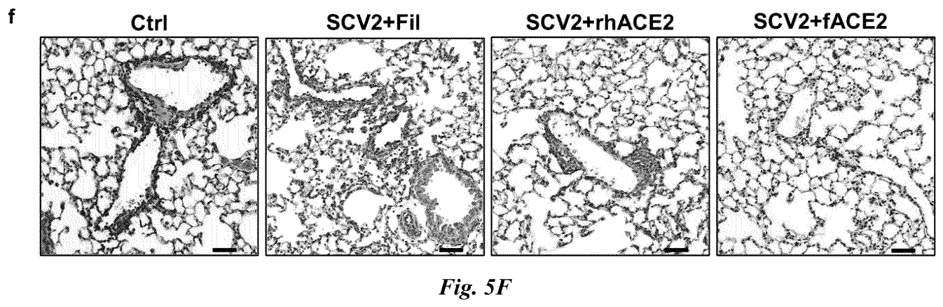

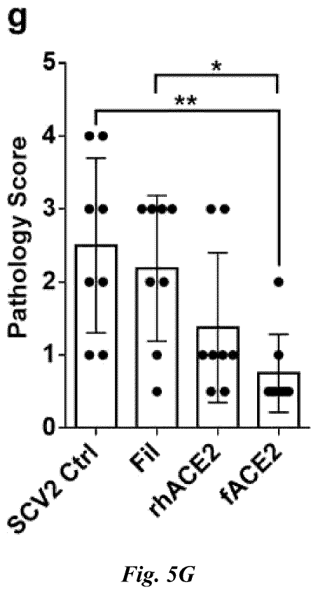

Having achieved successful inhalation delivery of ACE2-docking filaments in vivo, we next assessed the clinical potential of fACE2 as a preventative therapeutic against coronavirus infection. As shown in FIG. 5A, we administered either atomized fACE2 (20 nM dose ACE2), recombinant human ACE2 (20 nM), or empty filaments (equivalent to 20 nM ACE2 dose) to K18-hACE2 mice via intranasal inhalation 1 h before inoculating mice with prototype SARS-CoV-2 virus (USA-WA1/2020, 10⁵ pfu/mouse). After 2 days post-inoculation, the mice were euthanized, and their lung tissues were harvested. As evidenced by increased cycle threshold (Ct) values for N-protein gene expression and reduced N-protein detection in lung tissue, treatment with fACE2 greatly reduces viral load in SARS-CoV-2-infected lungs compared to empty filaments and rhACE2 alone (FIG. 5B, 5H), though the body loss in each group was similar (FIG. 5C). In parallel with the reduced viral load, mice that received treatment with fACE2 also exhibited decreased expression of pro-inflammatory cytokine interleukin-6 (IL-6, FIG. 5D), a hallmark of the hyperinflammatory response and cytokine storm presented in human and animal models of SARS-CoV-2 infection.⁵⁰⁻⁵² Moreover, enhanced expression of antiviral cytokine interferon gamma (IFN-γ, FIG. 5E) was observed, suggesting a restored antiviral immune responses and balanced inflammatory responses, which are typically lacking in progressing COVID-19 patients and animal models and are indicative of severe COVID-19.⁵³⁻⁵⁶ More importantly, fACE2-treated mice displayed alleviated lung inflammation and related pathology, as evidenced by mitigated inflammatory cell infiltration into lung tissues (neutrophil and monocyte, FIG. 5F, FIG. 5G, FIG. 25).⁵⁷ Altogether, these results highlight the prophylactic and therapeutic potential afforded by our docking strategy of decoy ACE2 to filament surfaces to improve antiviral efficacy, clearly illustrating the preventative potential of fACE2 in clinical practice. While preventative efficacy is directly investigated here, these results also highlight the potential of fACE2 as a treatment tool for those already infected with SARS-CoV-2 by reducing the viral load in the airways and distal lungs through trapping newly replicated and released viral particles at the sites of infection.

### 1.5 Materials and Methods

### 1.5.1 Materials

All Fmoc amino acids and resins were purchased from Advanced Automated Peptide Protein Technologies (AAPPTEC, Louisville, KY). The oligoethylene glycol spacer (Fmoc-N-amido-PEG4-acid) was purchased from BroadPharm (San Diego, CA). Recombinant human angiotensin-converting enzyme 2 (ACE2) protein (carrier-free) was purchased from R&D Systems, Bio-Techne (Minneapolis, MN). Biotinylated human recombinant (His-tag) ACE2 protein was purchased from Sino Biological (Wayne, PA). Free DX600 peptide used as a control was purchased from Cayman Chemical (Ann Arbor, MI). Near-infrared fluorescent dye, Cyanine 5.5 carboxylic acid, was purchased from Lumiprobe (Hunt Valley, MD). All other reagents and solvents were sourced from VWR, Avantor (Radnor, PA) or Sigma-Aldrich, MilliporeSigma (St. Louis, MO) without any further purification unless otherwise indicated.

### 1.5.2. Peptide Amphiphile Synthesis and Purification

All peptide amphiphiles (PAs) studied were synthesized using standard 9-fluorenylmethoxycarbonyl (Fmoc) solid phase peptide synthesis techniques. All three PAs (Filler, Ligand, sLigand) were synthesized onto Rink amide 4-methylbenzhydrylamine (MBHA) resin (100-200 mesh, 0.53 mmol/g). All Fmoc deprotections were performed with 20% 4-methylpiperidine in N,N-dimethylformamide (DMF) for 15 minutes, repeated once. After Fmoc removal, each amino acid coupling was performed at a 4:4:6 molar ratio of the Fmoc-protected amino acid, 0-benzotrizole-N,N,N′,N′-tetramethyluronium hexafluorophosphate (HBTU), and N,N-diisopropylethylamine (DIEA) in DMF and shook for 2 h for the coupling reaction. For Ligand and sLigand molecules, Fmoc-PEG₄-COOH was conjugated to peptide chain at a molar ratio of 2:2:3 to resin of Fmoc-PEG₄-COOH, HBTU, and DIEA in DMF and shaken for 24 h. After final amino acid conjugation and Fmoc deprotection, lauric acid (C₁₂ alkyl chain) was coupled to the peptide in a 4:4:6 molar ratio to resin of lauric acid, HBTU, and DIEA in DMF and shaken overnight. For Ligand and sLigand molecules, Acm deprotection and disulfide bond formation (from Cys(Acm) residues of the DX600 peptide ligand segment of the design) was conducted with thallium trifluoroacetate (2 equivalents) in DMF and swirled with resin on ice for 2 h; resin was subsequently washed with methanol (MeOH), DMF, and then MeOH again. Completed PAs were cleaved from their resin by addition of a 10 mL mixture of 95% v/v trifluoroacetic acid (TFA), 2.5% v/v triisopropylsilane (TIS), and 2.5% v/v water and shaken for 3 h. After cleavage, the TFA solution was collected and excess TFA removed via evaporation, and subsequently the product was precipitated with cold diethyl ether and then dried under vacuum overnight.

Each crude PA solid was dissolved in a water and acetonitrile (ACN) mixture containing 0.1% v/v TFA for Ligand and sLigand molecules and 0.1% v/v ammonium hydroxide (NH₄OH) for Filler. A Varian ProStar Model 325 (Agilent Technologies, Santa Clara, CA) reverse-phase high performance liquid chromatography (RP-HPLC) was employed to purify the PA molecules using mobile phases of water and ACN at matching acidic or basic phase. PAs were separated from impurities by preparative RP-HPLC using a Varian PLRP-S column (C₁₈, 100 Å, 10 μm, 150×25 mm) with a flow rate of 20 mL/min, 10 mL injections, and monitoring at 220 nm for peptide absorbance for all molecules. For Filler, the eluent gradient was run linearly from 30% to 45% ACN over 20 min, and for Ligand and sLigand, the gradient was run linearly from 25% to 55% ACN over 30 min. The collected fractions were analyzed by matrix-assisted laser desorption/ionization time of flight (MALDI-ToF) mass spectrometry to isolate fractions containing the molecules of interest. Correct fractions were combined, and excess ACN was removed via rotary evaporation. Samples were then lyophilized using a FreeZone −105° C. 4.5 L freeze dryer (Labconco, Kansas City, MO). Re-characterization of the purified PA powders were conducted by analytical RP-HPLC and MALDI-ToF. Product purity was assessed with analytical RP-HPLC using a Varian Pursuit XRs column (Cis, 5 m, 150×4.6 mm) with a flow rate of 1 mL/min, 20 μL injections, and monitoring at 220 nm. Purity was assessed with a linear gradient of 5% to 95% ACN over 15 min, where area under the curve (AUC) of the PA peak relative to total AUC of all peaks was used to confirm purity greater than 95% (purity data for each molecule shown in FIG. 6, FIG. 7, and FIG. 12). The redissolved PAs were calibrated, aliquoted into cryo-vials, re-lyophilized, and stored at −20° C. freezer for future use. Cheetham et al., Supramolecular Nanostructures Formed by Anticancer Drug Assembly. Journal of the American Chemical Society 135, 2907-2910 (2013); Su et al., Macrocyclization of a Class of Camptothecin Analogues into Tubular Supramolecular Polymers. Journal of the American Chemical Society 141, 17107-17111 (2019); Wang et al. Supramolecular Tubustecan Hydrogel as Chemotherapeutic Carrier to Improve Tumor Penetration and Local Treatment Efficacy. ACS Nano 14, 10083-10094 (2020).

### 1.5.3. MALDI-ToF Mass Spectroscopy

Matrix-assisted laser desorption/ionization time of flight (MALDI-ToF) mass spectroscopy was used to analyze the molecular weights for all synthesized PA molecules with a BrukerAutoflex III MALDI-ToF instrument (Bruker, Billerica, MA). Samples were prepared by first depositing a 2 μL droplet of sinapic acid matrix solution (10 mg/mL in 1:1 v/v water:ACN with 0.05% TFA; Sigma-Aldrich) onto an MTP 384 ground steel target plate (Bruker, Billerica, MA). The matrix was allowed to dry for 5-10 min, and then 1 μL of aqueous PA solution was added to the corresponding spot of dried matrix followed by the immediate addition of 1 μL of matrix solution and mixed. The spots were allowed to dry for 10-20 min before analyzing on the instrument, where samples were irradiated with a 355 nm UV laser and analyzed in the reflectron mode. Representative MALDI-ToF mass spectra for each of the PAs studied are shown in FIG. 6, FIG. 7, and FIG. 12.

### 1.5.4 Molecular Self-Assembly and Co-Assembly of PAs to Form Supramolecular Structures

For self-assembly of PAs, first lyophilized powders of PA were dissolved in 200 μL of hexafluoro-2-propanol (HFIP) to disrupt any preassembled structures. Samples were vortexed and sonicated for 5 min to aid dissolution and mixing. For co-assembled systems, appropriate volumes of Ligand solutions were added to solutions of Filler to achieve specific molar ratios used in this study (Filler:Ligand ranging from 1:1 to 200:1 molar ratio) and tubes of the mixtures were vortexed and sonicated for 5 min to further aid dissolution and mixing of the 2 components. Next, HFIP was evaporated off and dried under vacuum overnight to remove all traces of HFIP. After drying, Milli-Q water or PBS was added to yield the appropriate final concentrations (1 mM for Filler alone and 1 mM Filler all co-assembly systems; 200 μM Ligand and sLigand alone and varied concentration of Ligand relative to Filler for all co-assembly systems) of PAs and then vortexed to aid dissolution. The pH of the solutions was then adjusted with additions of 0.1 M HCl(aq) and 0.1 M NaOH(aq) to yield a final pH of 7.4. The solutions were then heated at 80° C. in a water bath for 1 h to further aid dissolution and facilitate the annealing process and then cooled at room temperature overnight. For docking of ACE2 to filament surfaces, solutions of ACE2 in PBS were mixed with filament solutions at an equal volume and allowed to incubate at room temperature for a set time until use, yielding fACE2.

For encapsulation of near-infrared dye, Cyanine5.5 (Cy5.5 carboxylic acid, Lumiprobe, Hunt Valley, MD), into hydrophobic filament cores, the same procedure was followed as described above, where Cy5.5 was dissolved with filament components in HFIP at 2:1 molar excess relative to Filler. After removal of HFIP, dissolution in PBS, annealing, and aging overnight, unencapsulated Cy5.5 (precipitated) was removed by centrifugation (13,400 rpm, 3 min), and filament-containing supernatant removed for further analysis and use for in vivo lung distribution studies.

### 1.5.5 Transmission Electron Microscopy

Solutions of PAs were added (10 μL drop) onto a carbon film copper grid (400 square mesh, Electron Microscopy Sciences, Hatfield, PA) and allowed to sit for 1 min. Afterward, the excess solution was wicked away with filter paper to leave a thin film of the sample atop the grid. Subsequently to achieve negative staining, a 7 μL droplet of uranyl acetate solution (2 wt % in Milli-Q water) was added atop the grid and blotted away after 30 seconds. Grids were left to dry for at least 3 h before imaging on a FEI Tecnai 12 TWIN transmission electron microscope (100 kV acceleration voltage). All images were recorded using a SIS Megaview III wide-angle CCD camera. Filament diameters and contour lengths were measured using ImageJ software (NIH, Bethesda, MD), where a minimum of 35 individual structures were analyzed for diameter lengths and a minimum of 350 separate filamentous structures were analyzed for contour length measurements.

### 1.5.6 Cryogenic Transmission Electron Microscopy

Lacey carbon-coated copper grids (Electron Microscopy Sciences, Hatfield, PA) were treated with plasma air for 30 seconds before sample preparation to render the lacey carbon film hydrophilic. Sample addition to the grids was achieved with a VitroBot with a controlled humidity chamber (FEI, Hillsboro, OR) maintained at 95% humidity. Droplets of sample solutions (in PBS at pH=7.4, 6 μL) were added to suspended grids in the Vitrobot and allowed to sit for 1 min before the grid was blotted with filter paper using Vitrobot preset parameters and then immediately plunged into a liquid ethane reservoir precooled by liquid nitrogen to produce a think vitreous ice film on the surface of the grid. Grids were then transferred to a cryo-holder and cryo-transfer stage that were also cooled by liquid nitrogen. All imaging was performed on a FEI Tecnai 12 Twin transmission electron microscope, operating at a 100 kV acceleration voltage. The cryo-holder temperature was maintained below −170° C. with liquid nitrogen to prevent the sublimation of vitreous water during imaging. All images were acquired with a 16-bit 2K×2K FEI Eagle bottom-mount camera.

### 1.5.7 Dynamic Light Scattering (DLS)

Spherical micelle/aggregate solutions of Ligand and sLigand dissolved in PBS at pH=7.4 were diluted to 100 μM and placed in a UV-transparent disposable cuvettes (0.5 mL, 45×12 mm, special plastic, Sarstedt, Numbrecht, Germany). Samples were analyzed on a Malvern Zetasizer Nano-ZS ZEN3600 (Malvern Panalytical) at 25° C. Three runs were collected for each sample (10 scans/measurement) and averaged.

### 1.5.8 Circular Dichroism (CD)

Solutions of the supramolecular systems were diluted to 100 μM, added to a 1 mm path length quartz UV-Vis absorption cell (Thermo Fisher Scientific, Pittsburgh, PA), and then analyzed with a Jasco J-710 spectropolarimeter (JASCO, Easton, MD). Each sample was analyzed by three repeated scans from wavelengths of 190 to 300 nm (for samples in water) or from 200 to 300 nm (for samples in PBS). The high tension (HT) values were monitored during run collection (ranging from 200 to 600 V) to ensure no scattering artifacts were present in the representative spectra. A background spectrum (water or PBS) was obtained and subtracted from sample spectra. All obtained spectra were averaged over 3 scans and converted from ellipticity (mdeg) to molar ellipticity (deg·cm²·dmol⁻¹). For nebulized samples, sample concentration post-nebulization was determined using a standard curve derived from analytical HPLC runs to account for solvent evaporation that may occur during aerosol formation.

### 1.5.9 Critical Micelle Concentration (CMC) Measurements

Using a Nile Red assay, the CMC of each PA monomer (Filler and Ligand) and their co-assembly (1:1 molar ratio) was determined. Nile Red dye intensely fluoresces within hydrophobic environments but is strongly quenched and red-shifted in aqueous environments. Thus, when mixed with PAs at a concentration exceeding the CMC, Nile Red will embed within the hydrophobic core and emit a strong fluorescence signal. A 500 μM stock solution of Nile Red was prepared in acetone, and 10 μL of the stock was added to microcentrifuge tubes. Acetone was evaporated off in the dark. Solutions of the samples (500 μL, water) were added to the tubes at varied concentrations and were aged for 2 days in the dark at room temperature. Samples were added to a quartz micro fluorometer cell (0.7 mL, 10 mm pathlength, Starna Cells, Atascadero, CA), and at an excitation wavelength of 550 nm, five parallel emission spectra were recorded at a wavelength range of 580 to 720 nm on a Duetta UV-Vis-NIR spectrofluorometer (HORIBA Scientific). The emission intensity ratio for each run at 635 nm (emission maximum of Nile Red in a hydrophobic environment) to 660 nm (emission maximum of Nile Red in a hydrophilic environment) was plotted against tested concentrations to obtain a transition curve from which CMC was determined.

### 1.5.10 Bio-Layer Interferometry (BLI)

Biotinylated human ACE2 (NP_068576.1, Sino Biological) was immobilized to streptavidin-coated tips (Pall Life Sciences) for analysis on an Octet Red96 bio-layer interferometry (BLI) instrument (Sartorius). Less than 5 signal units (nm) of ACE2 was immobilized to minimize mass transfer effects. PBSA (PBS pH 7.2 containing 1% BSA) was used for all dilutions and as dissociation buffer. Tips were exposed to serial dilutions of Ligand PA and sLigand PA in a 96-well plate for 300 s. Dissociation was then measured for 150 s. Surface regeneration for all interactions was conducted using 15 s exposure to 0.1 M glycine pH 3.0 solution. Normalized equilibrium binding curves were obtained by plotting the response value at the end of the association phase for each sample dilution, dividing by the molecular weight of each ligand, and normalizing to the maximum value. Equilibrium binding curves were fitted and K_{D} values determined using GraphPad Prism data analysis software v9.0, assuming all binding interactions to be first order. Experiments were performed twice with similar results.

### 1.5.11 ACE2 Activity Assays

Fluorogenic peptide substrate, Mca-YVADAPK(Dnp)-OH (Mca: (7-methoxycoumarin-4-yl)acetyl, Dnp: 2,4-dinitrophenyl; R&D Systems, Bio-Techne, Minneapolis, MN), was diluted from stock (4 mM) to a final concentration of 1 mM in dimethyl sulfoxide (DMSO). All monitored reactions with ACE2 were conducted in black, 96-well, flat bottom, tissue culture-treated microplates (Falcon, Corning, NY) in 100 μL of PBS (pH=7.4) at room temperature with substrate (1-5 μL, 10-50 μM final concentration, DMSO concentration maintained ≤5% v/v) added immediately before measurement. Equal volume of ACE2 was added to solutions of the supramolecular structure and incubated for 1 h for all experiments unless stated otherwise. ACE2 activity was monitored continuously (every 5 min for 2 h total) by measuring fluorescence intensity (λₑₓ=320 nm, λₑₘ=405 nm) upon substrate hydrolysis using a SpectraMax M3 microplate reader (Molecular Devices LLC, San Jose, CA). The initial velocity for each reaction was determined from the rate of fluorescence evolved over the 5-20 min time course (slope from linear regression analysis of this region). The extent of enzymatic inhibition (used as a reflection of the docking efficiency of ACE2 to filament surface) was determined as the measured initial velocity of a tested condition relative to free ACE2 at the same ACE2 and substrate concentration.

### 1.5.12 Zeta Potential Measurements

All zeta potential measurements were conducted for samples in PBS (pH=7.4) at 25° C. Samples were added to a disposable folded capillary zeta cell (DTS1070, Malvern Panalytical) and analyzed using a Malvern Zetasizer Nano-ZS ZEN3600 (Malvern Panalytical). For analysis of mixtures, ACE2 was added to solutions of Ligand, sLigand, and 20:1 docking filaments (molar ratio of Filler:Ligand) at an equal volume, such that a final concentration of 50 nM ACE2 was achieved, alongside 50 μM Ligand or sLigand. Three repeated runs were performed for each sample (20 measurements/run) and then averaged. The intensity-averaged zeta potential for mixtures of samples was calculated based on the average derived count rate and zeta potential measured for the individual components and their respective volume fractions after mixing assuming no interaction. This theoretical value was compared to measured experimental values of the systems after ACE2 addition to invalidate the “no interaction” assumption; these calculations were conducted only for Ligand and sLigand spherical aggregates with ACE2, since filaments are anisotropic and more polydisperse and Zetasizer software uses models for fitting best suited to spherical particles. Tantakitti et al., Energy landscapes and functions of supramolecular systems. Nature Materials 15, 469-476 (2016); Chen et al., Self-Repair of Structure and Bioactivity in a Supramolecular Nanostructure. Nano Letters 18, 6832-6841 (2018).

### Intensity-Averaged Zeta Potential: ξ_{AB}

For a mixture of 2 components: component A (volume fraction, α; scattering intensity/derived count rate, int_{A}; zeta potential, ξ_{A}) and component B (volume fraction, β; scattering intensity/derived count rate, int_{B}; zeta potential, ξ_{B})

$$α + β = 1 \\ {ζ}_{AB} = \frac{α \cdot {int}_{A} \cdot {ζ}_{A} + β \cdot {int}_{B} \cdot {ζ}_{B}}{α \cdot {int}_{A} + β \cdot {int}_{B}}$$

For ACE2+Ligand (equal volume fractions): calculated ξ_{AB}=−17.6 mV, measured −25.1 mV
For ACE2+sLigand (equal volume fractions): calculated ξ_{AB}=−19.0 mV, measured −19.2 mV
1.5.13 Aerosolization of ACE2-Docking Filaments and fACE2

For studies involving the jet nebulization of ACE2-docking filaments and fACE2, solutions of these (3 mL) were added to a disposable jet nebulizer (Neb Kit 500, Drive Medical, Port Washington, NY) and connected to a nebulizer compressor (Rite-Neb 4, ProBasics, Marlboro, NJ) for constant air flow supply (˜6-10 Lpm). The emitted mist was collected by fitting the outlet of the nebulizer with a 50 mL conical tube, where liquid aerosol droplets condense on the walls of the tube. Afterward, the conical tubes were centrifuged at 4000 rpm for 3 min and the collected solution was removed for further analysis. For quantification of ACE2 activity post-nebulization, emitted mist solutions of fACE2 were dialyzed against PBS for 48 h using a Spectra/Por Float-A-Lyzer G2 dialysis device (MWCO: 20 kDa; Spectrum Labs, Rancho Dominquez, CA) to facilitate ACE2 separation from filaments before analysis.

For the release rate of ACE2-docking filaments from the nebulizer, the device was weighed before and after addition of filament solutions (3 mL). For the course of a 10 min nebulization event, solution in the reservoir (50 μL) was collected at 2 min intervals and the nebulizer with remaining solution were weighed and mass recorded. Using analytical HPLC, the collected reservoir samples were assessed to determine the concentration of filaments leftover at each time point. Using gravimetric data and the analyzed concentrations, release profiles were determined for the filaments via mass balance analysis.

For in vivo studies involving inhalation delivery of ACE2-docking filaments to mice, filament solutions were loaded into a BD Luer-Lok™ 1 mL syringe, which was then outfitted with a MAD Nasal™ intranasal mucosal atomization device (Teleflex Medical, Morrisville, NC) to emit liquid droplets by pushing solution through the syringe. Collected solutions were used for subsequent administration to mice.

### 1.5.14 Cell Lines

The A549 human alveolar epithelial adenocarcinoma cell line was supplied from ATCC (CCL-185) and grown in F12-K media supplemented with 10% v/v fetal bovine serum (FBS) and 1% v/v penicillin/streptomycin (Gibco, Invitrogen). The NL20 human bronchial epithelial cell line was supplied from ATCC (NCI-PBCF-CRL2503) and grown in F12-K media supplemented with 4% v/v FBS and 1% v/v penicillin/streptomycin (Gibco, Invitrogen) alongside additional insulin (0.005 mg/mL), epidermal growth factor (10 ng/mL), transferrin (0.001 mg/mL), and hydrocortisone (500 ng/mL). The stable ACE2/TMPRSS-expressing human embryonic kidney HEK293 cell line was kindly provided by Dr. Marc Johnson lab (University of Missouri School of Medicine) and grown in DMEM media supplemented with 10% v/v FBS and 1% v/v penicillin/streptomycin (Gibco, Invitrogen). All cells were grown in 75 cm² cell culture flasks (Falcon, Corning) incubated at 37° C. and 5% CO₂ in a humidified Heracell VIOS 160i incubator (ThermoFisher Scientific).

### 1.5.15 Cell Viability

Cells were seeded onto 96-well flat-bottom, tissue culture-treated plate (Falcon) at a density of 5000 cells/well and incubated for 24 h. The cells were then treated with varying concentrations of ACE2-docking filaments (20:1 molar ratio of Filler:Ligand, in 1×PBS at pH=7.4), ranging from 0.1 to 100 μM, and then incubated for an additional 48 h. Cell viability was assessed with an MTT assay (Invitrogen) according to the manufacturer's protocols. Experiments were performed with 5 technical repeats for each condition with a total of 3 biological repeats.

### 1.5.16 Inhibition of Pseudotyped Virus (PsV) Infection In Vitro

Both SARS-CoV and SARS-CoV-2 S protein cDNA (gift from Dr. Marc Johnson, University of Missouri School of Medicine) was used to pseudotype Feline immunodeficiency virus (FIV) expressing luciferase by using previously described methods. Johnson et al., Optimized Pseudotyping Conditions for the SARS-COV-2 Spike Glycoprotein. Journal of Virology 94, e01062-01020 (2020). A vesicular stomatitis virus G (VSV G) protein pseudotyped FIV expressing luciferase was used as positive control for viral transduction.

For the dose-response decoy effect studies (FIG. 4A, FIG. 4B, FIG. 4C, FIG. 4D), prior to infection, 2 μL of PsV (PsV titers: SARS-CoV-2, 1.7×10¹⁴ VP/mL; SARS-CoV, 1.7×10¹⁴ VP/mL; VSV, 4.0×10¹⁴ VP/mL) was added to 100 μL of Opti-MEM media (Gibco, Invitrogen) supplemented with 2% v/v FBS, where 10 μL of fACE2, free ACE2, or empty ACE2-docking filaments (in 1×PBS at pH=7.4) were then added to achieve final desired concentration of ACE2 (or equivalent dose for empty filaments). The mixture was incubated at 37° C. for 45 min. Afterward, 100 μL of the mixture was transferred to the target cells (293/ACE2/TMPRSS2, >90% confluency) in 24-well flat-bottom, tissue culture-treated plate (Falcon). Cells were the incubated for an additional hour, and afterwards the culture media was changed to fresh media. After an additional 48 h incubation, the Luciferase Assay System kit (Promega) was used to analyze luciferase activity following the manufacturer's protocol. Experiments were performed with 6 technical repeats for each condition with a total of 3 biological repeats.

For assessment of the preventative effect of fACE2 (FIG. 4E, FIG. 4F, FIG. 4G), 293/ACE2/TMPRSS2 cells (>90% confluency) in a 24-well flat-bottom, tissue-culture-treated plates were treated with 100 μL of Opti-MEM media supplemented with 2% v/v FBS containing fACE2, ACE2, or empty ACE2-docking filaments (at 0.5 nM ACE2 dose or its equivalent, in 1×PBS at pH=7.4). The cells with added therapeutic were incubated at 37° C. for set time points (ranging from 0 min to 8 h for all 3 groups, additional 12-48 hr for fACE2 and empty filament groups) before being challenged by addition of SARS-CoV-2 PsV (2 μL, titer: 1.7×10¹⁴ VP/mL). With the added PsV, cells were incubated for an additional hour, and afterward, the culture media was replaced with fresh media. After an additional 48 h incubation, luciferase activity was assessed with the Luciferase Assay System kit (Promega) following manufacturer's protocols. Experiments were performed with 6 technical repeats for each condition with a total of 3 biological repeats.

### 1.5.17 Animal Studies

K18-hACE2 mice (male and female, 8-16 weeks old; Jax Lab) were utilized for all animal experiments, which were approved by the JHU Animal Care and Use Committee. The animals were housed individually with access to food, water, and cage enrichment. After 1 week of acclimatization in the animal biosafety level-3 (ABSL-3) facility, the animals were anesthetized with ketamine and xylazine for intranasal instillation of 20 μL of either PBS, filament, recombinant human ACE2 (20 nM) or formulated recombinant human ACE2 (fACE2, 20 nM). 1-hour post reagent administration, mice were inoculated with 1.5×105 50% tissue culture infectious dose (TCID50) of SARS-CoV-2 (USA-WA1/2020), delivered in 30 μL of DMEM. Mice were monitored daily for sign of sepsis, casualty. All mice were sacrificed two days post SARS-CoV-2 infection, and lung tissues were collected for further analysis.

### 1.5.18 In Vivo Imaging System (IVIS) Fluorescence Imaging

To execute the imaging, mice were anesthetized with Ketamine/Xylene, and given Cy5.5-loaded filaments through nasal inhalation. 24 hour-post inhalations, mice were sacrifice and whole lung was isolated. The lung fluorescence image was performed in the IVIS 100 system for 2-5 min at high sensitivity. Regions of interest was identified and quantified using Living Image software (Caliper).

### 1.5.19 Histopathology and Immunofluorescence

Formalin-fixed and paraffin-embedded tissue sections were stained with hematoxylin and eosin (H&E) or anti-SARS-CoV-2 N protein antibody (Novus, NB100-56576; at a dilution of 1:200). Morphometric analyses were performed on affected lung tissues using Image J software (NIH, USA). A minimum of three fields of view were obtained from each animal (n=8 animals; 4 male and 4 female). Heat-induced epitope retrieval was conducted by heating slides to 95° C. for 20 min in sodium citrate-based ER1 buffer (Leica Biosystems, Richmond, IL) before immunostaining. Immunostaining was performed using the Bond RX automated system (Leica Biosystems, Richmond, IL). Positive immunostaining was visualized using immunofluorescences (IF).

### 1.5.20 RT-qPCR

Total mouse lung RNA was isolated using Trizol reagent (Life Technologies) following the manufacturer's protocols. RNA was reverse transcribed using iScript cDNA Synthesis kit from Bio-Rad. SARS-CoV-2 N gene expression was determined by quantitative Taqman PCR (IDT) following the protocols set by the manufacturer. The cycle threshold (Ct) values were normalized by the Ct value of a housekeeping gene GAPDH (Bio-Rad). All other genes expression was determined by SyBr green RT-qPCR as described in our previous publication. Sodhi et al., A Dynamic Variation of Pulmonary ACE2 Is Required to Modulate Neutrophilic Inflammation in Response to Pseudomonas aeruginosa Lung Infection in Mice. The Journal ofImmunology 203, 3000 (2019).

### 1.6 Summary

In this work, we demonstrate the development of peptide-based supramolecular filaments for delivery of ACE2 in inhalable aerosols to capture SARS coronaviruses and prevent infection. Through incorporation of a peptide that inhibits carboxypeptidase activity of ACE2 into our design, we are able to dock ACE2 to the surface of supramolecular filaments through enzyme-substrate complexation, while leaving the spike protein RBD-binding site exposed. Importantly, this docking strategy enables us to silence ACE2's enzymatic activities, while also stabilizing ACE2 for nebulization and inhalable delivery and increasing its retention at the mucosal layer of lung tissue when inhaled as a respirable aerosol. We demonstrate that fACE2 can act as a decoy for viral binding, as evidenced by enhanced and prolonged reduction of SARS-CoV-2 viral load in vitro and in vivo, and this reduction in viral load is able to prevent lung damage. Taken together, these studies establish that our novel fACE2 system has high translational potential to prevent current and future coronavirus infections.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
1. Wang, C., Horby, P. W., Hayden, F. G. & Gao, G. F. A novel coronavirus outbreak of global health concern. The Lancet 395, 470-473 (2020).
2. Zhou, P. et al. A pneumonia outbreak associated with a new coronavirus of probable bat origin. Nature 579, 270-273 (2020).
3. Tay, M. Z., Poh, C. M., Renia, L., MacAry, P. A. & Ng, L. F. P. The trinity of COVID-19: immunity, inflammation and intervention. Nature Reviews Immunology 20, 363-374 (2020).
4. Wang, Y. et al. Remdesivir in adults with severe COVID-19: a randomised, double-blind, placebo-controlled, multicentre trial. The Lancet 395, 1569-1578 (2020).
5. Williamson, B. N. et al. Clinical benefit of remdesivir in rhesus macaques infected with SARS-CoV-2. Nature 585, 273-276 (2020).
6. Hacisuleyman, E. et al. Vaccine Breakthrough Infections with SARS-CoV-2 Variants. New England Journal of Medicine (2021).
7. To, K. K.-W. et al. Coronavirus Disease 2019 (COVID-19) Re-infection by a Phylogenetically Distinct Severe Acute Respiratory Syndrome Coronavirus 2 Strain Confirmed by Whole Genome Sequencing. Clinical Infectious Diseases (2020).
8. Li, Q. et al. The Impact of Mutations in SARS-CoV-2 Spike on Viral Infectivity and Antigenicity. Cell 182, 1284-1294.e1289 (2020).
9. Zhang, L. et al. SARS-CoV-2 spike-protein D614G mutation increases virion spike density and infectivity. Nature Communications 11, 6013 (2020).
10. Kuba, K. et al. A crucial role of angiotensin converting enzyme 2 (ACE2) in SARS coronavirus-induced lung injury. Nature Medicine 11, 875-879 (2005).
11. Lan, J. et al. Structure of the SARS-CoV-2 spike receptor-binding domain bound to the ACE2 receptor. Nature 581, 215-220 (2020).
12. Hoffmann, M. et al. SARS-CoV-2 Cell Entry Depends on ACE2 and TMPRSS2 and Is Blocked by a Clinically Proven Protease Inhibitor. Cell 181, 271-280 e278 (2020).
13. Yan, R. et al. Structural basis for the recognition of SARS-CoV-2 by full-length human ACE2. Science 367, 1444 (2020).
14. Zoufaly, A. et al. Human recombinant soluble ACE2 in severe COVID-19. The Lancet Respiratory Medicine 8, 1154-1158 (2020).
15. Yeung, M. L. et al. Soluble ACE2-mediated cell entry of SARS-CoV-2 via interaction with proteins related to the renin-angiotensin system. Cell 184, 2212-2228.e2212 (2021).
16. Chan, K. K. et al. Engineering human ACE2 to optimize binding to the spike protein of SARS coronavirus 2. Science 369, 1261 (2020).
17. Glasgow, A. et al. Engineered ACE2 receptor traps potently neutralize SARS-CoV-2. Proceedings of the National Academy of Sciences 117, 28046 (2020).
18. Monteil, V. et al. Inhibition of SARS-CoV-2 Infections in Engineered Human Tissues Using Clinical-Grade Soluble Human ACE2. Cell 181, 905-913.e907 (2020). 19. Jia, H. Pulmonary Angiotensin-Converting Enzyme 2 (ACE2) and Inflammatory Lung Disease. Shock 46 (2016).
20. Jia, H., Neptune, E. & Cui, H. Targeting ACE2 for COVID-19 Therapy: Opportunities and Challenges. American Journal of Respiratory Cell and Molecular Biology 64, 416-425 (2020).
21. Liu, P. et al. Novel ACE2-Fc chimeric fusion provides long-lasting hypertension control and organ protection in mouse models of systemic renin angiotensin system activation. Kidney Int 94, 114-125 (2018).
22. Haschke, M. et al. Pharmacokinetics and pharmacodynamics of recombinant human angiotensin-converting enzyme 2 in healthy human subjects. Clin Pharmacokinet 52, 783-792 (2013).
23. Zhang, Q. et al. Cellular Nanosponges Inhibit SARS-CoV-2 Infectivity. Nano Letters 20, 5570-5574 (2020).
24. Rao, L. et al. Decoy nanoparticles protect against COVID-19 by concurrently adsorbing viruses and inflammatory cytokines. Proc Natl AcadSci USA 117, 27141-27147 (2020).
25. Li, Z. et al. Cell-mimicking nanodecoys neutralize SARS-CoV-2 and mitigate lung injury in a non-human primate model of COVID-19. Nature Nanotechnology (2021).
26. Yu, Y.-C., Berndt, P., Tirrell, M. & Fields, G. B. Self-Assembling Amphiphiles for Construction of Protein Molecular Architecture. Journal of the American Chemical Society 118, 12515-12520 (1996).
27. Hartgerink, J. D., Beniash, E. & Stupp, S. I. Self-Assembly and Mineralization of Peptide-Amphiphile Nanofibers. Science 294, 1684 (2001).
28. Hartgerink, J. D., Beniash, E. & Stupp, S. I. Peptide-amphiphile nanofibers: A versatile scaffold for the preparation of self-assembling materials. Proceedings of the National Academy of Sciences 99, 5133 (2002).
29. Zhang, P., Cheetham, A. G., Lin, Y.-a. & Cui, H. Self-Assembled Tat Nanofibers as Effective Drug Carrier and Transporter. ACS Nano 7, 5965-5977 (2013).
30. Hudalla, G. A. et al. Gradated assembly of multiple proteins into supramolecular nanomaterials. Nature Materials 13, 829-836 (2014).
31. Lee, S. S. et al. Gel Scaffolds of BMP-2-Binding Peptide Amphiphile Nanofibers for Spinal Arthrodesis. Advanced Healthcare Materials 4, 131-141 (2015).
32. Miller, S. E. et al. Electrostatically Driven Guanidinium Interaction Domains that Control Hydrogel-Mediated Protein Delivery In Vivo. ACS Central Science 5, 1750-1759 (2019).
33. Li, Y. et al. Bioinspired supramolecular engineering of self-assembling immunofibers for high affinity binding of immunoglobulin G. Biomaterials 178, 448-457 (2018).
34. Krishnamurthy, S. et al. Engineered amphiphilic peptides enable delivery of proteins and CRISPR-associated nucleases to airway epithelia. Nature Communications 10, 4906 (2019).
35. Si, Y. et al. Adjuvant-free nanofiber vaccine induces in situ lung dendritic cell activation and T_{H}17 responses. Science Advances 6, eaba0995 (2020).
36. Anderson, C. F., Chakroun, R. W., Su, H., Mitrut, R. E. & Cui, H. Interface-Enrichment-Induced Instability and Drug-Loading-Enhanced Stability in Inhalable Delivery of Supramolecular Filaments. ACS Nano 13, 12957-12968 (2019).
37. Geng, Y. et al. Shape effects of filaments versus spherical particles in flow and drug delivery. Nature Nanotechnology 2, 249-255 (2007).
38. Edwards, D. A. et al. Large Porous Particles for Pulmonary Drug Delivery. Science 276, 1868 (1997).
39. Lai, S. K. et al. Rapid transport of large polymeric nanoparticles in fresh undiluted human mucus. Proceedings of the National Academy of Sciences 104, 1482 (2007).
40. Towler, P. et al. ACE2 X-ray structures reveal a large hinge-bending motion important for inhibitor binding and catalysis. J Biol Chem 279, 17996-18007 (2004).
41. Li, Y. et al. Selective Capture and Recovery of Monoclonal Antibodies by Self-Assembling Supramolecular Polymers of High Affinity for Protein Binding. Nano Letters 20, 6957-6965 (2020).
42. Huang, L. et al. Novel peptide inhibitors of angiotensin-converting enzyme 2. J Biol Chem 278, 15532-15540 (2003).
43. Behanna, H. A., Donners, J. J. J. M., Gordon, A. C. & Stupp, S. I. Coassembly of Amphiphiles with Opposite Peptide Polarities into Nanofibers. Journal of the American ChemicalSociety 127, 1193-1200 (2005).
44. Amin, M., Sorour, M. K. & Kasry, A. Comparing the Binding Interactions in the Receptor Binding Domains of SARS-CoV-2 and SARS-CoV. The Journal of Physical Chemistry Letters 11, 4897-4900 (2020).
45. Zhang, S. et al. A self-assembly pathway to aligned monodomain gels. Nature Materials 9, 594-601 (2010).
46. Adjei, A. & Gupta, P. Pulmonary delivery of therapeutic peptides and proteins. Journal of Controlled Release 29, 361-373 (1994).
47. Hertel, S. P., Winter, G. & Friess, W. Protein stability in pulmonary drug delivery via nebulization. Advanced Drug Delivery Reviews 93, 79-94 (2015).
48. Berndt, P., Fields, G. B. & Tirrell, M. Synthetic lipidation of peptides and amino acids: monolayer structure and properties. Journal of the American Chemical Society 117, 9515-9522 (1995).
49. Choi, H. S. et al. Rapid translocation of nanoparticles from the lung airspaces to the body. Nature Biotechnology 28, 1300-1303 (2010).
50. Pedersen, S. F. & Ho, Y.-C. SARS-CoV-2: a storm is raging. The Journal of Clinical Investigation 130, 2202-2205 (2020).
51. Antinori, S. et al. Tocilizumab for cytokine storm syndrome in COVID-19 pneumonia: an increased risk for candidemia? Autoimmun Rev 19, 102564-102564 (2020).
52. Zhang, X. et al. Viral and host factors related to the clinical outcome of COVID-19. Nature 583, 437-440 (2020).
53. Lagunas-Rangel, F. A. & Chávez-Valencia, V. High IL-6/IFN-7 ratio could be associated with severe disease in COVID-19 patients. J Med Virol 92, 1789-1790 (2020).
54. Abers, M. S. et al. An immune-based biomarker signature is associated with mortality in COVID-19 patients. JCI Insight 6, e144455 (2021).
55. Ghazavi, A., Ganji, A., Keshavarzian, N., Rabiemajd, S. & Mosayebi, G. Cytokine profile and disease severity in patients with COVID-19. Cytokine 137, 155323 (2021).
56. Kim, M.-H. et al. Type I, II, and III Interferon Signatures Correspond to Coronavirus Disease 2019 Severity. The Journal of Infectious Diseases 224, 777-782 (2021).
57. Zheng, J. et al. COVID-19 treatments and pathogenesis including anosmia in K18-hACE2 mice. Nature 589, 603-607 (2021).
58. Joshi S, Balasubramanian N, Vasam G, Jarajapu YP. Angiotensin converting enzyme versus angiotensin converting enzyrne-2 selectivity of MLN-4760 and DX600 in human and murine bone marrow-derived cells. Eur J Pharmacol. 2016 Mar. 5; 774:25-33.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A supramolecular filament comprising:
an ACE2-binding ligand peptide amphiphile (PA) comprising, in order from N-terminus to C-terminus: i) an aliphatic segment, ii) an intermolecular interaction-regulating peptide segment, iii) a hydrophilic spacer, and iv) an ACE2 inhibitor,
wherein the intermolecular interaction-regulating peptide segment of the ACE2-binding ligand peptide comprises, in order from N-terminus to C-terminus: a hydrogen bond-contributing sequence and one or more charged amino acid residues;
a self-assembling filler peptide amphiphile (PA) comprising, in order from N-terminus to C-terminus: i) an aliphatic segment, and ii) an intermolecular interaction-regulating peptide segment,
wherein the intermolecular interaction-regulating peptide segment of the self-assembling filler PA comprises, in order from N-terminus to C-terminus: a hydrogen bond-contributing sequence and one or more charged amino acid residues that have an opposite charge to the charged amino acid residues of the ACE2-binding ligand PA; and
a soluble ACE2 comprising an S-protein binding site and a carboxypeptidase active site, wherein the soluble ACE2 is bound at the carboxypeptidase active site to the ACE2 inhibitor.

2. The supramolecular filament of claim 1, wherein the aliphatic segment of the ACE2-binding ligand PA or the self-assembling filler PA comprises a dodecyl chain.

3. The supramolecular filament of claim 1, wherein the charged amino acid residues of the self-assembling filler PA comprises:
(i) one or more negatively charged glutamic acid residues; or
(ii) one or more positively charged lysine residues.

4. The supramolecular filament of claim 1, wherein the charged amino acid residues of the ACE2-binding ligand PA comprises one or more positively charged lysine residues and the charged amino acid residues of the self-assembling filler PA comprises one or more negatively charged glutamic acid residues.

5. The supramolecular filament of claim 1, wherein the self-assembling filler PA and the ACE2-binding ligand PA form a filamentous structure comprising electrostatic complexation between the charged amino acid residues of the ACE2-binding ligand PA and the charged amino acid residues of the self-assembling filler PA.

6. The supramolecular filament of claim 1, wherein the hydrogen bond-contributing sequence of the self-assembly filler PA comprises a sequence of VVV (SEQ ID NO: 3).

7. The supramolecular filament of claim 1, wherein the hydrogen bond-contributing sequence of the ACE2-binding ligand PA comprises a sequence of VVV (SEQ ID NO. 3).

8. The supramolecular filament of claim 1, wherein the self-assembling filler PA and the ACE2-binding ligand PA form a filamentous structure comprising a hydrogen bonding network.

9. The supramolecular filament of claim 1, wherein the intermolecular interaction-regulating peptide segment of the ACE2-binding ligand PA comprises a sequence of VVVGKK (SEQ ID NO: 2) and the intermolecular interaction-regulating peptide segment of the self-assembling filler PA comprises a sequence of VVVGEE (SEQ ID NO: 1).

10. The supramolecular filament of claim 1, wherein the hydrophilic spacer comprises a hydrophilic oligoethylene glycol (OEG) chain having between about 4 to 16 OEG units.

11. The supramolecular filament of claim 10, wherein the hydrophilic spacer further comprises a double glycine (GG) sequence.

12. The supramolecular filament of claim 1, wherein the ACE2 inhibitor comprises a chemical structure of:

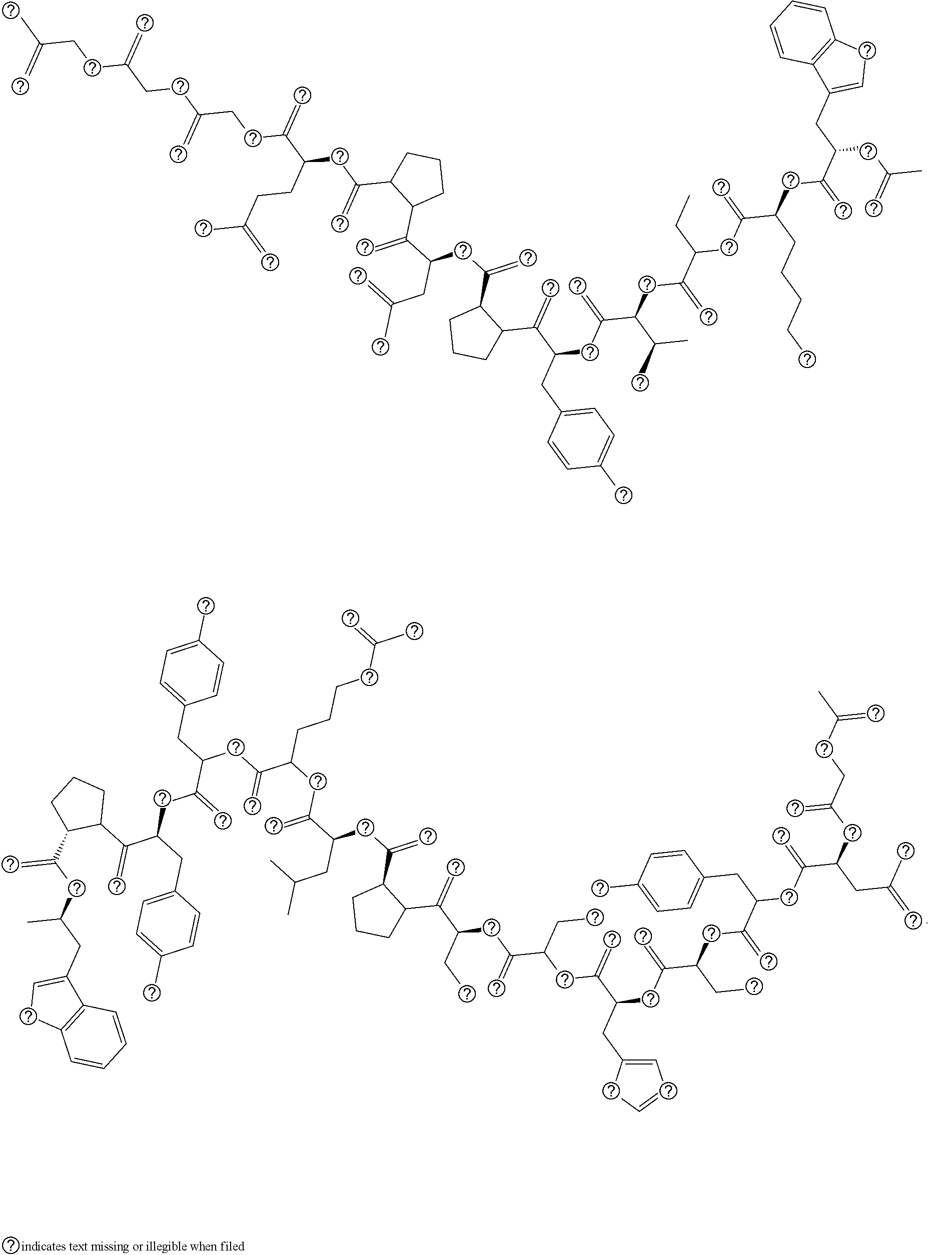

13. The supramolecular filament of claim 1, wherein the ACE2-binding ligand PA comprises amino acid sequence SEQ ID NO:5.

14. The supramolecular filament of claim 1, wherein the ACE2-binding ligand PA comprises amino acid SEQ ID NO: 6.

15. The supramolecular filament of claim 1, wherein the ACE2-binding ligand PA comprises the following structure:

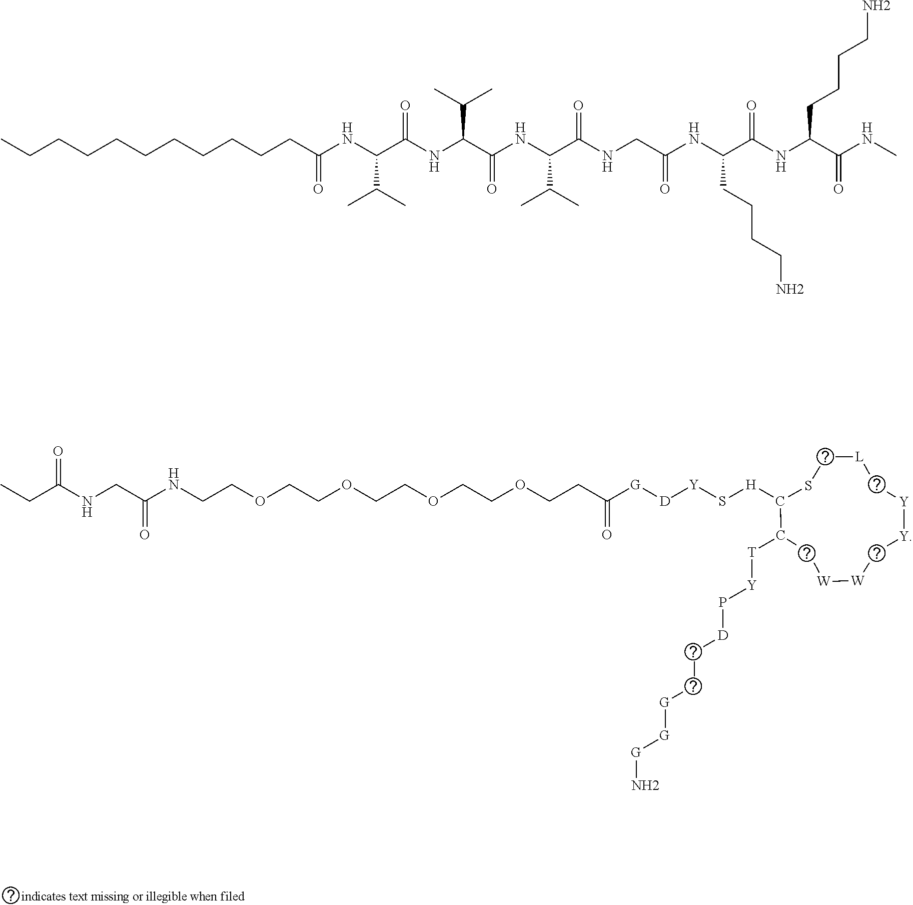

16. The supramolecular filament of claim 1, wherein the ACE2-binding ligand PA has the following chemical structure:

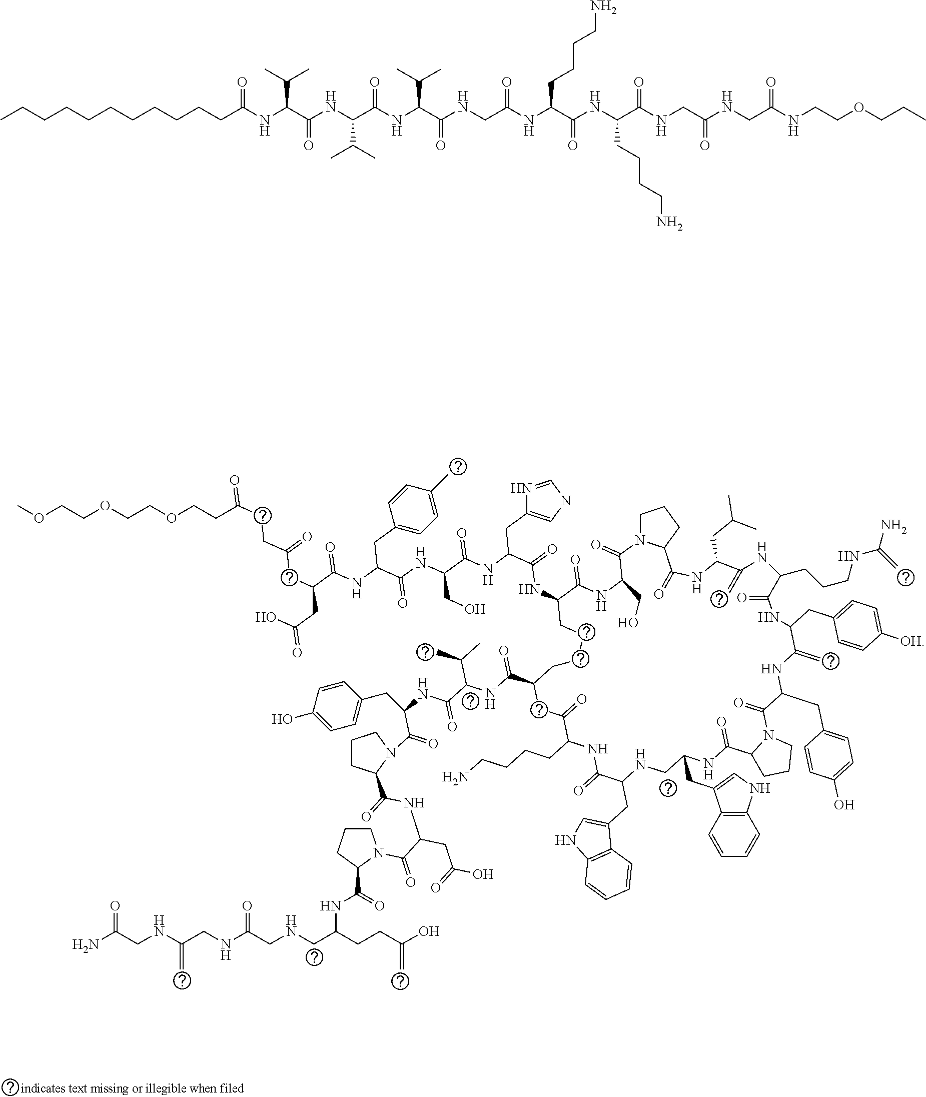

17. The supramolecular filament of claim 1, wherein the intermolecular interaction-regulating peptide segment of the self-assembling filler PA comprises a sequence of VVVGEE (SEQ ID NO: 1).

18. The supramolecular filament of claim 1, wherein the self-assembling filler PA comprises the following chemical structure:

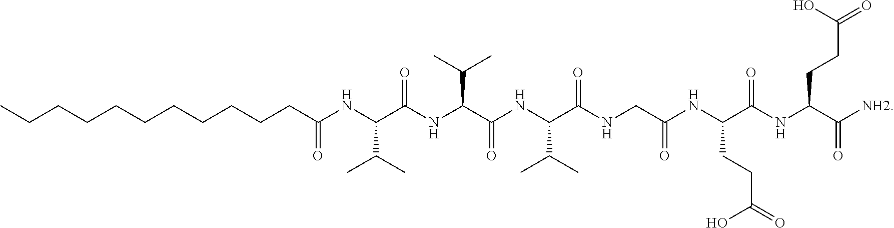 [SMILES: CCCCCCCCCCCC(=O)N[C@H](C(=O)N[C@H](C(=O)N[C@H](C(=O)NCC(=O)N[C@@H](CCC(=O)O)C(=O)N[C@@H](CCC(=O)O)C(N)=O)C(C)C)C(C)C)C(C)C]

19. The supramolecular filament of claim 1, comprising a molar ratio of the self-assembling filler PA to the ACE2-binding ligand PA in the range of about 10:1 to about 50:1.

20. The supramolecular filament of claim 1, wherein the supramolecular filament is biodegradable.

21. A composition comprising the supramolecular filament of claim 1.

22. A respirable aerosol or nasal spray comprising the supramolecular filament of claim 1.

23. A method for treating or preventing a viral infection, the method comprising administering to a subject an amount of the supramolecular filament of claim 1 effective to inhibit viral entry into host cells.

24. The method of claim 23, wherein the method treats or prevents a lung injury or respiratory illness.

25. The method of claim 23, wherein the viral infection comprises a coronavirus infection.

26. The method of claim 25, wherein the coronavirus infection comprises a severe acute respiratory syndrome coronavirus (SARS-CoV) or SARS-CoV-2 infection.

27. The method of claim 26, wherein the coronavirus comprises coronavirus disease of 2019 (COVID-19).

28. The method of claim 23, wherein the supramolecular filament is administered in a respirable aerosol or a nasal spray.
